(19) United States
(12) Patent Application Publication
(10) Pub. No.: US 2006/0014264 A1
(43) Pub. Date: Jan. 19, 2006

(54) Cre/lox system with lox sites having an extended spacer region

(75) Inventors: Brian L. Sauer, Kansas City, MO (US); Vladislav A. Petyuk, Kansas City, MO (US)

Correspondence Address: POLSINELLI SHALTON WELTE SUELTHAUS P.C., 700 W. 47TH STREET, SUITE 1000, KANSAS CITY, MO, 64112-1802, US

(73) Assignee: Stowers Institute for Medical Research

(21) Appl. No.: 11/012,522

(22) Filed: Dec. 15, 2004

Related U.S. Application Data
(60) Provisional application No. 60/587,399, filed on Jul. 13, 2004.

(51) Int. Cl. C12N 9/22

(52) U.S. Cl. 435/199

(57) ABSTRACT

The invention provides a novel Cre/lox system with lox sites having an extended spacer region. In particular, the invention provides Cre mutant polypeptides that can catalyze site-specific recombination at lox sites typically having from one to three additional base pairs in the spacer region. The Cre/lox system can be utilized in a number of genetic manipulations either alone or in combination with other recombinase systems.

### CROSS-REFERENCE TO RELATED APPLICATION

This application claims priority from Provisional Application Ser. No. 60/587,399 filed on Jul. 13, 2004, which is hereby incorporated by reference in its entirety.

### FIELD OF THE INVENTION

The current invention generally relates to a Cre/lox system with lox sites having an extended spacer region. In particular, the invention provides Cre mutant polypeptides that can catalyze site-specific recombination at spatially extended lox sites. The novel Cre/lox system can be utilized in a number of genetic manipulations either alone or in combination with other recombinase systems.

### BACKGROUND OF THE INVENTION

The use of site-specific DNA recombinases has expanded the spectrum of genetic manipulations that can be carried out in both prokaryotic and eukaryotic organisms. While various site-specific DNA recombinases, such as the yeast-derived Flp/frt, are becoming increasingly popular, the Cre/loxP system is currently the most widely used system. Because of its simplicity and versatility, Cre has found widespread use in conditional mutagenesis and gene expression, gene replacement and deletion, and chromosomal engineering experiments.

Cre is a site-specific DNA recombinase derived from the P1 bacteriophage and is a member of the lambda integrase or tyrosine family of site-specific recombinases (1). Members of this family catalyze DNA recombination by a common catalytic mechanism and recognize target recombination sites with similar structural features. In the case of the Cre protein, it recognizes 34 base pair sequences known as loxP sites. The loxP sequence is composed of an asymmetric eight base pair spacer region flanked by 13 base pair inverted repeats. Cre recombines the 34 base pair loxP DNA sequence by binding to the 13 base pair inverted repeats and catalyzing strand cleavage and religation within the spacer region. The staggered DNA cuts made by Cre in the spacer region are separated by 6 base pairs to give an overlap region that acts as a homology sensor to ensure that only recombination sites having the same overlap region recombine. Generally speaking, accepted models of the recombination process by integrase family members can be categorized into five steps (2, 3) following recombinase binding to its target site: (1) DNA synapsis; (2) first strand exchange; (3) Holliday junction conformation change; (4) second strand exchange; and (5) complex release. A catalytic tyrosine residue of the recombinase acts as the catalytic nucleophile to cleave a specific phosphodiester bond on either the top or bottom strand of the target sequence, forming a 3′-O-phosphotyrosine bond to the DNA. Attack of the 3′-O-phosphotyrosine by the free 5′-OH of a second DNA strand then joins the two DNA strands.

One feature of the integrase family of recombinases is that the scissile phosphodiester bonds are located six to eight base pairs apart. This six to eight base pair interval defines the overlap of the crossover region. For many members of the integrase family this interval acts as a homology sensor to ensure that pairs of recombining sites share homology in this region (1). For example, point mutations in the overlap region of the loxP site inhibit recombination with the wild-type loxP site, but recombination of the mutant with itself readily proceeds (4, 5). Generally speaking, the length of the overlap region is characteristic of a particular recombinase (e.g., the overlap region of the target site is six base pairs for Cre and eight base pairs for Flp). Deviation from the naturally occurring spacer length can affect the efficiency of recombination. For example, Flp recombinase activity is abolished by a two base pair insertion in the spacer, but is marginally impacted by either a one base pair insertion or deletion (6). In contrast, lambda integrase does not tolerate even a one base pair deletion or insertion (7, 8).

Not only does specificity for a spacer region having a certain length represent a way to distinguish between recombinases, it also represents a potential means to design new recombinase systems. For example, because of the simplicity and ubiquitous use of the Cre/loxP system in genetic manipulations, a Cre protein that can recognize substrates other than the loxP site would be highly beneficial as a research tool either alone or in combination with the current Cre/loxP system. This is particularly true considering the wild-type Cre protein's tolerance for some insert mutations results in dramatically lower recombination rates in both E. coli and yeast (9, 10, 11). Accordingly, a Cre polypeptide that could catalyze a high rate of recombination at a lox site having an extended spacer region would provide a novel Cre/lox system with a higher degree of specificity relative to the current Cre/loxP system. While several attempts to alter the site specificity of Cre have had some success, each of these attempts focused on altering the DNA-binding specificity of Cre to the 13 base pair inverted repeat elements of the lox site (21-24). Cre mutant polypeptides that can efficiently catalyze site-specific recombination at a lox site having an extended spacer region have not been previously characterized.

### SUMMARY OF THE INVENTION

Among the several aspects of the current invention, therefore, is the provision of a Cre/lox system having a lox site with additional nucleotide base pairs within the spacer region. The invention provides novel Cre mutant polypeptides that can catalyze site specific recombination or excision at a mutant lox site having additional nucleotide base pairs in the spacer region. In contrast, wild-type Cre polypeptides catalyze site specific recombination at a mutant lox site having additional nucleotide base pairs in the spacer region at a lower efficiency compared to the Cre mutant polypeptides of the current invention. Advantageously, because of this difference in substrate specificity, the novel Cre/lox system of the present invention provides an additional tool that may be utilized either alone or in combination with other Cre/lox systems for conditional mutagenesis and gene expression, gene replacement and deletion, and chromosome engineering. Moreover, because the Cre mutant polypeptides of the invention recognize a substrate having more nucleotide base pairs compared to wild-type Cre polypeptides, the Cre/lox system of the present invention has a higher degree of specificity relative to the Cre/loxP system.

Briefly, therefore, one aspect of the present invention encompasses a purified Cre mutant polypeptide that can catalyze site specific recombination at a lox site having additional nucleotide base pairs in the spacer region. In one alternative of this embodiment, the mutant polypeptide has an amino acid sequence such that it specifically binds to an antibody that binds specifically to a Cre wild-type polypeptide having SEQ ID NO. 1. In yet another alternative of this embodiment, the Cre mutant polypeptide has an amino acid sequence that comprises SEQ ID NO. 1 with 5 additional amino acids inserted consecutively within the J-K loop. In a further alternative of this embodiment, the Cre mutant polypeptide has an amino acid sequence that comprises SEQ ID NO. 1 with 5 additional amino acids inserted consecutively within the N-terminus of helix A. In still another alternative of this embodiment, the Cre mutant polypeptide has an amino acid sequence such that it specifically binds to an antibody that binds specifically to a polypeptide having a sequence selected from the group consisting of SEQ ID NOs. 6-17.

Yet another aspect of the invention provides isolated nucleotide sequences that encode Cre mutant polypeptides that can catalyze site specific recombination at a lox site having additional nucleotide base pairs in the spacer region. In one alternative of this embodiment, the isolated nucleotide sequence comprises a sequence that encodes a polypeptide having an amino acid sequence selected from the group consisting of SEQ ID NOs. 2-5, or of a fragment of any of SEQ ID NOs. 2-5 that is at least 15 amino acid residues in length. In another alternative of this embodiment, the isolated nucleotide sequence comprises a sequence that hybridizes under stringent conditions to a hybridization probe the nucleotide sequence of which encodes a polypeptide having an amino acid sequence selected from the group consisting of SEQ ID NOs. 2-5.

A further aspect of the invention provides purified antibodies that are specific for a Cre mutant polypeptide of the invention. In one embodiment, the purified antibody binds specifically to a polypeptide having an amino acid sequence selected from the group consisting of SEQ ID NOs. 2-5. The purified antibodies may be either monoclonal or polyclonal antibodies and may be used to purify Cre mutant polypeptides of the present invention.

An additional aspect of the invention encompasses an isolated mutant lox nucleotide sequence having additional nucleotide base pairs in the spacer region. In one embodiment, the isolated lox nucleotide sequence comprises a sequence at least 50% identical to SEQ ID. Nos. 18 or 139 with from one to three additional nucleotides in the spacer region. In one alternative of this embodiment, the additional nucleotides in the spacer region are selected from the group consisting of adenosine 5′-monophosphate and thymidine 5′-monophosphate. In another alternative of this embodiment, the additional nucleotides in the spacer region are selected from the group consisting of guanosine 5′-monophosphate and cytidine 5′-monophosphate. In still another alternative of this embodiment, the additional nucleotides in the spacer region are selected from the group consisting of adenosine 5′-monophosphate, thymidine 5′-monophosphate guanosine, 5′-monophosphate and cytidine 5′-monophosphate.

Yet another aspect of the invention encompasses a Cre/lox system. The system typically comprises a purified mutant Cre polypeptide that can catalyze site specific recombination at a lox site having additional nucleotides in the spacer region; and an isolated lox nucleotide sequence with additional nucleotides in the spacer region.

A further aspect of the invention provides a method for producing site-specific recombination of nucleotide sequence having a target DNA segment. The method involves introducing a first lox site and a second lox site into the nucleotide sequence such that the lox sites flank the target DNA segment, wherein each of the lox sites have additional nucleotides in the spacer region. The lox sites are then contacted with a Cre mutant polypeptide that can catalyze site specific recombination at a lox site having additional nucleotides in the spacer region. When the Cre mutant polypeptide is contacted with the lox sites, site specific recombination of the nucleotide sequence occurs.

An additional aspect of the invention encompasses a kit for producing site-specific recombination of nucleotide sequence. Typically, the kit will comprise a purified mutant Cre polypeptide that can catalyze site specific recombination at a lox site having additional nucleotides in the spacer region; an isolated lox nucleotide sequence with additional nucleotides in the spacer region; and instructions for producing site specific recombination.

A further aspect of the invention encompasses cells and nucleic acid sequences having the Cre mutant polypeptides and mutant lox sites of the invention.

Other objects and features of the invention will be in part apparent and in part pointed out hereinafter.

### BRIEF DESCRIPTION OF THE FIGURES

FIG. 1 depicts the isolation of Cre mutants proficient in lox⁺³ recombination. (A) Activation of the neo gene by excisive recombination. The Ap^{R} reporter plasmid carries two directly repeated lox⁺³ sites flanking a rrn T1T2 transcription terminator (Term) interposed between the lac promoter and neo. Cre-mediated excision at the lox sites allows neo expression to give kanamycin resistance. (B) Enrichment of active Cre mutants after successive rounds of selection. The percent recombination (ratio of Kn^{R} to total number of transformants) of the Cm^{R} cre plasmid into the Ap^{R} lox⁺³ reporter strain is shown for the original insertion library (lib) and after successive rounds of selection. Pools from which individual Cre-expressing plasmids were sequenced are labeled with asterisks. For comparison the same assay is shown with the wt cre plasmid and E. coli DH5α carrying the loxP reporter pBS848 (25). (C) Western blotting. Cre expression after 1 hr of arabinose induction is shown for the indicated mutants, the wt Cre vector and for vector with no cre gene (−). Coding region mutants are marked with an asterisk. (D) Location of mutants chosen for detailed characterization. The secondary structure of Cre (grey cylinder=α-helix, black arrow=strand) is from the published crystal structure (26).

FIG. 2 depicts recombination in vitro. Cre mutants are designated by the amino acid position of insertion labeled, with “18” being the double mutant 18::CGRNA+P15L. (A) Intramolecular excisive recombination with a lox+3 substrate. Following recombination DNA was linearized by restriction digestion to facilitate analysis. Bands corresponding to non-recombined substrate (non-rec), recombined products (rec) and Holliday junctions (HJ) are indicated. Size markers are shown to the right. A faint 7 kb band from incomplete restriction is present in all lanes. (B) Comparison of mutant Cre recombination at wt and extended spacer mutant lox sites. Intramolecular recombination was assayed as above using appropriate lox² substrates: loxP (P, white), lox⁺¹ (1, striped), lox⁺² (2, grey) and lox⁺³ (3, black). Solid bars represent complete recombination products and dashed bars represent Holiday junction products.

FIG. 3 depicts a substrate cleavage assay. Cre-mediated cleavage was assayed using 2 nM of the ³²P-labelled lox⁺³ oligonucleotide substrate and 30 nM Cre, followed by SDS gel electrophoresis. Cre mutants are designated above the gel as in FIG. 2. Diagrams indicate bands corresponding to uncleaved DNA (free) and DNA covalently linked to the catalytic tyrosine of Cre (cov). The cleavage efficiency relative to wt Cre is indicated below the corresponding lane for each mutant. The position of the ³²P label is denoted by an asterisk.

FIG. 4 depict the formation of a synaptic complex. Intramolecular synaptic complex formation was with 10 nM of the indicated Cre mutant and a 544 bp ³²P-labelled DNA fragment (0.05 nM) having two lox⁺³ sites in inverted orientation. Diagrams representing unbound (free), unsynapsed DNA fragment bound with four Cre monomers (c4) and the synaptic complex are shown adjacent to the corresponding bands. Cre mutants are designated as in FIG. 2 except that Y324F derivatives were used to prevent catalysis.

FIG. 5 depicts a schematic of a ribbon model showing the interaction of Cre at a loxP site. Briefly, two Cre subunits are shown in blue and yellow. Amino acid residues 20 and 24 are shown in green and amino acid residues 280 and 286 are shown in magenta.

FIG. 6 depicts a schematic showing sequence alignment of Cre recombinase homologs having SEQ ID Nos. 1, 6-17, and 140. Sequence numbering is from top to bottom, such that Cre is SEQ ID. No. 1 and XerD is SEQ ID. No. 140.

FIG. 7 is a schematic illustrating use of the Cre/lox system in transgenic mice. Mice with Cre protein expression in a specific cell type are bred to mice that contain a target gene surrounded by loxP sites. When the mice are bred, cells that have expressed Cre will lose the target gene and one lox site.

### DETAILED DESCRIPTION OF THE PREFERRED EMBODIMENTS

The present invention provides novel Cre mutant polypeptides and mutant lox sites that can be utilized in a novel Cre/lox system. In its most basic use, the wild-type Cre/loxP recombination system of bacteriophage P1 may be employed as a method to selectively delete a specific portion of target DNA. The loxP sites work in pairs and they flank a segment of a target DNA molecule. The following schematic depicts a nucleotide sequence having a target DNA flanked by two loxP sites in the same orientation: 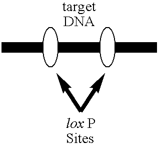
When the Cre polypeptide is contacted with the loxP sites, it binds to the sites and exchanges DNA strands between the sites and in so doing excises the target DNA as a circular molecule. After the Cre polypeptide has excised the target DNA, one lox site is left behind and the two flanking fragments of DNA are spliced together. The following schematic depicts the DNA molecule shown above after the molecule has been contacted with a Cre polypeptide.

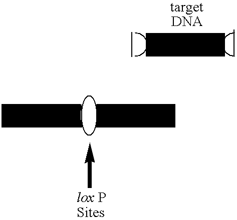

The Cre mutant polypeptides of the present invention perform site specific recombination in a manner identical to wild-type Cre as depicted above, but the Cre mutant polypeptides recognize different substrate sites. In particular, the Cre mutant polypeptides can catalyze site specific recombination or excision at a mutant lox site having additional base pairs in the spacer region at a higher efficiency compared to wild-type Cre. Because of this difference in substrate specificity, advantageously, the novel Cre/lox system of the present invention provides an additional tool that may be utilized either alone or in combination with other Cre/lox systems for conditional mutagenesis and gene expression, gene replacement and deletion, and chromosome engineering.

Cre Mutant Polypeptides

The Cre mutant polypeptides of the present invention, as exemplified in the examples, can typically catalyze site specific recombination or excision at a spatially extended lox site at a higher efficiency compared to a wild-type Cre polypeptide. In one embodiment, the Cre mutant polypeptide can catalyze site specific recombination or excision at a lox site having from one to approximately ten additional base pairs in its spacer region. In a more preferred embodiment, the Cre mutant polypeptide can catalyze site specific recombination or excision at a lox site having from one to approximately five additional base pairs in its spacer region. In an even more preferred embodiment, the Cre mutant polypeptide can catalyze site specific recombination or excision at a lox site having from one to approximately three additional base pairs in its spacer region. In one alternative of this embodiment, the lox site will have one additional base pair in its spacer region. In a further alternative of this embodiment, the lox site will have two additional base pairs in its spacer region. In yet another alternative of this embodiment, the lox site will have three additional base pairs in its spacer region. Suitable lox sites having extended spacer regions are detailed below. The Cre mutant polypeptides can also typically catalyze site specific recombination or excision at a wild-type lox site. Generally speaking, the Cre mutant polypeptides share substantial sequence homology with the wild-type Cre polypeptide isolated from bacteriophage P1 having SEQ ID NO. 1.

In one aspect of the invention, the mutant polypeptide has an amino acid sequence such that it specifically binds to an antibody that binds specifically to a Cre wild-type polypeptide having SEQ ID NO. 1. Typically, mutant polypeptides in this embodiment will have an amino acid sequence that is at least 50% identical to SEQ ID NO.1, and more typically, the mutant polypeptide will have an amino acid sequence that is at least 75% identical to SEQ ID NO.1. Exemplary mutant polypeptides, however, will have an amino acid sequence that is at least 90%, more preferably 95%, and even more preferably, 99% identical to SEQ ID NO. 1. In a further alternative of this embodiment, the mutant polypeptide will have an amino acid sequence that comprises SEQ. ID. NO.1 with 1 to 50 conservative amino acid substitutions. In an exemplary alternative of this embodiment, the mutant polypeptide will have an amino acid sequence that comprises SEQ ID NO. 1 with 1 to 15, and more typically, from 1 to 10 conservative amino acid substitutions. In each of these embodiments, typically the mutant polypeptide can catalyze site specific recombination or excision at a lox site having from one to three additional nucleotides in the spacer region at a higher efficiency compared to wild-type Cre.

Yet another aspect of the invention encompasses a Cre mutant polypeptide that comprises SEQ ID NO. 1 with from one to about five additional amino acids inserted consecutively within the N-terminus of helix A. By way of example, in one embodiment the additional amino acids may be inserted after any of residues 1 to about 30 of SEQ ID NO. 1. By way of further example, the additional amino acids may be inserted after any of residues 1 to 5, 5 to 10, 10 to 15, 15 to 20, 20 to 25, or 25 to 30 of SEQ ID NO. 1. More typically, however, the additional amino acids are inserted from about residue 17 to about 25 of SEQ ID NO. 1. In an exemplary embodiment, five additional amino acids are inserted after either residue 18 or residue 24 of SEQ ID NO. 1. In one preferred embodiment, the five additional residues are inserted after residue 18 of SEQ ID NO. 1. An example of a Cre mutant polypeptide with five additional amino acid residues after residue 18 is shown in the examples and has an amino acid sequence comprising SEQ ID NO. 2. In yet another preferred embodiment, the five additional residues are inserted after residue 24 of SEQ ID NO. 1. An example of a Cre mutant polypeptide with five additional amino acid residues after residue 24 is shown in the examples and has an amino acid sequence comprising SEQ ID NO. 3. In still other alternatives of this embodiment, the Cre mutant polypeptide will have an amino acid sequence that is at least 50% identical to SEQ ID NO. 2 or 3, and more typically, the mutant polypeptide will have an amino acid sequence that is at least 75% identical to SEQ ID NO. 2 or 3. Exemplary mutant polypeptides in this embodiment, however, will have an amino acid sequence that is at least 90%, more preferably 95%, and even more preferably, 99% identical to SEQ ID NO. 2 or 3. In a further alternative of this embodiment, the mutant polypeptide will have an amino acid sequence that comprises SEQ. ID. NO. 2 or 3 with 1 to 50 conservative amino acid substitutions. In an exemplary alternative of this embodiment, the mutant polypeptide will have an amino acid sequence that comprises SEQ ID NO. 2 or 3 with 1 to 15, and more typically, from 1 to 10 conservative amino acid substitutions. In each of these embodiments, typically the mutant polypeptide can catalyze site specific recombination or excision at a lox site having from one to three additional nucleotides in the spacer region at a higher efficiency compared to wild-type Cre.

A further aspect of the invention embraces Cre mutant polypeptides that comprise SEQ ID NO. 1 with from one to five additional amino acids inserted consecutively in the loop between the J and K helices. By way of example, in one embodiment the additional amino acids may be inserted after any of residues 270 to about 290 of SEQ ID NO. 1. By way of further example, the additional amino acids may be inserted after any of residues 270 to 275, 275-280, 280-285, or 285-290 of SEQ ID NO. 1. More typically, however, the additional amino acids are inserted from about residue 279 to about 287 of SEQ ID NO. 1. In an exemplary embodiment, five additional amino acids are inserted after either residue 280 or residue 287 of SEQ ID NO. 1. In one preferred embodiment, the five additional residues are inserted after residue 280 of SEQ ID NO. 1. An example of a Cre mutant polypeptide with five additional amino acid residues after residue 280 is shown in the examples and has an amino acid sequence comprising SEQ ID NO. 4. In yet another preferred embodiment, the five additional residues are inserted after residue 286 of SEQ ID NO. 1. An example of a Cre mutant polypeptide with five additional amino acid residues after residue 286 is shown in the examples and has an amino acid sequence comprising SEQ ID NO. 5. Generally speaking, mutant polypeptides in this embodiment will have an amino acid sequence that is at least 50% identical to SEQ ID NO. 4 or 5, and more typically, the mutant polypeptide will have an amino acid sequence that is at least 75% identical to SEQ ID NO. 4 or 5. Exemplary mutant polypeptides, however, will have an amino acid sequence that is at least 90%, more preferably 95%, and even more preferably, 99% identical to SEQ ID NO. 4 or 5. In a further alternative of this embodiment, the mutant polypeptide will have an amino acid sequence that comprises SEQ. ID. NO. 4 or 5 with 1 to 50 conservative amino acid substitutions. In an exemplary alternative of this embodiment, the mutant polypeptide will have an amino acid sequence that comprises SEQ ID NO. 4 or 5 with 1 to 15, and more typically, from 1 to 10 conservative amino acid substitutions. In each of these embodiments, typically the mutant polypeptide can catalyze site specific recombination or excision at a lox site having from one to three additional nucleotides in the spacer region at a higher efficiency compared to wild-type Cre.

Because of the somewhat ubiquitous nature of the Cre polypeptides, it will be appreciated by those skilled in the art that additional suitable Cre polypeptides exist in species other than the ones specifically detailed herein. It will also be appreciated that additional polypeptides may be present in a species in addition to the polypeptides detailed herein. The invention contemplates the use of all suitable Cre mutant polypeptides having the structure and function as described herein.

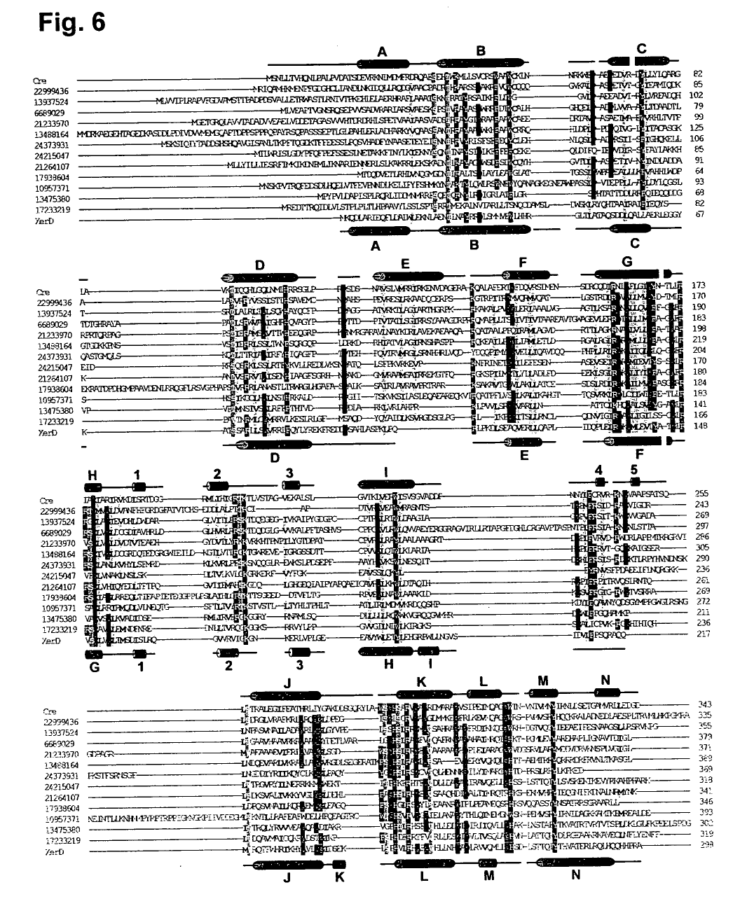

In certain aspects, accordingly, a polypeptide that is a homolog, ortholog, or degenerative variant of a Cre mutant polypeptide is also suitable for use in the present invention. Typically, the subject polypeptides include fragments that share substantial sequence similarity, binding specificity and function with any of the polypeptides detailed above, including wild-type Cre polypeptide isolated from bacteriophage P1 having SEQ ID NO. 1, or such as those polypeptides having SEQ ID Nos. 2, 3, 4 or 5. For example, as detailed in FIG. 6, the polypeptide of each of SEQ ID Nos. 6 through 17 are homologs to Cre polypeptide from bacteriophage P1 having SEQ ID NO. 1. In one alternative of this embodiment, the Cre mutant polypeptide has an amino acid sequence such that it specifically binds to an antibody that binds specifically to any of SEQ ID Nos. 6 through 17. In each of these embodiments, typically the mutant polypeptide can catalyze site specific recombination or excision at a lox site having from one to three additional nucleotides in the spacer region at a higher efficiency compared to wild-type.

A number of methods may be employed to determine whether a particular homolog or degenerative variant possesses substantially similar biological activity relative to a Cre mutant polypeptide of the invention. In particular, the subject polypeptide, if suitable for use in the invention, will be able to catalyze site specific recombination or excision at a lox site having from about one to about three additional nucleotides in the spacer region at a higher efficiency than wild-type Cre. In order to determine whether a particular polypeptide can function in this manner, either the in vitro or in vivo recombination assays detailed in the examples may be followed.

In determining whether a polypeptide is substantially homologous or shares a certain percentage of sequence identity with a Cre mutant polypeptide of the invention, sequence similarity may be determined by conventional algorithms, which typically allow introduction of a small number of gaps in order to achieve the best fit. In particular, “percent homology” of two polypeptides or two nucleic acid sequences is determined using the algorithm of Karlin and Altschul (Proc. Natl. Acad. Sci. USA 87:2264-2268, 1993). Such an algorithm is incorporated into the NBLAST and XBLAST programs of Altschul et al. (J. Mol. Biol. 215:403-410, 1990). BLAST nucleotide searches may be performed with the NBLAST program to obtain nucleotide sequences homologous to a nucleic acid molecule of the invention. Equally, BLAST protein searches may be performed with the XBLAST program to obtain amino acid sequences that are homologous to a polypeptide of the invention. To obtain gapped alignments for comparison purposes, Gapped BLAST is utilized as described in Altschul et al. (Nucleic Acids Res. 25:3389-3402, 1997). When utilizing BLAST and Gapped BLAST programs, the default parameters of the respective programs (e.g., XBLAST and NBLAST) are employed. See http://www.ncbi.nlm.nih.gov for more details.

Cre mutant polypeptides suitable for use in the invention are typically isolated or pure. An “isolated” polypeptide is unaccompanied by at least some of the material with which it is associated in its natural state, preferably constituting at least about 0.5%, and more preferably, at least about 5% by weight of the total polypeptide in a given sample. A pure polypeptide constitutes at least about 90%, preferably, 95% and even more preferably, at least about 99% by weight of the total polypeptide in a given sample.

The Cre mutant polypeptide may be synthesized, produced by recombinant technology, or purified from cells. In one embodiment, the Cre mutant polypeptide of the present invention may be obtained by direct synthesis. In addition to direct synthesis, the subject polypeptides can also be expressed in cell and cell-free systems (e.g. Jermutus L, et al., Curr Opin Biotechnol. October 1998; 9(5):534-48) from encoding polynucleotides, such as described below or naturally-encoding polynucleotides isolated with degenerate oligonucleotide primers and probes generated from the subject polypeptide sequences (“GCG” software, Genetics Computer Group, Inc, Madison Wis.) or polynucleotides optimized for selected expression systems made by back-translating the subject polypeptides according to computer algorithms (e.g. Holler et al. (1993) Gene 136, 323-328; Martin et al. (1995) Gene 154, 150-166). In other embodiments, any of the molecular and biochemical methods known in the art are available for biochemical synthesis, molecular expression and purification of the Cre mutant polypeptide, see e.g. Molecular Cloning, A Laboratory Manual (Sambrook, et al. Cold Spring Harbor Laboratory), Current Protocols in Molecular Biology (Eds. Ausubel, et al., Greene Publ. Assoc., Wiley-Interscience, New York).

Cre Nucleotide Sequences

The present invention also encompasses the use of isolated nucleotide sequences that encode suitable Cre mutant polypeptides. For example, the subject nucleotide sequences may be utilized as a means to produce a Cre mutant polypeptide having the structure and biological activity as detailed above.

The nucleotide sequence may be any of a number of such nucleotide sequences that encode a suitable Cre mutant polypeptide, having the structure and function as described herein. In one embodiment, the isolated nucleotide is a sequence that encodes a polypeptide having an amino acid sequence selected from the group consisting of SEQ ID NO. 2, 3, 4, or 5, or of a fragment of any of SEQ ID NO. 2, 3, 4, or 5 that is at least 15 amino acid residues in length.

In still another embodiment, the isolated nucleotide sequence will encode a polypeptide that has an amino acid sequence that is at least 50% identical to the amino acid sequence of any of SEQ ID NO. 2, 3, 4, or 5. More typically, however, the isolated nucleotide sequence will encode a polypeptide that has an amino acid sequence that is at least 75% identical to the amino acid sequence of any of SEQ ID NO. 2, 3, 4, or 5 and even more typically, 90% identical to the amino acid sequence of any of SEQ ID NO. 2, 3, 4, or 5. In a particularly preferred embodiment, the nucleotide sequence will encode a polypeptide that has an amino acid sequence that is at least 95%, and even more preferably, 99% identical to the amino acid sequence of any of SEQ ID NO. 2, 3, 4, or 5. In each of these embodiments, the isolated nucleotide sequence will preferably encode a polypeptide that will be able to catalyze site specific recombination or excision at a lox site having from about one to three additional nucleotides in the spacer region at a higher efficiency compared to wild-type Cre.

The invention also encompasses the use of nucleotide sequences other than a sequence that encodes a polypeptide having an amino acid sequence selected from the group consisting of SEQ ID NO. 2, 3, 4, or 5. Typically, these nucleotide sequences will hybridize under stringent hybridization conditions (as defined herein) to all or a portion of the nucleotide sequences described above or their complement. The hybridizing portion of the hybridizing nucleic acids is usually at least 15 (e.g., 20, 25, 30, or 50) nucleotides in length. The hybridizing portion of the hybridizing nucleic acid is at least 80%, preferably, at least 90%, and is more preferably, at least 95% identical to the sequence of a portion or all of a nucleic acid sequence encoding a Cre mutant polypeptide suitable for use in the present invention, or its complement.

Hybridization of the oligionucleotide probe to a nucleic acid sample is typically performed under stringent conditions. Nucleic acid duplex or hybrid stability is expressed as the melting temperature or Tm, which is the temperature at which a probe dissociates from a target DNA. This melting temperature is used to define the required stringency conditions. If sequences are to be identified that are related and substantially identical to the probe, rather than identical, then it is useful to first establish the lowest temperature at which only homologous hybridization occurs with a particular concentration of salt (e.g., SSC or SSPE). Then, assuming at 1% mismatching results in a 1° C. decrease in the Tm, the temperature of the final wash in the hybridization reaction is reduced accordingly. For example, if sequences have greater than 95% identity with the probe is sought, the final temperature is approximately decreased by 5° C. In practice, the change in Tm can be between 0.5 and 1.5° C. per 1% mismatch. Stringent conditions involve hybridizing at 68° C. in 5×SSC/5× Denhardt's solution/1.0% SDS, and washing in 0.2×SSC/0.1% SDS at room temperature. Moderately stringent conditions include washing in 3×SSC at 42° C. The parameters of salt concentration and temperature can be varied to achieve the optimal level of identity between the probe and the subject nucleotide sequence. Additional guidance regarding such conditions is readily available in the art, for example, by Sambrook et al., 1989, Molecular Cloning, A Laboratory Manual, Cold Spring Harbor Press, N.Y.; and Ausubel et al., (eds.), 1995, Current Protocols in Molecular Biology, (John Wiley & Sons, N.Y.) at Unit 2.10.

The various nucleic acid sequences mentioned above can be obtained using a variety of different techniques known in the art. The nucleotide sequences, as well as homologous sequences encoding a suitable Cre mutant polypeptide, can be isolated using standard techniques, or can be purchased or obtained from a depository. Once the nucleotide sequence is obtained, it can be amplified for use in a variety of applications, as further described below.

The invention also encompasses production of nucleotide sequences that encode suitable Cre mutant polypeptide homologs, derivatives, or fragments thereof, that may be made by any method known in the art, including by synthetic chemistry. After production, the synthetic sequence may be inserted into any of the many available expression vectors and cell systems using reagents well known in the art. Moreover, synthetic chemistry may be used to introduce additional mutations into a nucleotide sequence encoding a suitable Cre mutant polypeptide.

The nucleotide sequences of the present invention can be engineered using methods generally known in the art in order to alter Cre mutant polypeptides-encoding sequences for a variety of purposes including, but not limited to, modification of the cloning, processing, and/or expression of the gene product. DNA shuffling by random fragmentation and PCR reassembly of gene fragments and synthetic oligonucleotides may be used to engineer the nucleotide sequences. For example, oligonucleotide-mediated site-directed mutagenesis may be used to introduce mutations that create new restriction sites, alter glycosylation patterns, change codon preference, produce splice variants, and so forth.

Lox Site Nucleotide Sequences

The invention also encompasses a number of mutant lox sites that have a spatially extended spacer region. The mutant lox sites typically function as substrate sites for the Cre mutant polypeptide of the invention. Generally speaking, a wild-type lox site will typically consist of two oppositely oriented perfect repeats that are separated by a spacer region. For example, the loxP site consists of two 13 base pair inverted repeats separated by an 8 base pair spacer region. The nucleotide sequence of the wild-type loxP site is as follows:

### Wild Type loxP Site (34 bp)

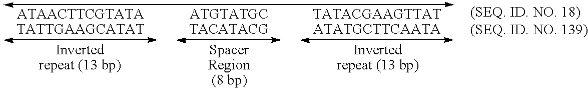

Mutant lox sites suitable for use in the present invention typically have two inverted repeat regions that are identical or substantially identical to a wild-type lox site, but have additional nucleotide base pairs with varying sequences, depending upon the embodiment, in the spacer region. An example of a suitable mutant lox site is represented by the following formula (I):
R₁—X—R₁ (I)
wherein:
R₁ is an inverted repeat region; and
X is a spacer region with at least one additional nucleotide base pair compared to a corresponding wild-type spacer region.

Suitable mutant lox sites represented by formula (I) include nucleotide sequences at which the Cre mutant polypeptides of the invention can catalyze site-specific recombination. By way of non-limiting example, the lox site may be a mutant of any of loxP, loxB, loxL, or loxR. Generally speaking, the inverted repeat region of a mutant lox site having formula (I) is the same nucleotide length and sequence as a corresponding wild-type lox site. The spacer region of a mutant lox site having formula (I) will be at least one additional nucleotide base pair longer than a corresponding wild-type lox site and may include a number of sequence substitutions depending upon the particular embodiment, as further described below. In a more typical embodiment, the spacer region of the mutant lox site will have from one to about ten, even more typically, from one to about five and most typically, from one to three additional nucleotide base pairs compared to a corresponding wild-type lox site.

In one exemplary embodiment for mutant lox sites having formula (I), the spacer region contains the same nucleotide sequence as a corresponding wild-type lox site, but has three additional nucleotide base pairs in the spacer region. The choice of placement of the three additional nucleotide base pairs within the spacer region is generally not a critical aspect of the invention. Typically, the three additional nucleotide base pairs can be inserted before or after any single nucleotide within the wild-type spacer region. In one embodiment, the three additional nucleotide base pairs are inserted within the wild-type spacer region consecutively so that the nucleotides appear within the spacer region one right after another. In an alternative embodiment, the three additional nucleotide base pairs are inserted within the spacer region so that two of the nucleotides are inserted consecutively, i.e., one right after the other, and the other nucleotide base pair is inserted before or after any single nucleotide in the wild-type spacer region, but not next to the two other inserted nucleotide base paris. In a further alternative embodiment, the three additional nucleotide base pairs are singly inserted within the wild-type spacer region so that none of the inserted nucleotides are next to one and another. The three additional nucleotide base pairs are generally selected so as to include nitrogenous bases from either the purine or the pyrimidine chemical classes. But this choice is also typically not a critical feature of the invention to the extent that the base pairs are complementary. For example, the three additional nucleotides may be all purines or all pyrimidines. The three additional nucleotides may be two purines and one pyrimidine. Alternatively, the three nucleotides may include one purine and two pyrimidines. Suitable purines include adenine, guanine, hypoxanthine and xanthine. In exemplary embodiments, the purine will be either adenine or guanine. Suitable pyrimidines include cytosine, uracil or thymine.

In yet another exemplary embodiment for mutant lox sites having formula (I), the spacer region contains the same nucleotide sequence as a corresponding wild-type lox site, but has two additional nucleotide base pairs in the spacer region. The choice of placement of the two additional nucleotide base pairs within the spacer region is generally not a critical aspect of the invention. Typically, the two additional nucleotide base pairs can be inserted before or after any single nucleotide within the wild-type spacer region. In one embodiment, the two additional nucleotide base pairs are inserted within the wild-type spacer region consecutively so that the nucleotides appear within the spacer region one right after another. In a further alternative embodiment, the two additional nucleotide base pairs are singly inserted within the wild-type spacer region so that the inserted nucleotides are not next to one and another. The two additional nucleotide base pairs are generally selected so as to include nitrogenous bases from either the purine or the pyrimidine chemical classes, which may include any of the purines or pyrimidines discussed above.

In a further exemplary embodiment for mutant lox sites having formula (I), the spacer region contains the same nucleotide sequence as a corresponding wild-type lox site, but has one additional nucleotide base pair in the spacer region. The choice of placement of the additional nucleotide base pair within the spacer region is generally not a critical aspect of the invention and it may typically be inserted before or after any single nucleotide within the wild-type spacer region. The additional nucleotide base pair is generally selected so as to include nitrogenous bases from either the purine or the pyrimidine chemical classes, which may include any of the purines or pyrimidines discussed above.

In a preferred embodiment, the mutant lox site is a loxP site represented by the following formula (II) 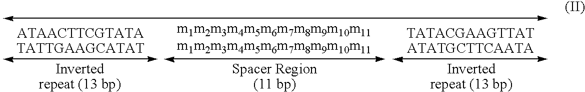
wherein:
m₁, m₂, m₃, m₄, m₅, m₆, m₇, m₈, m₉, m₁₀, and m₁₁ together comprise the spacer region and are independently a complementary nucleotide base pair wherein the nitrogenous base is a purine or a pyrimidine.

In each embodiment for mutant loxP sites having formula (II) described herein, the inverted repeat region comprises the two 13 base pair inverted repeats of the wild-type loxP separated by an eleven base pair spacer region.

Alternatively, the spacer region may include a number of different nucleotide base pair sequences to the extent that the sequence selected can serve as a substrate for the Cre mutant polypeptides of the invention. In one alternative of this embodiment, approximately 75% of m₁, m₂, m₃, m₄, m₅, m₆, m₇, m₈, m₉, m₁₀, and m₁₁ comprise an adenine-thymine complementary nucleotide base pair. In another alternative of this embodiment, approximately 75% to 80% of m₁, m₂, m₃, m₄, m₅, m₆, m₇, m₈, m₉, m₁₀, and m₁₁ comprise an adenine-thymine complementary nucleotide base pair. In still another alternative embodiment, approximately 80% to 85% of m₁, m₂, m₃, m₄, m₅, m₆, m₇, m₈, m₉, m₁₀, and m₁₁ comprise an adenine-thymine complementary nucleotide base pair. In yet another of alternative embodiment, approximately 85% to 90% of m₁, m₂, m₃, m₄, m₅, m₆, m₇, m₈, m₉, m₁₀, and m₁₁ comprise an adenine-thymine complementary nucleotide base pair. In a further alternative of this embodiment, approximately 90% to 95% of m₁, m₂, m₃, m₄, m₅, m₆, m₇, m₈, m₉, m₁₀, and m₁₁ comprise an adenine-thymine complementary nucleotide base pair. In yet another alternative embodiment, 95% to 100% of m₁, m₂, m₃, m₄, m₅, m₆, m₇, m₈, m₉, m₁₀, and m₁₁ comprise an adenine-thymine complementary nucleotide base pair. Exemplary examples of one strand of suitable spacer regions in this embodiment are detailed in Table A.

TABLE A

| Spacer Region | |
|---|---|
| Nucleotide Sequence | SEQ. ID.NO. |
| AAGAACAAGAA | 19 |
| AAACAACAAGA | 20 |
| AGAAAGAAAGA | 21 |
| AAAAAAACGCA | 22 |
| AGGCAAAAAAA | 23 |
| CAAAAAAAAGC | 24 |
| AAGAAAAAACC | 25 |
| CAAAAAACGAA | 26 |
| GAAAAAAAACG | 27 |
| CGGAAAAAAAA | 28 |
| ATTATGATCAT | 29 |
| AAATTTGGAAA | 30 |
| TATATATATGC | 31 |
| TTTCAAACTTT | 32 |
| CGATTATTATT | 33 |
| AAAGACAAAAA | 34 |
| TTTCTGTTTTT | 35 |
| GCATATATATA | 36 |
| TTTTTAAAACC | 37 |
| AAAAGCTTTTT | 38 |
| AAAAAAAAAAG | 39 |
| CATATATATAT | 40 |
| TTTTTTTTTTG | 41 |
| ATTATTATTAC | 42 |
| TAATAATATTG | 43 |
| GAAAAATTTTT | 44 |
| ATTTTCAAAAA | 45 |
| TGAAAATTTAA | 46 |
| AATTAATCTAA | 47 |
| TTAGATTAATA | 48 |
| AAAAAAAAAAA | 49 |
| TTTTTTTTTTT | 50 |
| TATATATATAT | 51 |
| ATATATATATA | 52 |
| ATTTATTTATT | 53 |
| TAATAATAATA | 54 |
| ATTAATTAATT | 55 |
| TAATTAATTAA | 56 |
| ATAAAATTTTA | 57 |
| TATTTTAAAAT | 58 |

In yet another embodiment for mutant loxP sites having formula (II), the spacer region will share substantial sequence identity with the wild-type loxP spacer region, but will contain three additional nucleotide base pairs. In one alternative of this embodiment, the spacer region comprising m₁, m₂, m₃, m₄, m₅, m₆, m₇, m₈, m₉, m₁₀, and m₁₁ will have a nucleotide sequence approximately 50% to 75% identical to the wild-type loxP spacer region. In yet another alternative of this embodiment, the spacer region comprising m₁, m₂, m₃, m₄, m₅, m₆, m₇, m₈, m₉, m₁₀, and m₁₁ will have a nucleotide sequence approximately 75% to 80% identical to the wild-type loxP spacer region. In still another alternative of this embodiment, the spacer region comprising m₁, m₂, m₃, m₄, m₅, m₆, m₇, m₈, m₉, m₁₀, and m₁₁ will have a nucleotide sequence approximately 80% to 85% identical to the wild-type loxP spacer region. In yet another embodiment, the spacer region comprising m₁, m₂, m₃, m₄, m₅, m₆, m₇, m₈, m₉, m₁₀, and m₁₁ will have a nucleotide sequence approximately 85% to 90% identical to the wild-type loxP spacer region. In a further alternative of this embodiment, the spacer region comprising m₁, m₂, m₃, m₄, m₅, m₆, m₇, m₈, m₉, m₁₀, and m₁₁ will have a nucleotide sequence greater than 90% identical to the wild-type loxP spacer region. Exemplary examples of one strand of suitable spacer regions in this embodiment are detailed in Table B.

TABLE B

| Spacer Region | |
|---|---|
| Nucleotide Sequence | SEQ. ID NO. |
| ATGTATTTTTA | 59 |
| TGTATAAAAAT | 60 |
| ATTGTATGTTA | 61 |
| TATGCATATAT | 62 |
| AATAATTATGC | 63 |
| TTTATGTAAAA | 64 |
| ATATGTATATA | 65 |
| ATTAATGTATG | 66 |
| GTATGAAATTA | 67 |
| AATAATGTATT | 68 |
| AAAAATGTATT | 69 |
| TATGTATGTAA | 70 |
| TGTATGCTAAT | 71 |
| TTTATGTATAA | 72 |
| ATATGTATATA | 73 |
| TATGTATGCTA | 74 |
| AATTGTATGCT | 75 |
| TTTATGTATGA | 76 |
| AAAAAATGTAT | 77 |
| ATGTATATTAT | 78 |
| TTTGTATGCTT | 79 |
| AAATGTATGCA | 80 |
| ATATTGTATGC | 81 |
| TGTATGCAATT | 82 |
| ATGTATGTTAA | 83 |
| TATGTATGTAA | 84 |
| AAATGTATGAT | 85 |
| TTAATGTATGT | 86 |
| ATATATGTATG | 87 |
| TATGTATGCAT | 88 |
| ATGCATGTATT | 89 |
| GTATGCATAAA | 90 |
| TTACGTATGTA | 91 |
| ATATGCATGAT | 92 |
| TTTGTATGCAT | 93 |
| AAATGTATGCA | 94 |
| TTGTATGCAAA | 95 |
| TATATGTATGC | 96 |
| ACGTATGTATA | 97 |
| CGTATGTAATA | 98 |

In an exemplary embodiment, the mutant lox site is a loxP site represented by formula (III): 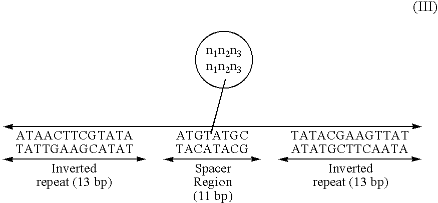
wherein:
n₁, n₂, and n₃ are independently a complementary base pair wherein the nitrogenous base is a purine or pyrimidine.

Suitable mutant loxP sites having formula (III) comprise the two 13 base pair inverted repeats of the wild-type loxP separated by an eleven base pair spacer region. The spacer region for mutant loxP sites having formula (III) comprise the eight-nucleotide complementary base pairs of the wild-type loxP site with three additional complementary base pair additions.

For mutant loxP sites having formula (III), the three additional nucleotide base pairs are generally selected so as to include nitrogenous bases from either the purine or the pyrimidine chemical classes. But this choice is also not a critical feature of the invention to the extent that the base pairs are complementary. For example, the three additional nucleotides may be all purines or all pyrimidines. The three additional nucleotides may be two purines and one pyrimidine. Alternatively, the three nucleotides may include one purine and two pyrimidines. Suitable purines include adenine, guanine, hypoxanthine and xanthine. In exemplary embodiments, the purine will be either adenine or guanine. Suitable pyrimidines include cytosine, uracil or thymine.

In one embodiment for mutant loxP sites having formula (III), n₁, n₂ and n₃ are independently selected from the group consisting of adenosine 5′-monophosphate, thymidine 5′-monophosphate, guanosine 5′-monophosphate and cytidine 5′-monophosphate. In one alternative of this embodiment, n₁ and n₂ are adenosine 5′-monophosphates and n₃ is guanosine 5′-5 monophosphate. In another alternative embodiment, n₁ and n₂ are adenosine 5′-monophosphates and n₃ is cytidine 5′-monophosphate. In another alternative embodiment, n₁ and n₂ are thymidine 5′-monophosphates and n₃ is guanosine 5′-monophosphate. In an additional alternative embodiment, n₁ and n₂ are thymidine 5′-monophosphates and n₃ is cytidine 5′-monophosphate. In yet an additional alternative embodiment, n₁ is thymidine 5′-monophosphate, n₂ is adenosine 5′-monophosphate and n₃ is cytidine 5′-monophosphate. In an additional alternative embodiment, n₁ is thymidine 5′-monophosphate, n₂ is adenosine 5′-monophosphate, and n₃ is guanosine 5′-monophosphate. In still a further embodiment, n₁ and n₂ are guanosine 5′-monophosphates and n₃ is adenosine 5′-monophosphate. In yet another alternative embodiment, n₁ and n₂ are guanosine 5′-monophosphates and n₃ is thymidine 5′-monophosphate. In an additional alternative embodiment, n₁ and n₂ are cytidine 5′-monophosphates, and n₃ is adenosine 5′-monophosphate. In still another alternative embodiment, n₁ and n₂ are cytidine 5′-monophosphates, and n₃ is thymidine 5′-monophosphate. In still another alternative embodiment, n₁ is thymidine 5′-monophosphate, n₂ is guanosine 5′-monophosphate, and n₃ is cytidine 5′-monophosphate. In another alternative embodiment, n₁ is adenosine 5′-monophosphate, n₂ is guanosine 5′-monophosphate, and n₃ is cytidine 5′-monophosphate.

Yet another embodiment encompasses mutant loxP sites having formula (III), wherein n₁, n₂ and n₃ are independently selected from the group consisting of guanosine 5′-monophosphate and cytidine 5′-monophosphate. In one alternative of this embodiment, n₁ and n₂ are guanosine 5′-monophosphates and n₃ is cytidine 5′-monophosphate. In a further alternative of this embodiment, n₁ and n₂ are cytidine 5′-monophosphates and n₃ is guanosine 5′-monophosphate. In still another alternative of this embodiment, n₁, n₂ and n₃ are all guanosine 5′-monophosphate. In yet another alternative of this embodiment, n₁, n₂ and n₃ are all cytidine 5′-monophosphate.

In an exemplary embodiment for mutant loxP sites having formula (III), n₁, n₂ and n₃ are independently selected from the group consisting of adenosine 5′-monophosphate and thymidine 5′-monophosphate. In another alternative of this embodiment, n₁ and n₂ are thymidine 5′-monophosphate and n₃ is adenosine 5′-monophosphate. In yet another alternative of this embodiment, n₁ and n₂ are adenosine 5′-monophosphate and n₃ is thymidine 5′-monophosphate. In yet another alternative of this embodiment, n₁, n₂ and n₃ are all adenosine 5′-monophosphate. In still another alternative embodiment, n₁, n₂ and n₃ are all thymidine 5′-monophosphate.

In any of the embodiments for mutant loxP sites having formula (III) described herein, the choice of placement of the three additional nucleotide base pairs within the spacer region is not a critical aspect of the invention. Typically, the three additional nucleotide base pairs can be inserted before or after any single nucleotide within the wild-type spacer region. In one embodiment, the three additional nucleotide base pairs are inserted within the wild-type spacer region consecutively so that the nucleotide base pairs appear within the spacer region one right after another. In an alternative embodiment, the three additional nucleotide base pairs are inserted within the spacer region so that two of the nucleotides are inserted consecutively, i.e., one right after the other, and the other nucleotide base pair is inserted before or after any single nucleotide in the wild-type spacer region, but not next to the two other inserted nucleotide base pairs. In a further alternative embodiment, the three additional nucleotide base pairs are singly inserted within the wild-type spacer region so that none of the inserted nucleotide base pairs are next to one and other. Exemplary non-limiting examples of one strand of suitable spacer regions for mutant loxP sites having formula (III) are shown in Table C.

TABLE C

| | |
|---|---|
| Spacer Region | |
| Nucleotide Sequence | SEQ. ID NO. |

| | |
|---|---|
| AGTATGTATGC | 99 |
| ATGTATGCGAT | 100 |
| AATGTTATGGC | 101 |
| ATGTGATATGC | 102 |
| GCGTATGTATA | 103 |
| CGTATGTAGTA | 104 |
| GTATTGGCAGT | 105 |
| TATGCATGTAG | 106 |
| TGTAAGTTAGC | 107 |
| TTAGCATGTAG | 108 |
| TCAATGTATGC | 109 |
| CGTATGTATCA | 110 |
| ATGCTACTGTA | 111 |
| TGTAACTTGCT | 112 |
| GTAATGCCATT | 113 |
| ATTAGCATGTC | 114 |
| CATCGTATGTA | 115 |
| ATGTTAACTGC | 116 |
| ATTGTATTGCC | 117 |
| GCATCTAGTAT | 118 |
| TATATGTATGC | 119 |
| CGTATGTAATT | 120 |
| ATAGTTATTGC | 121 |
| TTGTAATGCAT | 122 |
| ATGTTATATGC | 123 |
| ATTATGCATGT | 124 |
| GTATGCATTAT | 125 |
| ATGTCAATTGT | 126 |
| AATGTTATTGC | 127 |
| CATGTTATATG | 128 |
| TAAATGTATGC | 129 |
| ATGTATGCTAA | 130 |
| CGTAAATTGTA | 131 |
| TGCATAGTATA | 132 |
| AATGTTATAGC | 133 |
| TATAGCTATAG | 134 |
| AATCGTATGTA | 135 |
| ATGTATGCAAT | 136 |
| ATTGCAATGAT | 137 |
| TGTAATATGCA | 138 |

In yet another preferred embodiment, the mutant lox site is a loxP site represented by the following formula (IV) 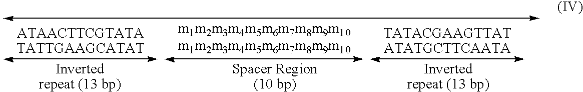
wherein:
m₁, m₂, m₃, m₄, m₅, m₆, m₇, m₈, m₉, and m₁₀ together comprise the spacer region and are independently a complementary nucleotide base pair wherein the nitrogenous base is a purine or a pyrimidine.

In each embodiment for mutant loxP sites having formula (IV) described herein, the inverted repeat region comprises the two 13 base pair inverted repeats of the wild-type loxP separated by a ten base pair spacer region.

Alternatively, the spacer region may include a number of different nucleotide base pair sequences to the extent that the sequence selected can serve as a substrate for the Cre mutant polypeptides of the invention. In one alternative of this embodiment, approximately 75% of m₁, m₂, m₃, m₄, m₅, m₆, m₇, m₈, m₉, and m₁₀ comprise an adenine-thymine complementary nucleotide base pair. In another alternative of this embodiment, approximately 75% to 80% of m₁, m₂, m₃, m₄, m₅, m₆, m₇, m₈, m₉, and m₁₀ comprise an adenine-thymine complementary nucleotide base pair. In still another alternative embodiment, approximately 80% to 85% of m₁, m₂, m₃, m₄, m₅, m₆, m₇, m₈, m₉, and m₁₀ comprise an adenine-thymine complementary nucleotide base pair. In yet another of alternative embodiment, approximately 85% to 90% of m₁, m₂, m₃, m₄, m₅, m₆, m₇, m₈, m₉, and m₁₀ comprise an adenine-thymine complementary nucleotide base pair. In a further alternative of this embodiment, approximately 90% to 95% of m₁, m₂, m₃, m₄, m₅, m₆, m₇, m₈, m₉, and m₁₀₀ comprise an adenine-thymine complementary nucleotide base pair. In yet another alternative embodiment, 95% to 100% of m₁, m₂, m₃, m₄, m₅, m₆, m₇, m₈, m₉, and m₁₀ comprise an adenine-thymine complementary nucleotide base pair. Exemplary examples of one strand of suitable spacer regions in this embodiment are detailed in Table D.

TABLE D

| Spacer Region | |
|---|---|
| Nucleotide Sequence | SEQ. ID NO. |
| TAACTATGAC | 151 |
| AATGATACTG | 152 |
| GGATTATAAC | 153 |
| TACACTGTTA | 154 |
| AAAGCGTTTT | 155 |
| TTTGGGAAAA | 156 |
| CATATATACC | 157 |
| GCTAATTAAC | 158 |
| TCGAATTATC | 159 |
| ATAGGACTTA | 160 |
| AAGTATTGAT | 161 |
| TCAATGATAT | 162 |
| GTTTATAAAG | 163 |
| CTATATATAC | 164 |
| ATATACCTAT | 165 |
| TATTTGGAAT | 166 |
| AAGAAATTCA | 167 |
| TTAACTTCTT | 168 |
| AATGAAGATA | 169 |
| TCTTTTATGA | 170 |
| GATATATATA | 171 |
| CTATATATAT | 172 |
| TTTTTTTTTG | 173 |
| AAAAAAAAAC | 174 |
| TTAATGTAAT | 175 |
| AATTACATTA | 176 |
| TTGATTTATA | 177 |
| AATAATACAT | 178 |
| TTTAGAATAT | 179 |
| AAATTTTACT | 180 |
| AAAAAAAAAA | 181 |
| TATATATATA | 182 |
| ATATATATAT | 183 |
| AATTAATTAA | 184 |
| TTAATTAATT | 185 |
| TTTAAAAATT | 186 |
| TTTTTTTTTT | 187 |
| TTAATTAAAA | 188 |
| AATTTTAAAA | 189 |
| AAATAATTTA | 190 |

In yet another embodiment for mutant loxP sites having formula (IV), the spacer region will share substantial sequence identity with the wild-type loxP spacer region, but will contain two additional nucleotide base pairs. In one alternative of this embodiment, the spacer region comprising m₁, m₂, m₃, m₄, m₅, m₆, m₇, m₈, m₉ and m₁₀ will have a nucleotide sequence approximately 50% to 75% identical to the wild-type loxP spacer region. In yet another alternative of this embodiment, the spacer region comprising m₁, m₂, m₃, m₄, m₅, m₆, m₇, m₈, m₉ and m₁₀ will have a nucleotide sequence approximately 75% to 80% identical to the wild-type loxP spacer region. In still another alternative of this embodiment, the spacer region comprising m₁, m₂, m₃, m₄, m₅, m₆, m₇, m₈, m₉ and m₁₀ will have a nucleotide sequence approximately 80% to 85% identical to the wild-type loxP spacer region. In yet another embodiment, the spacer region comprising m₁, m₂, m₃, m₄, m₅, m₆, m₇, m₈, m₉ and m₁₀ will have a nucleotide sequence approximately 85% to 90% identical to the wild-type loxP spacer region. In a further alternative of this embodiment, the spacer region comprising m₁, m₂, m₃, m₄, m₅, m₆, m₇, m₈, m₉ and m₁₀ will have a nucleotide sequence greater than 90% identical to the wild-type loxP spacer region. Exemplary examples of one strand of suitable spacer regions in this embodiment are detailed in Table E.

TABLE E

| Spacer Region | |
|---|---|
| Nucleotide Sequence | SEQ. ID NO. |
| ATTTGATTAA | 191 |
| AAGATATATG | 192 |
| CGTTAATTGT | 193 |
| TGTAAGATCT | 194 |
| ACAGTTTAAA | 195 |
| CTGATTAATG | 196 |
| TTAATATGGC | 197 |
| TGCGTAATTT | 198 |
| ACAAAAATGG | 199 |
| CAGGTTTTTT | 200 |
| TAGTATGCAT | 201 |
| CAAGTATTTG | 202 |
| ATGTTTTACG | 203 |
| TATACGTAGT | 204 |
| GTATGCAATT | 205 |
| TGTTCATTTG | 206 |
| CGAAGAATTA | 207 |
| AAAGTAGCAT | 208 |
| TTTATGTGCA | 209 |
| ATATATGCGA | 210 |
| GTATTATGCA | 211 |
| ATGCATAATG | 212 |
| AAATGCGTAA | 213 |
| TTCGTATGTT | 214 |
| GATACATGAT | 215 |
| CGGATATATT | 216 |
| TTAAAAGTGC | 217 |
| ATGCGTTTTA | 218 |
| TATTGGATAC | 219 |
| TGTTATTCGA | 220 |
| AATGTATGCT | 221 |
| ATGCTAATGT | 222 |
| GCATATTTAG | 223 |
| GAATGTATAC | 224 |
| AATTCGTATG | 225 |
| CTTTTAGATG | 226 |
| ATAACGAGTT | 227 |
| TCGTATGTAA | 228 |
| ATGAGTTTAC | 229 |
| TGCATTGTAA | 230 |

In an exemplary embodiment, the mutant lox site is a loxP site represented by formula (V): 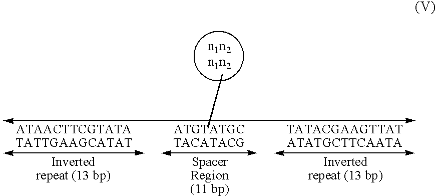
wherein:
n₁, and n₂ are independently a complementary base pair wherein the nitrogenous base is a purine or pyrimidine.

Suitable mutant loxP sites having formula (V) comprise the two 13 base pair inverted repeats of the wild-type loxP separated by a ten base pair spacer region. The spacer region for mutant loxP sites having formula (V) comprise the eight-nucleotide complementary base pairs of the wild-type loxP site with two additional complementary base pair additions.

For mutant loxP sites having formula (V), the two additional nucleotide base pairs are generally selected so as to include nitrogenous bases from either the purine or the pyrimidine chemical classes. But this choice is generally not a critical feature of the invention to the extent that the base pairs are complementary. For example, the two additional nucleotides may be all purines or all pyrimidines. The two additional nucleotides may be one purines and one pyrimidine. Suitable purines include adenine, guanine, hypoxanthine and xanthine. In exemplary embodiments, the purine will be either adenine or guanine. Suitable pyrimidines include cytosine, uracil or thymine.

In one embodiment for mutant loxP sites having formula (V), n₁ and n₂ are independently selected from the group consisting of adenosine 5′-monophosphate, thymidine 5′-monophosphate, guanosine 5′-monophosphate and cytidine 5′-monophosphate. In one alternative of this embodiment, n₁ and n₂ are adenosine 5′-monophosphates. In another alternative embodiment, n₁ and n₂ are cytidine 5′-monophosphate. In another alternative embodiment, n₁ and n₂ are thymidine 5′-monophosphates. In an additional alternative embodiment, n₁ and n₂ are guanosine 5′-monophosphate. In a further embodiment, n₁ is adenosine 5′-monophosphate and n₂ is thymidine 5′-monophosphates. In still another embodiment, n₁ is adenosine 5′-monophosphate and n₂ is guanosine 5′-monophosphate. In yet another embodiment, n₁ is adenosine 5′-monophosphate and n₂ is cytidine 5′-monophosphate. In yet an additional alternative embodiment, n₁ is thymidine 5′-monophosphate and n₂ is cytidine 5′-monophosphate. In an additional alternative embodiment, n₁ is thymidine 5′-monophosphate and n₂ is guanosine 5′-monophosphate. In still another embodiment, n₁ is guanosine 5′-monophosphate and n₂ is cytidine 5′-monophosphate.

In any of the embodiments for mutant loxP sites having formula (V) described herein, the choice of placement of the two additional nucleotide base pairs within the spacer region is not generally a critical aspect of the invention. Typically, the two additional nucleotide base pairs can be inserted before or after any single nucleotide within the wild-type spacer region. In one embodiment, the two additional nucleotide base pairs are inserted within the wild-type spacer region consecutively so that the nucleotide base pairs appear within the spacer region one right after another. In an alternative embodiment, the two additional nucleotide base pairs are singly inserted within the wild-type spacer region so that none of the inserted nucleotide base pairs are next to one and other. Exemplary non-limiting examples of one strand of suitable spacer regions for mutant loxP sites having formula (V) are shown in Table F.

TABLE F

| Spacer Region | |
|---|---|
| Nucleotide Sequence | SEQ. ID NO. |
| AATGATATGC | 231 |
| CGTATAAGTA | 232 |
| TATGCATGAA | 233 |
| GTAATAGCAT | 234 |
| ACGTAATAGT | 235 |
| TAAATGTACG | 236 |
| TATGCAAATG | 237 |
| AGTATAGCTA | 238 |
| GTAAATGCAT | 239 |
| ATGCATAAGT | 240 |
| ATGTATGCTT | 241 |
| TTCGTATGTA | 242 |
| TGTATGCATT | 243 |
| GTTATTGCAT | 244 |
| ATGCTATTGT | 245 |
| CGTTATGTTA | 246 |
| TATTGTATGC | 247 |
| ATGCATTTTG | 248 |
| AACTTGTTCG | 249 |
| GGCAATTTTT | 250 |
| GCTTATAATG | 251 |
| AGTGCTTAAT | 252 |
| ATATTATGGC | 253 |
| TTATGTGACA | 254 |
| CATGTGATTT | 255 |
| TAGTACTTAG | 256 |
| GGATCTTTAA | 257 |
| ATTGTGTATC | 258 |
| TTCTAATAGG | 259 |
| CATGATGTTA | 260 |
| TAGGCATGTA | 261 |
| ACTTGTCTAG | 262 |
| CAGTTTGACG | 263 |
| CGTAGGACTT | 264 |
| AATGTCTGAG | 265 |
| TCAACTGTGT | 266 |
| GGCTCGTTAA | 267 |
| CATTTAAGGG | 268 |
| ATCGGGTATC | 269 |
| TGGTTAATCC | 270 |

In yet another preferred embodiment, the mutant lox site is a loxP site represented by the following formula (VI) 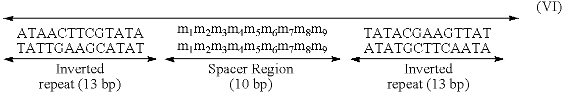
wherein:
m₁, m₂, m₃, m₄, m₅, m₆, m₇, m₈, and m₉ together comprise the spacer region and are independently a complementary nucleotide base pair wherein the nitrogenous base is a purine or a pyrimidine.

In each embodiment for mutant loxP sites having formula (VI) described herein, the inverted repeat region comprises the two 13 base pair inverted repeats of the wild-type loxP separated by a nine base pair spacer region.

Alternatively, the spacer region may include a number of different nucleotide base pair sequences to the extent that the sequence selected can serve as a substrate for the Cre mutant polypeptides of the invention. In one alternative of this embodiment, approximately 75% of m₁, m₂, m₃, m₄, m₅, m₆, m₇, m₈, and m₉ comprise an adenine-thymine complementary nucleotide base pair. In another alternative of this embodiment, approximately 75% to 80% of m₁, m₂, m₃, m₄, m₅, m₆, m₇, m₈, and m₉ comprise an adenine-thymine complementary nucleotide base pair. In still another alternative embodiment, approximately 80% to 85% of m₁, m₂, m₃, m₄, m₅, m₆, m₇, m₈, and m₉ comprise an adenine-thymine complementary nucleotide base pair. In yet another of alternative embodiment, approximately 85% to 90% of m₁, m₂, m₃, m₄, m₅, m₆, m₇, m₈, and m₉ comprise an adenine-thymine complementary nucleotide base pair. In a further alternative of this embodiment, approximately 90% to 95% of m₁, m₂, m₃, m₄, m₅, m₆, m₇, m₈, and m₉ comprise an adenine-thymine complementary nucleotide base pair. In yet another alternative embodiment, 95% to 100% of m₁, m₂, m₃, m₄, m₅, m₆, m₇, m₈, and m₉ comprise an adenine-thymine complementary nucleotide base pair. Exemplary examples of one strand of suitable spacer regions in this embodiment are detailed in Table G.

TABLE G

| | |
|---|---|
| Spacer Region | |
| Nucleotide Sequence | SEQ. ID NO. |

| | |
|---|---|
| AGAGATTCT | 271 |
| TATATACGC | 272 |
| GAAATTACG | 273 |
| ATTTCCGAA | 274 |
| CCAATTATA | 275 |
| TTAGGGATT | 276 |
| ATTAAACGG | 277 |
| GCGTTTATT | 278 |
| TTAGCGAAT | 279 |
| CTCTTTATC | 280 |
| AGTGATATA | 281 |
| TACTCATAT | 282 |
| CAAATTTTG | 283 |
| GTTTAAAAC | 284 |
| TATTGCATT | 285 |
| AAACCTTAA | 286 |
| ATTATGGTA | 287 |
| TTGATTACT | 288 |
| ACATTATAG | 289 |
| TTAGCAATA | 290 |
| AAATCTTAT | 291 |
| TTTTTTGTT | 292 |
| ACAAAAAAA | 293 |
| TTATTATGA | 294 |
| AAACATTTT | 295 |
| GTATATATA | 296 |
| ATATTTAAC | 297 |
| TAATTGAAT | 298 |
| ATCATATAT | 299 |
| AAATATACA | 300 |
| AAAATTTTT | 301 |
| TTTTAAAAA | 302 |
| ATATATATA | 303 |
| TATATATAT | 304 |
| ATTTTAAAT | 305 |
| AATTTAAAT | 306 |
| TTTAATTTA | 307 |
| ATTATATAA | 308 |
| TATTATTAT | 309 |
| ATTTTTAAA | 310 |

In yet another embodiment for mutant loxP sites having formula (VI), the spacer region will share substantial sequence identity with the wild-type loxP spacer region, but will contain one additional nucleotide base pair. In one alternative of this embodiment, the spacer region comprising m₁, m₂, m₃, m₄, m₅, m₆, m₇, m₈, and mg will have a nucleotide sequence approximately 50% to 75% identical to the wild-type loxP spacer region. In yet another alternative of this embodiment, the spacer region comprising m₁, m₂, m₃, m₄, m₅, m₆, m₇, m₈, and m₉ will have a nucleotide sequence approximately 75% to 80% identical to the wild-type loxP spacer region. In still another alternative of this embodiment, the spacer region comprising m₁, m₂, m₃, m₄, m₅, m₆, m₇, m₈, and m₉ will have a nucleotide sequence approximately 80% to 85% identical to the wild-type loxP spacer region. In yet another embodiment, the spacer region comprising m₁, m₂, m₃, m₄, m₅, m₆, m₇, m₈, and m₉ will have a nucleotide sequence approximately 85% to 90% identical to the wild-type loxP spacer region. In a further alternative of this embodiment, the spacer region m₁, m₂, m₃, m₄, m₅, m₆, m₇, m₈, and m₉ will have a nucleotide sequence greater than 90% identical to the wild-type loxP spacer region. Exemplary examples of one strand of suitable spacer regions in this embodiment are detailed in Table H.

TABLE H

| Spacer Region | |
|---|---|
| Nucleotide Sequence | SEQ. ID NO. |
| AAGTAGCTT | 311 |
| CGATATATG | 312 |
| TTCGTTGAA | 313 |
| ATATGAATC | 314 |
| GGATCTATA | 315 |
| CTTAATTAG | 316 |
| TTGTCGAAT | 317 |
| TAAAGCGAT | 318 |
| AATTGGAAC | 319 |
| TCAGTAATA | 320 |
| GAAGCTTAT | 321 |
| TAGCTATGA | 322 |
| CTTAAGTAG | 323 |
| TAAGTGACA | 324 |
| AATTAATAC | 325 |
| GTGTCAATT | 326 |
| TTCTATGGA | 327 |
| AATATCGAG | 328 |
| CATATTTAG | 329 |
| TTGATACAA | 330 |
| ACGTTAGTA | 331 |
| TAACGTTGT | 332 |
| CATTATGAG | 333 |
| TTTGTAAAC | 334 |
| GGATCAATT | 335 |
| AGATTTATG | 336 |
| ATTTTTAGC | 337 |
| TTAAAGGAT | 338 |
| CAAAATTGT | 339 |
| TCTTGGTAA | 340 |
| CGATTTGAA | 341 |
| AATCGTTTG | 342 |
| TCTATGTGT | 343 |
| GGTTAAATC | 344 |
| AACTGTGTA | 345 |
| TTTGTACAG | 346 |
| CGGAAATTT | 347 |
| ATCTTGGAT | 348 |
| TATTCGGAA | 349 |
| AAGTGACTT | 350 |

In an exemplary embodiment, the mutant lox site is a loxP site represented by formula (VII): 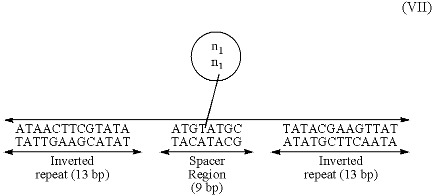
wherein:
n₁ is independently a complementary base pair wherein the nitrogenous base is a purine or pyrimidine.

Suitable mutant loxP sites having formula (VII) comprise the two 13 base pair inverted repeats of the wild-type loxP separated by a nine base pair spacer region. The spacer region for mutant loxP sites having formula (VII) comprise the eight-nucleotide complementary base pairs of the wild-type loxP site with one additional complementary base pair addition.

For mutant loxP sites having formula (VII), the one additional nucleotide base pair is generally selected so as to include nitrogenous bases from either the purine or the pyrimidine chemical classes. But this choice is generally not a critical feature of the invention to the extent that the base pair is complementary. For example, the additional nucleotide may be a purine or a pyrimidine. Suitable purines include adenine, guanine, hypoxanthine and xanthine. In exemplary embodiments, the purine will be either adenine or guanine. Suitable pyrimidines include cytosine, uracil or thymine.

In one embodiment for mutant loxP sites having formula (VII), n₁ is adenosine 5′-monophosphate. In one alternative of this embodiment, n₁ is cytidine 5′-monophosphate. In another alternative embodiment, n₁ is thymidine 5′-monophosphates. In an additional alternative embodiment, n₁ is guanosine 5′-monophosphate.

In any of the embodiments for mutant loxP sites having formula (VII) described herein, the choice of placement of the additional nucleotide base pair within the spacer region is not generally a critical aspect of the invention. Typically, the additional nucleotide base pair can be inserted before or after any single nucleotide within the wild-type spacer region. Exemplary non-limiting examples of one strand of suitable spacer regions for mutant loxP sites having formula (VII) are shown in Table I.

TABLE I

| Spacer Region | |
|---|---|
| Nucleotide Sequence | SEQ. ID NO. |
| CATGATTAG | 351 |
| GCGTTTAAA | 352 |
| AAATCGGTT | 353 |
| TAAGTATGC | 354 |
| TTTCAGAGA | 355 |
| AGCTGAATT | 356 |
| CTTAATGGA | 357 |
| GGTAAATCT | 358 |
| ACGTATTAG | 359 |
| AGATTTAGC | 360 |
| TATCTGTAG | 361 |
| CTGGATATT | 362 |
| TGTATTCGA | 363 |
| ATATGCTTG | 364 |
| GATTTTGAC | 365 |
| AAGTCGTTT | 366 |
| GTACTTTGA | 367 |
| TCATTGTGA | 368 |
| ATTAGCGTT | 369 |
| TGTGTTCAA | 370 |
| TTGGACAGT | 371 |
| GATTTGGAC | 372 |
| AGCATGTTG | 373 |
| CTGGGTATA | 374 |
| AATTGTCGG | 375 |
| GACATGTTG | 376 |
| GGTTTCGAA | 377 |
| AAGGTTTGC | 378 |
| CTGTAAGTG | 379 |
| TGTAGCGAT | 380 |
| CTGATTAGC | 381 |
| TCATGGTCA | 382 |
| GGCATACTT | 383 |
| ATTCACTGG | 384 |
| TGCGCATTA | 385 |
| AGGCTCTAT | 386 |
| GTCTTACAG | 387 |
| ACTTGGTCA | 388 |
| CGGATTTAC | 389 |
| GTCATCGTA | 390 |

The mutant lox sites may be produced by a number of methods generally known in the art or as described in the examples herein. For example, lox sites can be produced by a variety of synthetic techniques that are known in the art, such as the synthetic techniques for producing lox sites described by Ito et al. (1982) Nuc. Acid Res., 10: 1755; and Ogilvie et al., (1981) Science, 214: 270.

Vectors

In order to express a biologically active Cre mutant polypeptide, the nucleotide sequences encoding such polypeptides may be inserted into an appropriate expression vector. Non limiting examples of suitable expression vector are described in the examples. An “appropriate vector” is typically a vector that contains the necessary elements for transcriptional and translational control of the inserted coding sequence in a suitable host. These elements generally will include regulatory sequences, such as enhancers, constitutive and inducible promoters, and 5′ and 3′ untranslated regions in the vector and polynucleotide sequences encoding Cre mutant polypeptides of the invention. Such elements may vary in their strength and specificity. Specific initiation signals may also be used to achieve more efficient translation of nucleotide sequences encoding Cre mutant polypeptides. These signals, for example, include the ATG initiation codon and adjacent sequences (e.g. the Kozak sequence). In cases where nucleotide sequences encoding the subject polypeptide and its initiation codon and upstream regulatory sequences are inserted into the appropriate expression vector, no additional transcriptional or translational control signals may be needed. But in cases where only coding sequence, or a fragment thereof, is inserted, exogenous translational control signals including an in-frame ATG initiation codon should be provided by the vector. Exogenous translational elements and initiation codons may be of various origins, both natural and synthetic. The efficiency of expression may be enhanced by the inclusion of enhancers appropriate for the particular host cell system used (See, e.g., Scharf, D. et al. (1994) Results Probl. Cell Differ. 20:125-162).

Depending upon the embodiment, either eukaryotic or prokaryotic vectors may be used. Suitable eukaryotic vectors that may be used include MSCV, Harvey murine sarcoma virus, pFastBac, pFastBac HT, pFastBac DUAL, pSFV, pTet-Splice, pEUK-C₁, pPUR, pMAM, pMAMneo, pBI101, pBI121, pDR2, pCMVEBNA, YACneo, pSVK3, pSVL, pMSG, pCH110, pKK232-8, p3′SS, pBlueBacIII, pCDM8, pcDNA1, pZeoSV, pcDNA3, pREP4, pCEP4, and pEBVHis vectors. The MSCV or Harvey murine sarcoma virus is preferred. Suitable prokaryotic vectors that can be used in the present invention include pET, pET28, pcDNA3.11V5-His-TOPO, pCS2+, pcDNA II, pSL301, pSE280, pSE380, pSE420, pTrcHis, pRSET, pGEMEX-1, pGEMEX-2, pTrc99A, pKK223-3, pGEX, pEZZ18, pRIT2T, pMC1871, pKK233-2, pKK38801, and pProEx-HT vectors.

Methods that are well known to those skilled in the art may be used to construct expression vectors containing sequences encoding the Cre mutant polypeptide and appropriate transcriptional and translational control elements. These methods include, for example, in vitro recombinant DNA techniques, synthetic techniques, and in vivo genetic recombination. (See, e.g., Sambrook, J. et al. (1989) Molecular Cloning, A Laboratory Manual, Cold Spring Harbor Press, Plainview N.Y., ch. 4, 8, and 16-17; Ausubel, F. M. et al. (1995) Current Protocols in Molecular Biology, John Wiley & Sons, New York N.Y., ch. 9, 13, and 16).

It is also contemplated that a variety of expression vector/host systems may be utilized to contain and express nucleotide sequences encoding polypeptides of the invention. By way of non limiting example, these include microorganisms such as bacteria transformed with recombinant bacteriophage, plasmid, or cosmid DNA expression vectors; yeast transformed with yeast expression vectors; insect cell systems infected with viral expression vectors (e.g., baculovirus); plant cell systems transformed with viral expression vectors (e.g., cauliflower mosaic virus, CaMV, or tobacco mosaic virus, TMV) or with bacterial expression vectors (e.g., Ti or pBR322 plasmids); or animal cell systems. (See, e.g., Sambrook, supra; Ausubel, supra; Van Heeke, G. and S. M. Schuster (1989) J. Biol. Chem. 264:5503-5509; Engelhard, E. K. et al. (1994) Proc. Natl. Acad. Sci. USA 91:3224-3227; Sandig, V. et al. (1996) Hum. Gene Ther. 7:1937-1945; Takamatsu, N. (1987) EMBO J. 6:307-311; The McGraw Hill Yearbook of Science and Technology (1992) McGraw Hill, New York N.Y., pp. 191-196; Logan, J. and T. Shenk (1984) Proc. Natl. Acad. Sci. USA 81:3655-3659; and Harrington, J. J. et al. (1997) Nat. Genet. 15:345-355). In additional embodiments, expression vectors derived from retroviruses, adenoviruses, or herpes or vaccinia viruses, or from various bacterial plasmids, may be used for delivery of nucleotide sequences to the targeted organ, tissue, or cell population. (See, e.g., Di Nicola, M. et al. (1998) Cancer Gen. Ther. 5(6):350-356; Yu, M. et al. (1993) Proc. Natl. Acad. Sci. USA 90(13):6340-6344; Buller, R. M. et al. (1985) Nature 317(6040):813-815; McGregor, D. P. et al. (1994) Mol. Immunol. 31(3):219-226; and Verma, L M. and N. Somia (1997) Nature 389:239-242).

In one aspect of the invention, accordingly, a bacterial expression system is employed. In bacterial systems, a number of cloning and expression vectors may be selected depending upon the use intended for nucleotide sequence. For example, routine cloning, subcloning, and propagation of nucleotide sequences can be achieved using a multifunctional E. coli vector such as PBLUESCRIPT (Stratagene, La Jolla Calif.) or PSPORT1 plasmid (Life Technologies). Ligation of nucleotide sequences encoding Cre mutant polypeptides into the vector's multiple cloning sites disrupts the lacZ gene, advantageously allowing a colorimetric screening procedure for identification of transformed bacteria containing the subject recombinant molecule. When large quantities of polypeptide are needed, vectors that direct high level expression of Cre mutant polypeptides may be used. For example, vectors containing the strong, inducible SP6 or T7 bacteriophage promoter may be used for this embodiment.

A further aspect of the invention encompasses the use of yeast expression systems. In this embodiment, a number of vectors containing constitutive or inducible promoters, such as alpha factor, alcohol oxidase, and PGH promoters, may be used in the yeast Saccharomyces cerevisiae or Pichia pastoris. In addition, such vectors advantageously direct either the secretion or intracellular retention of expressed proteins and enable integration of foreign sequences into the host genome for stable propagation. (See, e.g., Ausubel, 1995, supra; Bitter, G. A. et al. (1987) Methods Enzymol. 153:516-544; and Scorer, C. A. et al. (1994) Bio/Technology 12:181-184).

In a further aspect of the invention, a plant system may also be used for expression of Cre mutant polypeptides. Transcription of nucleotide sequences encoding the subject polypeptide may be driven by viral promoters, e.g., the 35S and 19S promoters of CaMV used alone or in combination with the omega leader sequence from TMV (Takamatsu, N. (1987) EMBO J. 6:307-311). Alternatively, plant promoters such as the small subunit of RUBISCO or heat shock promoters may be used. (See, e.g., Coruzzi, G. et al. (1984) EMBO J. 3:1671-1680; Broglie, R. et al. (1984) Science 224:838-843; and Winter, J. et al. (1991) Results Probl. Cell Differ. 17:85-105). These constructs can be introduced into plant cells by direct DNA transformation or pathogen-mediated transfection. (See, e.g., The McGraw Hill Yearbook of Science and Technology (1992) McGraw Hill, New York N.Y., pp. 191-196).

An additional aspect of the invention contemplates the use of a mammalian system for expression of Cre mutant polypeptides. In mammalian cells, a number of viral-based expression systems may be utilized. For example, in cases where an adenovirus is used as an expression vector, nucleotide sequences may be ligated into an adenovirus transcription/translation complex consisting of the late promoter and tripartite leader sequence. Insertion in a non-essential E1 or E3 region of the viral genome may be used to obtain infective virus that will express the subject polypeptide in host cells. (See, e.g., Logan, J. and T. Shenk (1984) Proc. Natl. Acad. Sci. USA 81:3655-3659). In addition, transcription enhancers, such as the Rous sarcoma virus (RSV) enhancer, may be used to increase expression in mammalian host cells. SV40 or EBV-based vectors may also be used for high-level protein expression.

Alternatively, human artificial chromosomes (HACs) may also be employed to deliver larger fragments of nucleotide sequence than can be contained in and expressed from a plasmid. HACs of about 6 kb to 10 Mb are constructed and delivered via conventional delivery methods (liposomes, polycationic amino polymers, or vesicles) for therapeutic purposes. (See, e.g., Harrington, J. J. et al. (1997) Nat. Genet. 15:345-355).

For long term production of recombinant proteins in mammalian systems, stable expression of Cre mutant polypeptides in cell lines is preferred. For example, nucleotide sequences encoding Cre mutant polypeptides can be transformed into cell lines using expression vectors that may contain viral origins of replication and/or endogenous expression elements and a selectable marker gene on the same or on a separate vector. Following the introduction of the vector, cells may be allowed to grow for about 1 to 2 days in enriched media before being switched to selective media. The purpose of the selectable marker is to confer resistance to a selective agent, and its presence allows growth and recovery of cells that successfully express the introduced sequences. Resistant clones of stably transformed cells may be propagated using tissue culture techniques appropriate to the cell type.

Any number of selection systems may be used to recover transformed cell lines. These include, but are not limited to, the herpes simplex virus thymidine kinase and adenine phosphoribosyltransferase genes, for use in tk⁻ and apr⁻ cells, respectively. (See, e.g., Wigler, M. et al. (1977) Cell 11:223-232; Lowy, I. et al. (1980) Cell 22:817-823.) Also, antimetabolite, antibiotic, or herbicide resistance can be used as the basis for selection. For example, dhfr confers resistance to methotrexate; neo confers resistance to the aminoglycosides neomycin and G-418; and als and pat confer resistance to chlorsulfuron and phosphinotricin acetyltransferase, respectively. (See, e.g., Wigler, M. et al. (1980) Proc. Natl. Acad. Sci. USA 77:3567-3570; Colbere-Garapin, F. et al. (1981) J. Mol. Biol. 150:1-14). Additional selectable genes have been described, e.g., trpB and hisD, which alter cellular requirements for metabolites. (See, e.g., Hartman, S. C. and R. C. Mulligan (1988) Proc. Natl. Acad. Sci. USA 85:8047-8051). Visible markers, e.g., anthocyanins, green fluorescent proteins (GFP; Clontech), β-glucuronidase and its substrate β-glucuronide, or luciferase and its substrate luciferin may be used. These markers can be used not only to identify transformants, but also to quantify the amount of transient or stable protein expression attributable to a specific vector system. (See, e.g., Rhodes, C. A. (1995) Methods Mol. Biol. 55:121-131).

Although the presence/absence of marker gene expression suggests that the nucleotide sequence of interest is also present, the presence and expression of the gene may need to be confirmed. For example, if the sequence encoding a Cre mutant polypeptide is inserted within a marker gene sequence, transformed cells containing the subject polypeptide can be identified by the absence of marker gene function. Alternatively, a marker gene can be placed in tandem with a sequence encoding a subject polypeptide under the control of a single promoter. Expression of the marker gene in response to induction or selection usually indicates expression of the tandem gene as well.

Generally speaking, host cells that contain the nucleotide sequence encoding Cre mutant polypeptides may be identified by a variety of procedures known to those of skill in the art. These procedures include, but are not limited to, DNA-DNA or DNA-RNA hybridizations, PCR amplification, and protein bioassay or immunoassay techniques that include membrane, solution, or chip based technologies for the detection and/or quantification of nucleic acid or protein sequences.

Host cells transformed with nucleotide sequences encoding Cre mutant polypeptides may be cultured under conditions suitable for the expression and recovery of the protein from cell culture. The protein produced by a transformed cell may be secreted or retained intracellularly depending on the sequence and/or the vector used. As will be understood by those of skill in the art, expression vectors containing the subject nucleotide sequence may be designed to contain signal sequences that direct secretion of the subject polypeptides through a prokaryotic or eukaryotic cell membrane. In addition, a host cell strain may be chosen for its ability to modulate expression of the inserted nucleotide sequences or to process the expressed protein in the desired fashion. Such modifications of the polypeptide include, but are not limited to, acetylation, carboxylation, glycosylation, phosphorylation, lipidation, and acylation. Post-translational processing that cleaves a “prepro” or “pro” form of the protein may also be used to specify protein targeting, folding, and/or activity. Different host cells that have specific cellular machinery and characteristic mechanisms for post-translational activities (e.g., CHO, HeLa, MDCK, HEK293, and W138) are available from the American Type Culture Collection (ATCC, Manassas Va.) and may be chosen to ensure the correct modification and processing of the foreign protein.

Cre/lox Systems

Another aspect of the invention encompasses a Cre/lox system. The system typically comprises any of the Cre mutant polypeptides described above and at least one of any of the mutant lox sites having a spatially extended spacer region as described above. The novel Cre/lox system may be used alone or in combination with other Cre/lox systems currently known in the art. A number of methods utilizing the Cre/lox system of the invention are described in detail below.

In one aspect of the invention, suitable examples of Cre mutant polypeptides and mutant lox sites that may be employed in the Cre/lox system are shown in table J.

TABLE J

| Cre Mutant Polypeptide | Two Mutant lox site having: |
|---|---|
| SEQ. ID. No. 2 | Formula (I) |
| SEQ. ID. No. 3 | Formula (I) |
| SEQ. ID. No. 4 | Formula (I) |
| SEQ. ID. No. 5 | Formula (I) |
| SEQ. ID. No. 2 | Formula (II) |
| SEQ. ID. No. 3 | Formula (II) |
| SEQ. ID. No. 4 | Formula (II) |
| SEQ. ID. No. 5 | Formula (II) |
| SEQ. ID. No. 2 | Formula (III) |
| SEQ. ID. No. 3 | Formula (III) |
| SEQ. ID. No. 4 | Formula (III) |
| SEQ. ID. No. 5 | Formula (III) |
| SEQ. ID. No. 2 | Formula (IV) |
| SEQ. ID. No. 3 | Formula (IV) |
| SEQ. ID. No. 4 | Formula (IV) |
| SEQ. ID. No. 5 | Formula (IV) |
| SEQ. ID. No. 2 | Formula (V) |
| SEQ. ID. No. 3 | Formula (V) |
| SEQ. ID. No. 4 | Formula (V) |
| SEQ. ID. No. 5 | Formula (V) |
| SEQ. ID. No. 2 | Formula (VI) |
| SEQ. ID. No. 3 | Formula (VI) |
| SEQ. ID. No. 4 | Formula (VI) |
| SEQ. ID. No. 5 | Formula (VI) |
| SEQ. ID. No. 2 | Formula (VII) |
| SEQ. ID. No. 3 | Formula (VII) |
| SEQ. ID. No. 4 | Formula (VII) |
| SEQ. ID. No. 5 | Formula (VII) |

In another alternative embodiment, suitable examples of Cre mutant polypeptides and mutant lox sites that may be employed in the Cre/lox system are shown in Table K.

TABLE K

| Cre Mutant Polypeptide | Two Mutant lox sites selected from: |
|---|---|
| SEQ. ID. No. 2 | SEQ. ID. Nos. 19, 20, 21, 22, 23, 24, 25, 26, 27, 28, 29, 30, 31, |
| | 32, 33, 34, 35, 36, 37, 38, 39, 40, 41, 42, 43, 44, 45, 46, 47, 48, |
| | 49, 50, 51, 52, 53, 54, 55, 56, 57, 58, 59, 60, 61, 62, 63, 64, 65, |
| | 66, 67, 68, 69, 70, 71, 72, 73, 74, 75, 76, 77, 78, 79, 80, 81, 82, |
| | 83, 84, 85, 86, 87, 88, 89, 90, 91, 92, 93, 94, 95, 96, 97, 98, 99, |
| | 100, 101, 102, 103, 104 105, 106, 107, 108, 109, 110, 111, 112, |
| | 113, 114, 115, 116, 117, 118, 119, 120, 121, 122, 123, 124, 125, |
| | 126, 127, 128, 129, 130, 131, 132, 133, 134, 135, 136, 137, 138, |
| | 151, 152, 153, 154, 155, 156, 157, 158, 159, 160, 161, 162, 163, |
| | 164, 165, 166, 167, 168, 169, 170, 171, 172, 173, 174, 175, 176, |
| | 177, 178, 179, 180, 180, 182, 183, 184, 185, 186, 187, 188, 189, |
| | 190, 191, 192, 193, 194, 195, 196, 197, 198, 199, 200, 201, 202, |
| | 203, 204, 205, 206, 207, 208, 209, 210, 211, 212, 213, 214, 215, |
| | 216, 217, 218, 219, 220, 221, 222, 223, 224, 225, 226, 227, 228, |
| | 229, 230, 231, 232, 233, 234, 235, 236, 237, 238, 239, 240, 241, |
| | 242, 243, 244, 245, 246, 247, 248, 249, 250, 251, 252, 253, 254, |
| | 255, 256, 257, 258, 259, 260, 261, 262, 263, 264, 265, 266, 267, |
| | 268, 269, 270, 271, 272, 273, 274, 275, 276, 277, 278, 279, 280, |
| | 281, 282, 283, 284, 285, 286, 287, 288, 289, 290, 291, 292, 293, |
| | 294, 295, 296, 297, 298, 299, 300, 301, 302, 303, 304, 305, 306, |
| | 307, 308, 309, 310, 311, 312, 313, 314, 315, 316, 317, 318, 319, |
| | 320, 321, 322, 323, 324, 325, 326, 327, 328, 329, 330, 331, 332, |
| | 333, 334, 335, 336, 337, 338, 339, 340, 341, 342, 343, 344, 345, |
| | 346, 347, 348, 349, 350, 351, 352, 353, 354, 355, 356, 357, 358, |
| | 359, 360, 361, 362, 363, 364, 365, 366, 367, 368, 369, 370, 371, |
| | 372, 373, 374, 375, 376, 377, 378, 379, 380, 381, 382, 383, 384, |
| | 385, 386, 387, 388, 389 and 390. |
| SEQ. ID. No. 3 | SEQ. ID. Nos. 19, 20, 21, 22, 23, 24, 25, 26, 27, 28, 29, 30, 31, |
| | 32, 33, 34, 35, 36, 37, 38, 39, 40, 41, 42, 43, 44, 45, 46, 47, 48, |
| | 49, 50, 51, 52, 53, 54, 55, 56, 57, 58, 59, 60, 61, 62, 63, 64, 65, |
| | 66, 67, 68, 69, 70, 71, 72, 73, 74, 75, 76, 77, 78, 79, 80, 81, 82, |
| | 83, 84, 85, 86, 87, 88, 89, 90, 91, 92, 93, 94, 95, 96, 97, 98, 99, |
| | 100, 101, 102, 103, 104 105, 106, 107, 108, 109, 110, 111, 112, |
| | 113, 114, 115, 116, 117, 118, 119, 120, 121, 122, 123, 124, 125, |
| | 126, 127, 128, 129, 130, 131, 132, 133, 134, 135, 136, 137, 138, |
| | 151, 152, 153, 154, 155, 156, 157, 158, 159, 160, 161, 162, 163, |
| | 164, 165, 166, 167, 168, 169, 170, 171, 172, 173, 174, 175, 176, |
| | 177, 178, 179, 180, 180, 182, 183, 184, 185, 186, 187, 188, 189, |
| | 190, 191, 192, 193, 194, 195, 196, 197, 198, 199, 200, 201, 202, |
| | 203, 204, 205, 206, 207, 208, 209, 210, 211, 212, 213, 214, 215, |
| | 216, 217, 218, 219, 220, 221, 222, 223, 224, 225, 226, 227, 228, |
| | 229, 230, 231, 232, 233, 234, 235, 236, 237, 238, 239, 240, 241, |
| | 242, 243, 244, 245, 246, 247, 248, 249, 250, 251, 252, 253, 254, |
| | 255, 256, 257, 258, 259, 260, 261, 262, 263, 264, 265, 266, 267, |
| | 268, 269, 270, 271, 272, 273, 274, 275, 276, 277, 278, 279, 280, |
| | 281, 282, 283, 284, 285, 286, 287, 288, 289, 290, 291, 292, 293, |
| | 294, 295, 296, 297, 298, 299, 300, 301, 302, 303, 304, 305, 306, |
| | 307, 308, 309, 310, 311, 312, 313, 314, 315, 316, 317, 318, 319, |
| | 320, 321, 322, 323, 324, 325, 326, 327, 328, 329, 330, 331, 332, |
| | 333, 334, 335, 336, 337, 338, 339, 340, 341, 342, 343, 344, 345, |
| | 346, 347, 348, 349, 350, 351, 352, 353, 354, 355, 356, 357, 358, |
| | 359, 360, 361, 362, 363, 364, 365, 366, 367, 368, 369, 370, 371, |
| | 372, 373, 374, 375, 376, 377, 378, 379, 380, 381, 382, 383, 384, |
| | 385, 386, 387, 388, 389 and 390. |
| SEQ. ID. No. 4 | SEQ. ID. Nos. 19, 20, 21, 22, 23, 24, 25, 26, 27, 28, 29, 30, 31, |
| | 32, 33, 34, 35, 36, 37, 38, 39, 40, 41, 42, 43, 44, 45, 46, 47, 48, |
| | 49, 50, 51, 52, 53, 54, 55, 56, 57, 58, 59, 60, 61, 62, 63, 64, 65, |
| | 66, 67, 68, 69, 70, 71, 72, 73, 74, 75, 76, 77, 78, 79, 80, 81, 82, |
| | 83, 84, 85, 86, 87, 88, 89, 90, 91, 92, 93, 94, 95, 96, 97, 98, 99, |
| | 100, 101, 102, 103, 104 105, 106, 107, 108, 109, 110, 111, 112, |
| | 113, 114, 115, 116, 117, 118, 119, 120, 121, 122, 123, 124, 125, |
| | 126, 127, 128, 129, 130, 131, 132, 133, 134, 135, 136, 137, 138, |
| | 151, 152, 153, 154, 155, 156, 157, 158, 159, 160, 161, 162, 163, |
| | 164, 165, 166, 167, 168, 169, 170, 171, 172, 173, 174, 175, 176, |
| | 177, 178, 179, 180, 180, 182, 183, 184, 185, 186, 187, 188, 189, |
| | 190, 191, 192, 193, 194, 195, 196, 197, 198, 199, 200, 201, 202, |
| | 203, 204, 205, 206, 207, 208, 209, 210, 211, 212, 213, 214, 215, |
| | 216, 217, 218, 219, 220, 221, 222, 223, 224, 225, 226, 227, 228, |
| | 229, 230, 231, 232, 233, 234, 235, 236, 237, 238, 239, 240, 241, |
| | 242, 243, 244, 245, 246, 247, 248, 249, 250, 251, 252, 253, 254, |
| | 255, 256, 257, 258, 259, 260, 261, 262, 263, 264, 265, 266, 267, |
| | 268, 269, 270, 271, 272, 273, 274, 275, 276, 277, 278, 279, 280, |
| | 281, 282, 283, 284, 285, 286, 287, 288, 289, 290, 291, 292, 293, |
| | 294, 295, 296, 297, 298, 299, 300, 301, 302, 303, 304, 305, 306, |
| | 307, 308, 309, 310, 311, 312, 313, 314, 315, 316, 317, 318, 319, |
| | 320, 321, 322, 323, 324, 325, 326, 327, 328, 329, 330, 331, 332, |
| | 333, 334, 335, 336, 337, 338, 339, 340, 341, 342, 343, 344, 345, |
| | 346, 347, 348, 349, 350, 351, 352, 353, 354, 355, 356, 357, 358, |
| | 359, 360, 361, 362, 363, 364, 365, 366, 367, 368, 369, 370, 371, |
| | 372, 373, 374, 375, 376, 377, 378, 379, 380, 381, 382, 383, 384, |
| | 385, 386, 387, 388, 389 and 390. |
| SEQ. ID. No. 5 | SEQ. ID. Nos. 19, 20, 21, 22, 23, 24, 25, 26, 27, 28, 29, 30, 31, |
| | 32, 33, 34, 35, 36, 37, 38, 39, 40, 41, 42, 43, 44, 45, 46, 47, 48, |
| | 49, 50, 51, 52, 53, 54, 55, 56, 57, 58, 59, 60, 61, 62, 63, 64, 65, |
| | 66, 67, 68, 69, 70, 71, 72, 73, 74, 75, 76, 77, 78, 79, 80, 81, 82, |
| | 83, 84, 85, 86, 87, 88, 89, 90, 91, 92, 93, 94, 95, 96, 97, 98, 99, |
| | 100, 101, 102, 103, 104 105, 106, 107, 108, 109, 110, 111, 112, |
| | 113, 114, 115, 116, 117, 118, 119, 120, 121, 122, 123, 124, 125, |
| | 126, 127, 128, 129, 130, 131, 132, 133, 134, 135, 136, 137, 138, |
| | 151, 152, 153, 154, 155, 156, 157, 158, 159, 160, 161, 162, 163, |
| | 164, 165, 166, 167, 168, 169, 170, 171, 172, 173, 174, 175, 176, |
| | 177, 178, 179, 180, 180, 182, 183, 184, 185, 186, 187, 188, 189, |
| | 190, 191, 192, 193, 194, 195, 196, 197, 198, 199, 200, 201, 202, |
| | 203, 204, 205, 206, 207, 208, 209, 210, 211, 212, 213, 214, 215, |
| | 216, 217, 218, 219, 220, 221, 222, 223, 224, 225, 226, 227, 228, |
| | 229, 230, 231, 232, 233, 234, 235, 236, 237, 238, 239, 240, 241, |
| | 242, 243, 244, 245, 246, 247, 248, 249, 250, 251, 252, 253, 254, |
| | 255, 256, 257, 258, 259, 260, 261, 262, 263, 264, 265, 266, 267, |
| | 268, 269, 270, 271, 272, 273, 274, 275, 276, 277, 278, 279, 280, |
| | 281, 282, 283, 284, 285, 286, 287, 288, 289, 290, 291, 292, 293, |
| | 294, 295, 296, 297, 298, 299, 300, 301, 302, 303, 304, 305, 306, |
| | 307, 308, 309, 310, 311, 312, 313, 314, 315, 316, 317, 318, 319, |
| | 320, 321, 322, 323, 324, 325, 326, 327, 328, 329, 330, 331, 332, |
| | 333, 334, 335, 336, 337, 338, 339, 340, 341, 342, 343, 344, 345, |
| | 346, 347, 348, 349, 350, 351, 352, 353, 354, 355, 356, 357, 358, |
| | 359, 360, 361, 362, 363, 364, 365, 366, 367, 368, 369, 370, 371, |
| | 372, 373, 374, 375, 376, 377, 378, 379, 380, 381, 382, 383, 384, |
| | 385, 386, 387, 388, 389 and 390. |

Methods Using the Cre/lox System

In each method described, any of the Cre/lox combinations detailed herein, such as the combinations delineated in either Table J or K, may be utilized. The Cre/lox systems of the invention may be utilized in several applications, including for conditional mutagenesis and gene expression, gene replacement and deletion, and chromosome engineering.

It is contemplated that the mutant lox sites of the invention may be introduced into a nucleic acid in a number of different orientations in order to achieve a desired recombination result for any given application. Since a mutant lox site is an asymmetrical nucleotide sequence, two mutant lox sites on the same DNA molecule can have the same or opposite orientation with respect to each other. In one embodiment, recombination between mutant lox sites in the same orientation results in a deletion of the DNA segment located between the two mutant lox sites and a connection between the resulting ends of the original DNA molecule. The deleted DNA segment forms a circular molecule of DNA. The original DNA molecule and the resulting circular molecule each contain a single lox site. Alternatively, recombination between two mutant lox sites in opposite orientations on the same DNA molecule result in an inversion of the nucleotide sequence of the DNA segment located between the two mutant lox sites. In addition, reciprocal exchange of DNA segments proximate to mutant lox sites located on two different DNA molecules can occur.

One embodiment encompasses use of the Cre/lox system of the invention in a method for producing a site-specific recombination in a nucleotide sequence having a target DNA segment. In this method, a first and second mutant lox site of the invention is introduced into the nucleotide sequence such that the lox sites flank the target DNA segment. The nucleotide sequence may be either in vitro, such as a plasmid in a reaction tube, or it may be in vivo, such as in a cell. The target DNA segment can be a gene or a number of other sequences of deoxyribonucleotides of homologous, heterologous or synthetic origin. In an exemplary embodiment, the target DNA segment is a gene for a structural protein, an enzyme, a regulatory molecule; or a DNA sequence that influences gene expression in the cell such as a regulatory nucleotide sequence, a promoter, or a polyadenylation nucleotide sequence. In one embodiment, the first and second mutant lox sites have formula (I). In still another embodiment, the first and second mutant lox sites have formula (II). In a more typical embodiment, the first and second mutant lox sites have formula (III). In a further embodiment, the first and second lox sites have formula (IV). In yet another embodiment, the first and second lox sites have formula (V). In still another embodiment, the first and second lox sites have formula (VI). In an additional embodiment, the first and second lox sites have formula (VII). The nucleotide sequence comprising the target DNA segment flanked by the first and second mutant lox sites are then contacted with a Cre mutant polypeptide of the invention. The contact may take place either in vitro or in vivo. In a typical embodiment, the Cre mutant polypeptide will have any of SEQ ID NO. 2, 3, 4, or 5. A combination of any of the Cre mutant and lox mutant polypeptides of the invention may be utilized, such as the combinations described in Tables J and K. In a preferred embodiment, the Cre mutant polypeptide will be contacted with the lox sites as a Cre nucleotide sequence operably linked to an inducible regulatory sequence, such as any of the inducible promoters described above or otherwise generally known in the art, so that its expression can be triggered at a desired time. Alternatively, the Cre polypeptide can be contacted with the lox sites according to the methods described herein or generally known in the art. In one alternative of this embodiment, the first and second mutant lox sites have the same orientation, and contact with the Cre mutant produces a deletion of the target DNA segment. Alternatively, in another embodiment the first and second mutant lox sites have opposite orientation, and contact with the Cre mutant produces an inversion of the nucleotide sequence of the target DNA segment. In still another alternative of this embodiment, the first and second lox sites are introduced into two different nucleotide sequences and contact with the Cre mutant produces a reciprocal exchange of nucleotide sequence proximate to the lox sites.

Yet another preferred embodiment encompasses use of the Cre/lox system of the invention in a method comprising a means to selectively produce site-specific recombination in a number of different nucleotide sequences. For example, the method may comprise producing site-specific recombination at two, three, four, or even five or more different nucleotide sequences or at one or more sites within the same nucleotide sequence. The nucleotide sequences may be either in vitro, such as in a test tube, or it may be in vivo, such as the same cell or in a combination of different cells. By way of non-limiting example, when the method has two nucleotide sequences it typically will employ two Cre polypeptides that recognize lox site having different sequences. One Cre polypeptide employed recognizes wild-type lox sites, but not mutant lox sites having an extended spacer region. The other Cre polypeptide utilized is a Cre mutant polypeptide of the invention that recognizes mutant lox sites additional nucleotides in the spacer region. Advantageously, because the two Cre polypeptides catalyze site-specific recombination at different lox sites, the method provides a means to selectively catalyze site-specific recombination at the two target DNA segments either simultaneously or at different times. A method for producing site-specific recombination at two target DNA segments is described in detail below.

Accordingly, in one alternative of this embodiment site-specific recombination is selectively performed at a first and a second nucleotide sequence. The method employs four lox sites and two Cre polypeptides. In this embodiment, a first and second mutant lox site is introduced into the first nucleotide sequence such that the lox sites flank a first target DNA segment. The first and second mutant lox sites each have additional nucleotides in the spacer region according to any of the embodiments detailed above for mutant lox sites. Preferably, the first and second mutant lox sites selected will comprise the same nucleotide sequence. In one embodiment, the first and second mutant lox sites have formula (I). In still another embodiment, the first and second mutant lox sites have formula (II). In a more typical embodiment, the first and second mutant lox sites have formula (III). In a further embodiment, the first and second lox sites have formula (IV). In yet another embodiment, the first and second lox sites have formula (V). In still another embodiment, the first and second lox sites have formula (VI). In an additional embodiment, the first and second lox sites have formula (VII). The method also encompasses introducing a third and fourth lox site into a second nucleotide sequence such that the lox sites flank a second target DNA segment. The third and fourth lox sites comprise a wild-type lox site such as any of loxP, loxB, loxL, or loxR. In a typical embodiment, the third and fourth lox sites comprise wild-type loxP. Depending upon the embodiment, the third and fourth lox site may be introduced into either the same nucleotide sequence as the first and second mutant lox sites or into different nucleotide sequence. The method additionally comprises contacting the lox sites (i.e., either mutant or wild type) with an appropriate Cre polypeptide. The Cre polypeptide typically will be contacted with the nucleotide sequence comprising the lox sites as a Cre nucleotide sequence operably linked to an inducible regulatory sequence, such as any of the inducible promoters described above or otherwise generally known in the art, so that its expression can be triggered at a desired time. Alternatively, the Cre polypeptide can be contacted with the nucleotide sequence comprising the lox sites according to the methods described herein or generally known in the art. One of the Cre polypeptides will be a Cre mutant polypeptide of the invention that can catalyze site-specific recombination at a lox site having a spatially extended spacer region. The Cre mutant polypeptide and mutant lox site may be a combination of any described herein, such as the combination detailed in either Table J or K. The method also encompasses contacting the third and fourth lox sites with a second Cre polypeptide. An appropriate Cre polypeptide, in this case, will be able to catalyze site specific recombination at wild-type lox sites, but not at lox sites having additional nucleotides in the spacer region. Again, the Cre polypeptide may be either be contacted with the nucleotide sequence comprising lox sites as a nucleotide sequence operably linked to an inducible regulatory sequence or the polypeptide may be contacted with the nucleotide sequence comprising the lox sites. In one embodiment, when the third and fourth lox sites are wild-type loxP sites, the Cre polypeptide has SEQ ID NO. 1. Depending upon the particular embodiment, the first and second mutant lox sites may be contacted with the Cre mutant polypeptide either before, simultaneously, or after the third and fourth wild-type lox sites are contacted with the wild-type Cre polypeptide. In one alternative of this embodiment, the pairs of lox sites (i.e., first and second mutant lox site and third and fourth wild-type lox sites) have the same orientation, and contact with the particular Cre polypeptide produces a deletion of the target DNA segment. Alternatively, in another embodiment the pairs of lox sites have opposite orientation, and contact with the particular Cre polypeptide produces an inversion of the nucleotide sequence of the target DNA segment. In still another alternative of this embodiment, the pairs of lox sites are each introduced into two different nucleotide sequences and contact with the particular Cre polypeptide produces a reciprocal exchange of nucleotide sequence proximate to the lox sites. In an additional embodiment, one pair of lox sites is introduced in opposite orientation and the other pair of lox sites is introduced in the same orientation. In still another embodiment, one pair of lox sites is introduced in opposite orientation and the other pair of lox sites is introduced on two separate nucleotide sequences. In yet another embodiment, one pair of lox sites is introduced in the same orientation and the other pair of lox sites is introduced on two separate nucleotide sequences.

In one exemplary application, the methods of the invention will be utilized for conditional activation of transgene expression such as to knock-in a target DNA segment, such as a gene, by use of a site-specific recombination reaction that is catalyzed by a Cre mutant polypeptide of the invention. One preferred use for the knock-in embodiment, is for introduction of a target DNA segment into a chromosome or into a transgenic animal, such as a mouse. In this method, a first nucleotide construct comprising a nucleotide sequence encoding a Cre mutant polypeptide operably linked to a promoter is used to site-specifically recombine a second nucleotide construct comprising two mutant lox sites, a target DNA segment to be knocked-in, and a promoter. In a typical embodiment, the promoter employed to express the Cre mutant polypeptide will be an inducible promoter so that the target DNA segment can be knocked-in by the Cre mutant at a time and location controlled manner. In a typical arrangement of the second nucleotide construct, the promoter is arranged upstream of a first mutant lox site and the second mutant lox site is downstream of the first mutant lox site, with an intervening nucleotide sequence disposed between the first and second mutant lox sites. The promoter is preferably arranged so as to induce the expression of the target DNA segment to be knocked-in. An exemplary second nucleotide construct has the following arrangement:

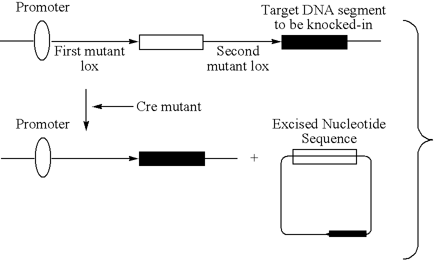

When the Cre polypeptide is contacted with the mutant lox sites, it binds to the sites and removes the intervening nucleotide sequence disposed between the first and second mutant lox sites (see diagram above). After the Cre polypeptide has excised the intervening nucleotide sequence, the first mutant lox site is left behind and the target DNA segment is operably linked to the promoter.

Alternatively, in yet another exemplary application, the methods of the invention will be utilized to knock-out a target DNA segment, such as a gene, by use of a site-specific recombination reaction that is catalyzed by a Cre mutant polypeptide of the invention. The method is typically employed to terminate expression of a gene. In many respects, the knocking-out method is performed in a substantially similar manner as the knocking-in method except the position of the promoter sequence in relation to the target DNA segment in the second nucleotide construct is different. Because the knocking-out method is employed primarily as a means to terminate gene expression, it is satisfactory if either the target DNA segment or the promoter sequence are knocked-out, either in whole or in part, from the second nucleotide construct. Suitable examples of arrangements for the first and second mutant lox sites, the promoter sequence, and the target DNA segment within the second nucleotide construct are included in examples (a), (b) or (c):
(a)—promoter-first mutant lox site-target DNA segment-second mutant lox site—
(b)—first mutant lox site—promoter—target DNA segment—second mutant lox site—
(c)—first mutant lox site—promoter—second mutant lox site—target DNA segment

The knock-out method also encompasses a first nucleotide construct comprising a nucleotide sequence encoding a Cre mutant polypeptide operably linked to a promoter. In a typical embodiment, the promoter employed to express the Cre mutant polypeptide will be an inducible promoter so that the target DNA segment can be knocked-out by the Cre mutant at a time and location controlled manner. When the Cre polypeptide is contacted with the mutant lox sites, it binds to the sites and removes the intervening nucleotide sequence disposed between the first and second mutant lox sites. Depending upon the arrangement of the second nucleotide construct, the intervening nucleotide sequence may include all or a part of the promoter or the target DNA segment, or both. This nucleotide sequence excision results in a loss of target DNA segment function, or loss of promoter function or both. A schematic showing a typical embodiment of knock-out of a target DNA segment is as follows:

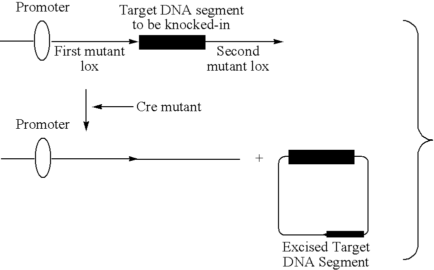

The knock-in and knock-out methods described above may be utilized to introduce or excise a target DNA segment in a variety of in vivo or in vitro applications and in several organisms. By way of non-limiting example, the methods may be employed as a tool for conditional mutagenesis and gene expression, gene replacement and deletion, and chromosome engineering.

In one exemplary embodiment, the recombination methods detailed herein are employed to produce a variety of transgenic non-human organisms. The transgenic organisms may be produced by the methods described herein or methods that are generally known in the art, such as by using homologous recombination in embryonic stem cells (See, e.g., U.S. Pat. No. 5,175,383 and U.S. Pat. No. 5,767,337.). For example when utilizing a knock-out method, mouse embryonic stem (ES) cells, such as the mouse 129/SvJ cell line, are derived from the early mouse embryo and grown in culture. Homologous recombination takes place using the Cre-lox system of the invention to knock-out a gene of interest in a tissue- or developmental stage-specific manner, as described above or as known in the art (Marth, J. D. (1996) Clin. Invest. 97:1999-2002; Wagner, K. U. et al. (1997) Nucleic Acids Res. 25:4323-4330). Transformed ES cells are identified and introduced into mouse cell blastocysts such as those from the C57BL/6 mouse strain. The blastocysts are surgically transferred to pseudopregnant dams, and the resulting chimeric progeny are genotyped and bred to produce heterozygous or homozygous strains. Alternatively, when utilizing a knock-in method, polynucleotides encoding a target DNA segment can be used to create transgenic animals (mice or rats). Typically, a region of a polynucleotide encoding a target DNA segment is injected into animal embryonic stem cells, and the injected sequence integrates into the animal cell genome. Transformed cells are injected into blastulae, and the blastulae are implanted as described above.

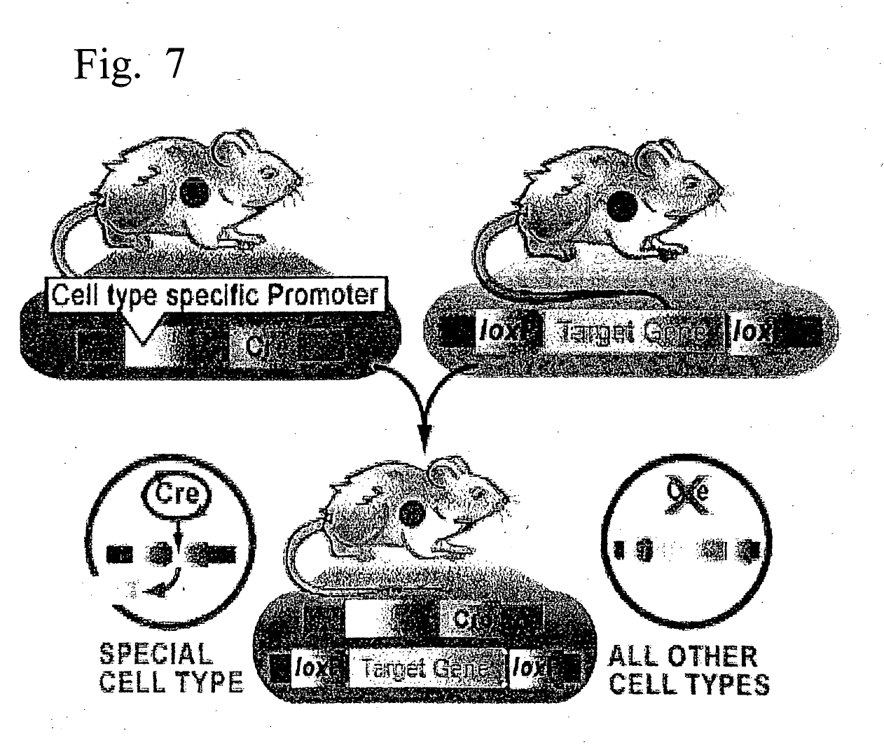

In one non limiting example of a transgenic animal that may be produced in the practice of the invention, a knock-out mouse that no longer has a target gene in a particular cell type can be produced. Referring to FIG. 7, a transgenic mouse containing a target gene flanked by mutant lox sites is mated with a transgenic mouse that expresses a Cre mutant gene in only one cell type. The mouse resulting from this breeding will have both the Cre mutant gene and the mutant lox-flanked gene. In cells of the mouse that don't express the Cre mutant polypeptide, the target gene will function normally. Alternatively, in a cell where the Cre mutant is expressed, the target gene will be deleted. In a preferred alternative of this embodiment, the target gene will be conditionally knocked-out. A conditional knock-out mouse can be produced if the Cre mutant gene is operably linked to an inducible or tissue specific promoter. When conditions needed for promoter function are provided, Cre mutant polypeptide is expressed and the target gene is knocked out. Alternatively, if conditions needed for promoter function are not provided, Cre mutant polypeptide is not expressed and the target gene is not knocked-out.

Introduction of Cre Mutant Sequences and Mutant lox Sequences

Irrespective of the particular use of the Cre/lox system of the invention, a number of methods are suitable for introducing mutant lox site nucleotide sequences and Cre mutant nucleotide sequences into a nucleic acid molecule or a target cell. The method selected for such introduction can and will vary depending upon the particular sequence and target cell. Generally speaking, the cell may be an in vivo or in vitro cell. For example, the nucleotide sequences can be expressed by a recombinant cell, such as a bacterial cell, a cultured eukaryotic cell, or a cell disposed in a living organism, including a non-human transgenic organism, such as a transgenic animal. By way of non-limiting example, cultured cells available for use include Hela cells, HEK 293 cells and U937 cells, as well as other cells used to express proteins.

In one exemplary embodiment of the invention, a vector, such as a vector detailed above, can be employed to introduce a suitable mutant lox site or Cre mutant polynucleotide into a host cell. Typically, in this aspect of the invention, the polynucleotide is incorporated into an expression vector, which subsequently is utilized to transfect a target cell. Depending upon the embodiment, the cell may be a cultured cell or a cell disposed within a living organism. Irrespective of the embodiment, the vector binds to the target cell membrane, and the subject nucleotide sequence is internalized into the cell. The vector comprising the nucleotide sequence (i.e., mutant lox site or Cre mutant) may be either integrated into the target cell's nucleic acid sequence or may be a plasmid. Irrespective of its form, the vector employed results in Cre mutant polypeptide expression and insertion of mutant lox sites at a desired location.

In one embodiment, the transfer vector is a retrovirus. Retroviruses can package up to 5 Kb of exogenous nucleic acid material, and can efficiently infect dividing cells via a specific receptor, wherein the exogenous genetic information is integrated into the target cell genome. In the host cell cytoplasm, the reverse transcriptase enzyme carried by the vector converts the RNA into proviral DNA, which is then integrated into the target cell genome, thereby expressing the transgene product.

In another alternative embodiment, the transfer vector is an adenovirus. In general, adenoviruses are large, double-stranded DNA viruses which contain a 36 Kb genome that consists of genes encoding early regulatory proteins and a late structural protein gene. Adenoviruses, advantageously, can be grown in high titers of purified recombinant virus (up to 10¹² infectious particles/ml), incorporate large amounts of exogenous genetic information, and can broadly infect a wide range of differentiated non-dividing cells in vivo.

In yet another alternative embodiment, the transfer vector is an adeno-associated virus (AAV). AAV is a human parvovirus that is a small, single-stranded DNA virus that can infect both dividing and non-dividing cells. AAV is relatively non-toxic and non-immunogenic and results in long-lasting expression. The packaging capacity of recombinant AAV is 4.9 kb. Successful AAV-mediated gene transfer into brain, muscle, heart, liver, and lung tissue has been reported.

Exemplary transfer vectors for transfer into eukaryotic cells include MSCV, Harvey murine sarcoma virus, pFastBac, pFastBac HT, pFastBac DUAL, pSFV, pTet-Splice, pEUK-Cl, pPUR, pMAM, pMAMneo, pBI101, pBI121, pDR2, pCMVEBNA, YACneo, pSVK3, pSVL, pMSG, pCH110, pKK232-8, p3′SS, pBlueBacIII, pCDM8, pcDNA1, pZeoSV, pcDNA3, pREP4, pET21b, pCEP4, and pEBVHis vectors.

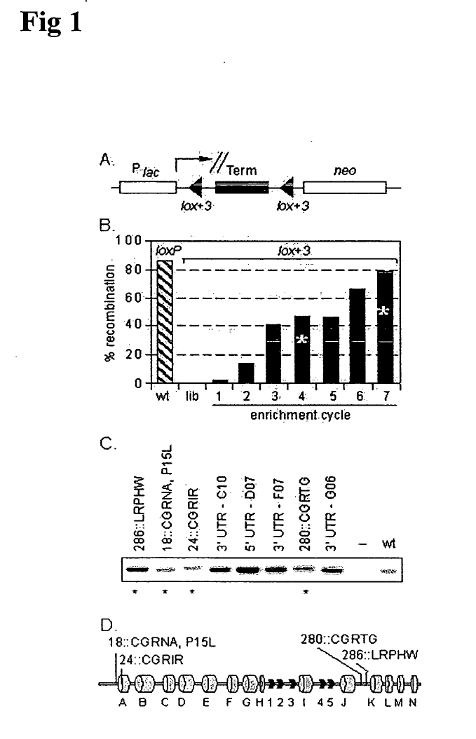

In one embodiment and by way of non limiting example, the vector will be the Ap^{R} reporter plasmid depicted in FIG. 1 and further described in the examples. Briefly, the Ap^{R} plasmid carries two directly repeated lox⁺³ sites flanking a rrn T1T2 transcription terminator (Term) interposed between the lac promoter and neo. Cre-mediated excision at the lox sites allows neo expression to give kanamycin resistance.

The transfected cells include isolated in vitro population of cells. In vivo, the vector can be delivered to selected cells, whereby the carrier for the vector is attracted to the selected cell population.

Activation of the gene in a transfected cell can be caused by an external stress factor. For example, the transfected cells can be contacted with an etoposide or a proteosome inhibitor. In the alternative, an activator can be included in the vector in accordance with the methods detailed above.

In another alternative embodiment, the mutant lox site or Cre mutant nucleotide sequences can be introduced into a target cell by mechanical, electrical or chemical procedures. Mechanical methods include microinjection, pressure, and particle bombardment. Electrical methods include electroporation. Chemical methods include liposomes, DEAE-dextran, calcium phosphate, artificial lipids, proteins, dendrimers, or other polymers, including controlled-release polymers. In one aspect of this embodiment, accordingly, a mechanical method is employed to introduce the subject nucleotide sequences into the target cell. One such method is hydrodynamic force and other external pressure-mediated DNA transfection methods. Alternatively, ultrasonic nebulization can be utilized for DNA-lipid complex delivery. In other suitable embodiments, particle bombardment, also known as biolistical particle delivery, can be utilized to introduce DNA into several cells simultaneously. In still another alternative mechanical method, DNA-coated microparticles (e.g., gold, tungsten) are accelerated to high velocity to penetrate cell membranes or cell walls. This procedure is used predominantly in vitro for adherent cell culture transfection.

In a further aspect of this embodiment, an electrical method is employed to introduce subject nucleotide sequences into the target cell. In one alternative of this embodiment, electroporation is employed. Electroporation uses high-voltage electrical impulses to transiently permeabilize cell membranes, and thereby, permits cellular uptake of macromolecules, such as nucleic acid and polypeptide sequences.

In an additional aspect of this embodiment, a chemical method is employed to introduce a selected nucleotide sequences into the target cell. Chemical methods, using uptake-enhancing chemicals, are highly effective for delivering nucleic acids across cell membranes. For example, nucleotide sequences are typically negatively charged molecules. DEAE-dextran and calcium phosphate, which are positively charged molecules, interact with nucleotide sequences to form DEAE-dextran-DNA and calcium phosphate-DNA complexes, respectively. These complexes are subsequently internalized into the target cell by endocytosis.

In another alternative embodiment, the chemical enhancer is lipofectin-DNA. This complex comprises an artificial lipid-based DNA delivery system. In this embodiment, liposomes (either cationic, anionic, or neutral) are complexed with DNA. The liposomes can be used to enclose a subject nucleic acid for delivery to target cells, in part, because of increased transfection efficiency. In yet another alternative chemical embodiment, protein-based methods for DNA introduction may also utilized. The cationic peptide poly-L-lysine (PLL) can condense DNA for more efficient uptake by cells. Protamine sulfate, polyamidoamine dendrimers, synthetic polymers, and pyridinium surfactants may also be utilized.

In still a further chemical embodiment for nucleotide introduction, biocompatible controlled-release polymers may be employed. Biodegradable poly (D,L-lactide-co-glycolide) microparticles and PLGA microspheres have been used for long-term controlled release of DNA molecules to cells. In a further embodiment, the subject nucleotide sequences may also be encapsulated into poly(ethylene-co-vinyl acetate) matrices, resulting in long term controlled, predictable release for several months.

Similarly, as for the introduction of Cre mutant nucleotide sequences, the Cre mutant polypeptide can also be introduced into target cells by any of the mechanical, electrical or chemical means detailed above. Mechanical methods include microinjection, pressure, and particle bombardment. Direct microinjection of Cre mutant polypeptide into cells in vitro occurs directly and efficiently. As with DNA-injected cells, once cells are modified in vitro, they can be transferred to the in vivo host environment. In particle bombardment, Cre mutant polypeptide-coated microparticles are physically hurled with force against cell membranes or cell walls to penetrate cells in vitro. Electroporation, particularly at low voltage, and high frequency electrical impulses, is suitable for introduction of Cre mutant polypeptides with in vitro or in vivo. Moreover, any of the chemical means detailed above may also be employed.

The invention also encompasses nucleic acid constructs, cells and organisms having a Cre mutant (i.e., nucleotide or polypeptide), mutant lox site, or both a Cre mutant and mutant lox site. The Cre mutant, lox site, or Cre/lox combination may be any such sequence described herein, such as the combinations specifically detailed in Tables J and K.

Production of Antibodies Specific for Cre Mutant Polypeptides

Yet a further aspect of the invention encompasses the use of Cre mutant polypeptides or proteins to produce antibodies. The antibodies may be employed in in vitro and in vivo assays or to purify a Cre mutant polypeptide. Antibodies to any of the polypeptides suitable for use in the invention may be generated using methods that are well known in the art. Such antibodies may include, but are not limited to, polyclonal, monoclonal, chimeric, and single chain antibodies, Fab fragments, and fragments produced by a Fab expression library.

For the production of antibodies, various hosts including goats, rabbits, rats, mice, humans, and others may be immunized by injection with a subject polypeptide that has immunogenic properties. Depending on the host species, various adjuvants may be used to increase immunological response. Such adjuvants include, but are not limited to, Freund's, mineral gels such as aluminum hydroxide, and surface-active substances such as lysolecithin, pluronic polyols, polyanions, peptides, oil emulsions, KLH, and dinitrophenol. Among adjuvants used in humans, BCG (bacilli Calmette-Guerin) and Corynebacterium parvum are especially preferable.

It is preferred that the oligopeptides, peptides, or fragments used to induce antibodies to a selected polypeptide have an amino acid sequence consisting of at least about 5 amino acids, and generally will consist of at least about 10 amino acids. It is also preferable that these oligopeptides, peptides, or fragments are identical to a portion of the amino acid sequence of the natural protein. Short stretches of the selected polypeptide's amino acid may be fused with those of another protein, such as KLH, and antibodies to the chimeric molecule may be produced.

Monoclonal antibodies to a polypeptide may be prepared using a technique that provides for the production of antibody molecules by continuous cell lines in culture. These include, but are not limited to, the hybridoma technique, the human B-cell hybridoma technique, and the EBV-hybridoma technique. (See, e.g., Kohler, G. et al. (1975) Nature 256:495-497; Kozbor, D. et al. (1985) J. Immunol. Methods 81:3142; Cote, R. J. et al. (1983) Proc. Natl. Acad. Sci. USA 80:2026-2030; and Cole, S. P. et al. (1984) Mol. Cell Biol. 62:109-120.)

In addition, techniques developed for the production of “chimeric antibodies,” such as the splicing of mouse antibody genes to human antibody genes to obtain a molecule with appropriate antigen specificity and biological activity can be used. (See, e.g., Morrison, S. L. et al. (1984) Proc. Natl. Acad. Sci. USA 81:6851-6855; Neuberger, M. S. et al. (1984) Nature 312:604-608; and Takeda, S. et al. (1985) Nature 314:452-45). Alternatively, techniques described for the production of single chain antibodies may be adapted, using methods known in the art, to produce Cre mutant polypeptide-specific single chain antibodies. Antibodies with related specificity, but of distinct idiotypic composition, may be generated by chain shuffling from random combinatorial immunoglobulin libraries. (See, e.g., Burton, D. R. (1991) Proc. Natl. Acad. Sci. USA 88:10134-10137.)

Antibodies may also be produced by inducing in vivo production in the lymphocyte population or by screening immunoglobulin libraries or panels of highly specific binding reagents as disclosed in the literature. (See, e.g., Orlandi, R. et al. (1989) Proc. Natl. Acad. Sci. USA 86:3833-3837; Winter, G. et al. (1991) Nature 349:293-299.)

Antibody fragments that contain specific binding sites for Cre mutant polypeptides may also be generated. For example, such fragments include, but are not limited to, F(ab′)₂ fragments produced by pepsin digestion of the antibody molecule and Fab fragments generated by reducing the disulfide bridges of the F(ab′)₂ fragments. Alternatively, Fab expression libraries may be constructed to allow rapid and easy identification of monoclonal Fab fragments with the desired specificity. (See, e.g., Huse, W. D. et al. (1989) Science 246:1275-1281.)

Various immunoassays may be used for screening to identify antibodies having the desired specificity. Numerous protocols for competitive binding or immunoradiometric assays using either polyclonal or monoclonal antibodies with established specificities are well known in the art. Such immunoassays typically involve the measurement of complex formation between the polypeptide and its specific antibody. A two-site, monoclonal-based immunoassay utilizing monoclonal antibodies reactive to two non-interfering polypeptide epitopes is generally used, but a competitive binding assay may also be employed.

Various methods such as Scatchard analysis in conjunction with radioimmunoassay techniques may be used to assess the affinity of antibodies for the subject polypeptide. Affinity is expressed as an association constant, Kₐ, which is defined as the molar concentration of polypeptide-antibody complex divided by the molar concentrations of free antigen and free antibody under equilibrium conditions. The Kₐ is determined for a preparation of polyclonal antibodies, which are heterogeneous in their affinities for multiple polypeptide epitopes, represents the average affinity, or avidity, of the antibodies for the particular polypeptides. The Kₐ is determined for a preparation of monoclonal antibodies, which are monospecific for a particular polypeptide epitope, represents a true measure of affinity. High-affinity antibody preparations with Kₐ ranging from about 10⁹ to 10¹² L/mole are preferred for use in immunoassays in which the polypeptide-antibody complex must withstand rigorous manipulations. Low-affinity antibody preparations with Kₐ ranging from about 10⁶ to 10⁷ L/mole are preferred for use in immunopurification and similar procedures that ultimately require dissociation of polypeptides, preferably in active form, from the antibody (Catty, D. (1988) Antibodies, Volume I: A Practical Approach, IRL Press, Washington D.C.; Liddell, J. E. and A. Cryer (1991) A Practical Guide to Monoclonal Antibodies, John Wiley & Sons, New York N.Y.).

The titer and avidity of polyclonal antibody preparations may be further evaluated to determine the quality and suitability of such preparation for certain downstream applications. For example, a polyclonal antibody preparation containing at least 1-2 mg specific antibody/ml, preferably 5-10 mg specific antibody/ml, is generally employed in procedures requiring precipitation of a subject polypeptide-antibody complex. Procedures for evaluating antibody specificity, titer, and avidity, and guidelines for antibody quality and usage in various applications, are generally available. (See, e.g., Catty, supra, and Coligan et al. supra.) Generally speaking, the antibodies of the invention may be utilized in a variety of applications such as for protein purification or for therapeutic uses. Alternatively, the antibodies are also used as tools to mark the presence of the Cre mutant protein. The marker antibodies include a marker, such as a fluorescent marker, and will bind to the wt Cre protein.

Kits

A further aspect of the invention encompasses kits that employ the Cre/lox system of the invention.

In one embodiment, the kit is for producing site-specific recombination of a target DNA segment. Typically, a kit in this embodiment will include a purified mutant Cre polypeptide that can catalyze site specific recombination at a lox site having from one to three additional nucleotides in the spacer region. By way of example, the Cre mutant polypeptide may be any of SEQ ID NO. 2, 3, 4, or 5. The kit also comprises two isolated mutant lox nucleotide sequences having from one to three additional nucleotides in the spacer region. The lox sites may have any of formula (I), (II), (III), (IV), (V), (VI) or (VII). Suitable examples of Cre/lox combination are detailed in Tables J and K. The kit will also include instructions for producing site-specific recombination of a target DNA segment.

In yet another embodiment, the kit is for producing selective site-specific recombination of two or more different target DNA segments. The kit comprises two different purified Cre polypeptides and two pairs of different isolated lox sites. The first Cre polypeptide is a Cre mutant polypeptide of the invention that can catalyze site-specific recombination of mutant lox sites having from one to three additional nucleotides in the spacer region. Non limiting examples of suitable Cre mutant polypeptides include SEQ ID NO. 2, 3, 4, or 5. The second Cre polypeptide is a Cre polypeptide that can catalyze site specific recombination at a wild-type lox site, but not lox sites having three additional nucleotides in the spacer region. SEQ ID NO. 1 represents an example of a suitable Cre polypeptide. The first lox site included in the kit is a mutant lox site of the invention having from one to three additional nucleotides in the spacer region. Suitable mutant lox sites having three additional nucleotides in the spacer region include those lox sites having any of formula (I), (II), (III), (IV), (V), (VI) or (VII). Suitable examples of Cre/lox combination are detailed in Tables J and K. The second lox site included in the kit is typically a wild-type lox site that is recognized by the Cre polypeptide provided in the kit. For example, if the Cre polypeptide has SEQ ID NO. 1, then the lox site provided will be a wild-type loxP site. The kit will also include instructions for producing selective site-specific recombination in the target DNA segments.

All publications, patents, patent applications and other references cited in this application are herein incorporated by reference in their entirety as if each individual publication, patent, patent application or other reference were specifically and individually indicated to be incorporated by reference.

### Definitions

Cell as used herein refers to either a prokaryotic cell or an eukaryotic cell. Examples of such cells include bacterial cells, yeast cells, mammalian cells, plant cells, insect cells or fungal cells.

Conservative amino acid substitutions are those substitutions that do not abolish the ability of a subject polypeptide to participate in the biological functions as described herein. Typically, a conservative substitution will involve a replacement of one amino acid residue with a different residue having similar biochemical characteristics such as size, charge, and polarity versus non polarity. A skilled artisan can readily determine such conservative amino acid substitutions.

DNA segment refers to a linear fragment of single- or double-stranded deoxyribonucleic acid (DNA), which can be derived from any source.

Efficiency refers to the amount of substrate converted to product in any given reaction. The term is used herein to describe the site-specific recombination reaction at a particular lox site or mutant lox site catalyzed by a Cre mutant polypeptide of the invention or by a wild-type Cre polypeptide. Efficiency is measured according to either the in vitro or in vivo recombination assays detailed in the examples.

The term expression as used herein is intended to mean the synthesis of gene product from a gene coding for the sequence of the gene product. The gene product can be RNA or a protein.

A gene is a hereditary unit that has one or more specific effects upon the phenotype of the organism that can mutate to various allelic forms.

Higher Efficiency is used herein as a means to compare the relative efficiencies of Cre mutant polypeptides of the invention to wild-type Cre polypeptide when catalyzing site-specific recombination at a particular lox site. A Cre mutant polypeptide has a higher efficiency compared to a wild-type Cre polypeptide if the mutant can covert a greater amount of a particular lox site a to a particular product by site-specific recombination. A suitable Cre mutant of the invention generally has at least about a 5-fold to about 1000-fold higher efficiency compared to wild-type Cre for catalyzing site specific recombination at a lox site having an extended spacer region, as detailed herein. More typically, a suitable Cre mutant of the invention will have at least about a 25-fold to about a 1000-fold higher efficiency compared to wild-type Cre for catalyzing site specific recombination at a lox site having an extended spacer region, as detailed herein. In one preferred embodiment, the Cre mutant will have greater than a 50-fold higher efficiency compared to wild-type Cre for catalyzing site specific recombination at a lox site having an extended spacer region, as detailed herein. In a particularly preferred embodiment, the Cre mutant will have greater than a 500-fold higher efficiency compared to wild-type Cre for catalyzing site specific recombination at a lox site having an extended spacer region, as detailed herein.

Homology describes the degree of similarity in nucleotide or protein sequences between individuals of the same species or among different species. As the term is employed herein, such as when referring to the homology between either two proteins or two nucleotide sequences, homology refers to molecules having substantially the same function, but differing in sequence. Most typically, the two homologous molecules will share substantially the same sequence, particularly in conserved regions, and will have sequence differences in regions of the sequence that does not impact function.

A host organism is an organism that receives a foreign biological molecule, including an antibody or genetic construct, such as a vector containing a gene. The organism may be either a prokaryote or an eukaryote. For example, the organism may be a bacteria, a yeast, a mammal, a plant, an insect, or a fungus.

Knock-in, as used herein, is commonly understood to be the placement into the genome by homologous recombination of a transgene at a specific locus such that it is under the regulatory control of genetic elements endogenous to that locus. In a typical embodiment, a knock-in procedure will be used to substitute the transgene for an endogenous gene in the genome of the transgenic organism.

Knock-out, as used herein, is commonly understood to be the placement into the genome by homologous recombination of a transgene at a specific locus such that placement of the transgene results in the ablation of an endogenous gene at the specific locus.

The loop between the J and K helices, as used herein, generally refers to a region from about residue 270 to about residue 290 of SEQ ID NO. 1.

As used herein the expression lox site means a nucleotide sequence at which the gene product of the cre gene, referred to herein as Cre, can catalyze a site-specific recombination. The loxP site is a 34 base pair nucleotide sequence that can be isolated from bacteriophage P1 by methods known in the art. One method for isolating a loxP site from bacteriophage P1 is disclosed by Hoess et al., Proc. Natl. Acad. Sci. USA, 79: 3398 (1982). The loxP site consists of two 13 base pair inverted repeats separated by an 8 base pair spacer region Other suitable lox sites include loxB, loxL and loxR sites which are nucleotide sequences isolated from E. coli. These sequences are disclosed and described by Hoess et al., Proc. Natl. Acad. Sci. USA, 79: 3398 (1982). Lox sites can also be produced by a variety of synthetic techniques that are known in the art. For example, synthetic techniques for producing lox sites are disclosed by Ito et al., Nuc. Acid Res., 10: 1755 (1982) and Ogilvie et al., Science, 214: 270 (1981).

Mutation is defined as a phenotypic variant resulting from a changed or new gene.

Mutant is an organism bearing a mutant gene that expresses itself in the phenotype of the organism. Mutants include both changes to a nucleic acid sequence, as well as elimination of a sequence or a part of a sequence. In addition polypeptides can be expressed from the mutants.

The N-terminus of helix A, as used herein, generally refers to a region from residue 1 to about residue 30 of SEQ ID NO. 1.

A nucleic acid is a nucleotide polymer better known as one of the monomeric units from which DNA or RNA polymers are constructed, it consists of a purine or pyrimidine base, a pentose, and a phosphoric acid group.

Peptide is defined as a compound formed of two or more amino acids, with an amino acid defined according to standard definitions, such as is found in the book “A Dictionary of Genetics” by King and Stansfield.

Plasmids are double-stranded, closed DNA molecules ranging in size from 1 to 200 kilo-bases. Plasmids are incorporated into vectors for transfecting a host with a nucleic acid molecule.

A polypeptide is a polymer made up of less than 350 amino acids.

Protein is defined as a molecule composed of one or more polypeptide chains, each composed of a linear chain of amino acids covalently linked by peptide bonds. Most proteins have a mass between 10 and 100 kilodaltons. A protein is often symbolized by its mass in kDa.

Polyadenylation nucleotide sequence or polyadenylation nucleotide region refers to a nucleotide sequence usually located 3′ to a coding region which controls the addition of polyadenylic acid to the RNA transcribed from the coding region in conjunction with the gene expression apparatus of the cell.

As used herein, the term promoter region refers to a sequence of DNA, usually upstream (5′) of the coding sequence, which controls the expression of the coding region by providing the recognition for RNA polymerase and/or other factors required for transcription to start at the correct site. A “promoter fragment” constitutes a DNA sequence consisting of the promoter region. A promoter region can include one or more regions that control the effectiveness of transcription initiation in response to physiological conditions, and a transcription initiation sequence.

Regulatory nucleotide sequence as used herein, refers to a nucleotide sequence located proximate to a coding region whose transcription is controlled by the regulatory nucleotide sequence in conjunction with the gene expression apparatus of the cell. Generally, the regulatory nucleotide sequence is located 5′ to the coding region. A promoter can include one or more regulatory nucleotide sequences.

As used herein, the expression site-specific recombination is intended to include the following three events: (1) deletion of a target DNA segment flanked by lox sites, (2) inversion of the nucleotide sequence of a target DNA segment flanked by lox sites, and (3) reciprocal exchange of DNA segments proximate to lox sites located on different DNA molecules. It is to be understood that this reciprocal exchange of DNA segments can result in an integration event.

Substrate as used herein is a site within a nucleic acid sequence recognized by a particular recombinase, wherein the recombinase catalyzes site specific recombination. For example, the substrate for a Cre mutant polypeptide is a lox⁺³ site and the substrate for wild-type Cre recombinase is a loxP site.

Target DNA segment as employed herein can be a gene or a number of other sequences of deoxyribonucleotides of homologous, heterologous or synthetic origin. In an exemplary embodiment, the target DNA segment is a gene for a structural protein, an enzyme, a regulatory molecule; or a DNA sequence that influences gene expression in the cell such as a regulatory nucleotide sequence, a promoter, or a polyadenylation nucleotide sequence.

A vector is a self-replication DNA molecule that transfers a DNA segment to a host cell.

Wild-type is the most frequently observed phenotype, or the one arbitrarily designated as “normal”. Often symbolized by “+” or “WT.”

As various changes could be made in the above compounds, products and methods without departing from the scope of the invention, it is intended that all matter contained in the above description and in the examples given below, shall be interpreted as illustrative and not in a limiting sense.

### EXAMPLES

Examples 1-4 below detail the ability of the Cre mutant polypeptides of the invention to catalyze site specific recombination at lox sites having three additional nucleotides in the spacer region. The examples also illustrate the inability of wild-type Cre polypeptide to catalyze site specific recombination at lox sites having three additional nucleotides in the spacer region.

In the examples below, where indicated, the following experimental procedures and reagents were employed:

Bacterial Strains and Plasmids:

Plasmids were constructed and propagated using E. coli DH5α (Invitrogen). Plasmids carrying two directly repeated wt or mutant lox sites flanking the rrnB T1T2 transcription terminator (“lox²” or “lox³” plasmids) were constructed as previously described (12) using synthetic oligonucleotides carrying the wt or mutant lox site (FIG. 1) flanked by XhoI and NheI restriction sites. The wt lox site used and all spacer length mutant sites carried a T to C variation at the second nucleotide from the outside end of one inverted repeat that does not affect Cre recombination (13). Selection plasmid derivatives of each of these lox² or lox³ plasmids were made by removing Ap^{R} and the ColE1 replication origin by digestion with BamHI-HindIII and replacing this region with the lac promoter from pUC19 and the Cm^{R} marker and replication origin from pACYC 184. Thus, pBS848, pBS849, pBS808 and pBS827 are pACYC-based Cm^{R} plasmids carrying the rrnB T1T2 terminator flanked by wt lox, lox⁺¹, lox⁺² or lox⁺³ sites, respectively, all inserted between the neo gene and a lac promoter. For all lox sites pACYC184-based inversion substrate plasmids were also constructed using a similar synthetic oligonucleotide-based method so that two identical lox sites were in inverted orientation to each other and separated by 381 bp. Inversion lox plasmids served as templates in the generation of substrates to monitor Cre-mediated synapsis, as described below. Insertion of the XbaI-HindIII cre fragment from pBAD33-cre (12) into pBAD18 (14) gave the Cre expression vector pBS809.

Construction of a Cre Library with Random Pentapeptide Insertions

Random pentapeptide insertions into cre were generated using the Mutation Generation System™ (Finnzymes, Finland) according to the manufacturer's instructions. Briefly, an artificial Mu transposon was randomly inserted in vitro into the 5657 bp Ap^{R} plasmid pBS809, a derivative of pBAD18 (15) in which cre is under the control of an arabinose-inducible promoter, and transformed into DH5α (Invitrogen) to yield 3×10⁵ independent colonies. Assuming random Mu transposition, this represents about 75-fold excess of insertion events into the ˜4000 non-essential bp of this plasmid. The 1067 bp HindIII-XbaI fragment containing Mu insertions and the coding sequence of Cre was subcloned into pBAD18 vector followed by deletion of the Mu transposon by NotI digest, religation and retransformation to yield the final pentapeptide insertion library. A minimum of 10⁵ independent cre plasmid colonies (100× coverage) was maintained throughout subsequent steps to ensure maximal representation of insertions within the 1067 bp HindIII-XbaI fragment containing 1029 bp of cre coding sequence. To confirm the randomness of the insertions we sequenced 184 independent transformants. Aside from several short regions without insertions, probably due to some low level bias in target selection by MuA transposase (16) or possible toxicity of the cre mutants to the host, the pattern of insertions appeared nearly random.

Selection Procedure

Insertion mutants of Cre that retain recombinase activity were selected by their ability to excise a lox⁺³-flanked transcription terminator (17) that prevents expression of a neo gene. Briefly, the expression library of Cre mutants was electroporated into DH5α [pBS848], where pBS848 is a pACYC-based Cm^{R} plasmid carrying the lox⁺³ rrnB T1T2 terminator cassette inserted between the neo gene and a lac promoter. Electroporation and selection for kanamycin resistance was as described (17) except cre was induced for only 1 hr with 0.20% L-arabinose. Resulting Ap^{R} Cm^{R} Kn^{R} colonies were pooled, plasmid DNA was purified and, to minimize contamination by carryover, the selection procedure was repeated two more times. Digestion of DNA with NcoI before retransformation eliminated the lox⁺³ plasmid while retaining the mutant Cre-expressing plasmids that have no NcoI sites. In the absence of cre the frequency of Kn^{R} colonies was less than 1×10⁻⁵.

For the initial round of selection the number of independent mutants subjected for selection was 10⁹, which is in about 10⁶ excess of theoretical maximum of library complexity. This excess was used to ensure that all the mutants having either low representation or low activity on the lox⁺³ substrate were not going to be lost by chance. Due to enrichment, during further rounds the number of independent plasmids subjected to selection (transformation rate) gradually decreased from 10⁸ to 10⁵.

In Vivo Inversion Assay

The in vivo inversion assay was based on ability to invert a lac promoter-containing fragment flanked with two lox⁺³ sites in opposite orientation. Cre mediated inversion of this fragment flips the orientation of the lac promoter from default to the lacZa coding sequence, thus allowing expression of lacZa to occur. Briefly, mutant Cre-expressing plasmids were electroporated into DH5a [pBS1040], where pBS1040 is a pACYC-based Cm^{R} plasmid carrying the fragment flanked with two lox⁺³ sites in opposite orientation containing the lac promoter oriented out of lacZa coding sequence. Transformation and Cre induction was done as described above. To score the inversion rate, cells were grown on plates containing the appropriate antibiotics (ampicillin for Cre expressing plasmid, chloramphenicol for reporter plasmid) and X-gal. Inversion results in expression of lacZa gene and thus results in a blue coloring of colonies.

Protein Purification

Cre protein and its mutants were expressed to high levels in E. coli BL21(DE3) LysS using a T7 expression system, and then purified to homogeneity and stored as described previously (17). The concentrations of wt and mutant Cre proteins were determined by spectrophotometry at 280 nm using an ε₂₈₀ for wt Cre of 1.17×10⁻⁵ M⁻¹ cm⁻¹ (18). Cre was diluted to a working concentration of 1 μM in 20 mM Tris-HCl pH8.0, 1 M NaCl, 1 mM EDTA, 25% glycerol and 100 ng/μl BSA prior to use in vitro.

Recombination In Vitro

For recombination in vitro, the 6.8 kb lox⁺³ pBS835 was cleaved with BglI and NotI to generate two DNA fragments (4.2 and 2.6 kb) with one lox⁺³ site per fragment. Intermolecular recombination between lox⁺³ sites yields two DNA fragments (5.5 and 1.3 kb) readily distinguishable from the substrate fragments by size. All recombination reactions were in a 12 μl reaction volume containing Cre reaction buffer (50 mM Tris-HCl pH 7.5, 140 mM NaCl, 10 mM MgCl₂), 2 nM (100 ng) DNA substrate and 83 nM of Cre. Reactions were incubated at 37° C. for 1 hour, terminated by phenol/chloroform extraction and ethanol precipitation, and analyzed by electrophoresis in 1% agarose gels.

Binding Assay

Binding was measured by an electrophoretic mobility shift assay (EMSA), as described previously (19). Because the cooperativity of Cre binding to lox depends critically on the reaction conditions used (20) we took care to use the same conditions for DNA binding as were used for in vitro DNA recombination. As a single lox DNA substrate we used a 158-162 bp PCR fragment of a subject plasmid, corresponding to each of the mutant or wt lox sites, 5′-[³²P]-labeled with T4 polynucleotide kinase. DNA binding reactions were carried out in a 12 μl reaction volume containing LMD buffer, 83 ng/μl BSA, 8.3 ng/μl calf thymus DNA, 0.05 nM (0.06 ng) of the ³²P-labelled DNA substrate and Cre (0-30 nM). Reactions were incubated at 37° C. for 30 minutes. After incubation 2 μl of loading buffer was added and samples immediately loaded on a pre-run 6% native polyacrylamide gel. Gels were quantified using a PhosphorImager (Molecular Dynamics, Sunnyvale, Calif.) scanner.

Equilibrium binding constants were determined by fitting K_{A1} and K_{A2} parameters of the following equation to quantified data from two independent EMSA experiments, where s is the fraction of free unbound DNA substrate, c1 is the fraction of DNA substrate bound with one Cre subunit, c2 is the fraction of DNA substrate bound with two Cre subunits.

$$s = \frac{1}{1 + {K}_{A 1} [ Cre ] + {K}_{A 1} {{K}_{A 2} [ Cre ]}^{2}}$$

$$c 1 = \frac{{K}_{A 1} [ Cre ]}{1 + {K}_{A 1} [ Cre ] + {K}_{A 1} {{K}_{A 2} [ Cre ]}^{2}}$$

$$c 2 = \frac{{K}_{A1} {{K}_{A2} [ Cre ]}^{2}}{1 + {K}_{A 1} [ Cre ] + {K}_{A 1} {{K}_{A 2} [ Cre ]}^{2}}$$

DNA Cleavage Assays

For the intact loxP and lox⁺³ substrates, the oligonucleotide KC335 (TCG AGT GCA CAA CTT CGT ATA ATG TAT GCT ATA CGA AGT TAT CAT TCG CTA G (SEQ. ID NO. 141) and KC341 (TCG AGT GCA CAA CTT CGT ATA ATG ATT TAT GCT ATA CGA AGT TAT CAT TCG CTA G (SEQ. ID NO. 142) were 5′ labeled with [γ-³²P] ATP using T4 polynucleotide kinase, annealed with the complementary oligonucleotides.

For the nicked loxP cleavage substrate oligonucleotide KC319 (GTG CAC AAC TTC GTA TAA T (SEQ. ID NO. 143) was labeled as above and annealed with both KC322 (GTA TGC TAT ACG AAG TTA TCA TTC GCT AG (SEQ. ID NO. 144) and KC325 (GAT TTA TGC TAT ACG AAG TTA TCA TTC GCT AG (SEQ. ID NO. 145).

DNA cleavage reactions were in a 12 μl reaction volume containing Cre reaction buffer, 83 ng/μl BSA, 8.3 ng/μl calf thymus DNA, 2 mM appropriate ³²P-labeled DNA substrate and 30 nM of Cre. Reactions were incubated at 37° C. for 1 hour, terminated by addition of 12 μl of 2×SDS-gels loading buffer (x: 40 mM Tris-HCl pH 6.8, 50 mM DTT, 1.0% SDS, 7.5% Glycerol, 0.01% Bromphenol Blue), heated at 95° C. for 5 minutes and then analyzed by 15% SDS-PAGE. Gels were quantified using a PhosphorImager (Molecular Dynamics, Sunnyvale, Calif.) scanner.

Synaptic Complex Formation

As substrates for synapse formation assay base pairs 536-544 that were ³²P-labeled DNA fragments with two identical wt or mutant lox sites in inverted orientation located 33 and 56 bp from the ends of the fragments were utilized. Substrates were generated by PCR from the corresponding lox inversion plasmids. To block Cre-mediated catalysis and recombination CreY324F mutant protein was used instead of wt Cre and was performed exactly as for the binding assay. Reaction mixtures were analyzed by non-denaturing 3.5% PAGE.

### Example 1

### Isolation of cre Mutants Active at lox⁺³

A library of random pentapeptide insertions in an arabinose-inducible cre gene by in vitro Mu transposition was constructed as described above. Transposition and subsequent deletion of the Mu transposon resulted in a net 15 bp insertion to give a 5 amino acid insertion into the protein product. The library had >75-fold coverage with insertions targeted to a 1067 bp HindIII-XbaI fragment carrying the 1029 bp cre gene.

Using the insertion library cre mutants were selected that recombine lox⁺³ sites based on their ability in E. coli to excise a transcription terminator flanked by two directly repeated lox⁺³ sites (FIG. 1). Excision releases the block to neo gene expression, thus conferring kanamycin resistance. Whereas transformation of a similarly configured strain carrying a loxP-flanked terminator with wt cre gave 86% recombination, transformation of the lox⁺³ strain with the cre insertion library resulted in a Kn^{R} frequency of 3×10⁻⁴ (FIG. 1), slightly less than the 1×10⁻³ frequency observed with wt cre. To enrich for lox⁺³ active cre mutants present in the insertion library plasmid, DNA from several thousand colonies was pooled and retransformed into the lox⁺³ strain, again selecting for Kn^{R}. After 7 cycles of enrichment recombination of the lox⁺³ substrate by the population of insertion mutants was robust, giving 80% Kn^{R} or nearly the same frequency obtained with wt cre and a loxP substrate (FIG. 1).

To monitor the enrichment of lox⁺³ active cre mutants the cre gene from Kn^{R} isolates obtained after either four or seven cycles of enrichment was sequenced. After four cycles of enrichment 41 of 71 isolates carried insertions within the cre coding region. This increased to 78 of 90 Kn^{R} isolates after seven cycles of enrichment. Table 1 shows that of 41, 4^{th} cycle mutants there were only seven sites of insertion into the 343 amino acid Cre protein; after seven cycles only four of these same insertion site mutants were found among 78 isolates. Of the four mutants obtained after the seventh enrichment cycle, two were represented by only a single isolate. Positionally, the insertion mutants fell into two classes: those located at the N-terminus-helix A region of Cre, and those lying within the loop between the J and K helices. The two predominantly occurring insertion mutants were 286::LRPHW (corresponding to SEQ ID NO. 5; named by the amino acid position of insertion followed by the pentapeptide inserted) and 18::CGRNA (corresponding to SEQ ID NO. 2) where the 18::CGRNA insertion was found in all isolates always to be accompanied by a P15L amino acid change.

TABLE 1
Frequency distribution of mutants selected for lox⁺³ activity.

| | | Number of isolates (% of total) | | | |
|---|---|---|---|---|---|
| | | 4^{th} Enrichment | | 7^{th} Enrichment | |
| Cre mutant | SEQ. ID NO. | Cycle | | Cycle | |
| 18::CGRNA + P15L | 2 | 18 | (44%) | 21 | (27%) |
| 24::CGRIR | 3 | 6 | (15%) | 1 | (1%) |
| 278::CGRND | 146 | 1 | (2%) | 0 | |
| 279::VRPHS | 147 | 1 | (2%) | 0 | |
| 279::GAAAS | 148 | 1 | (2%) | 0 | |
| 280::CGRTG | 4 | 1 | (2%) | 1 | (1%) |
| 286::LRPHW | 5 | 13 | (32%) | 55 | (71%) |
| Total (100%): | | 41 | | 78 | |

### Example 2

### Recombination In Vivo with Individual Cre Mutants

Individual testing of the four mutants obtained after seven enrichment cycles (18::CGRNA+P15L having SEQ ID NO: 2, 24::CGRIR having SEQ ID NO. 3, 280::CGRTG having SEQ ID NO. 4 and 286::LRPHW having SEQ ID NO. 5) showed that all recombined lox⁺³ at high efficiency in vivo (see Table 2). Sequencing confirmed that the recombined reporter plasmid of Kn^{R} transformants carried a single lox⁺³ site, as expected from Cre-mediated recombination. A similar frequency of recombination was observed with these cre mutants using lox⁺³ inversion substrates (data not shown), and sequencing of the products confirmed that Cre-mediated recombination was both site-specific and conservative. Separation and individual testing of the component insertion and missense mutations in the Cre 18::CGRNA+P15L (SEQ ID NO. 2) double mutant showed that both mutations were required for maximal lox⁺³ recombination. Surprisingly, the insertion did not itself increase the efficiency of recombination on a lox⁺³ site even though the nearby 24::CGRIR (SEQ ID NO. 3) insertion was quite active. Instead the missense mutant P15L gave a 60-fold increase in lox⁺³ recombination, and acted synergistically with the 18::CGRNA insertion to give a 560-fold increase in recombination with the lox⁺³ site.

TABLE IIA
IN VIVO RECOMBINATION EFFICIENCIES OF
INDIVIDUAL MUTANTS AT LOX+3 SITES

| | | % Recombination |
|---|---|---|
| Cre mutant | SEQ. ID NO. | (Kn^{R}) |
| 286::LRPHW | 5 | 92% |
| 18::CGRNA + P15L | 2 | 56% |
| 18::CGRNA | 149 | 0.1% |
| P15L | 150 | 6% |
| 24::CGRIR | 3 | 94% |
| 280::CGRTG | 4 | 61% |
| wt | 1 | 0.1% |
| bgr | | 0.001% |

TABLE IIB
RECOMBINATION EFFICIENCIES FOR INDIVIDUAL
MUTANTS AT LOX⁺³ AND LOX⁺⁵ SITES

% Recombination (Kn^{R})

| Cre mutant | lox+3 | lox+5 |
|---|---|---|
| 286::LRPHW | 92% | 0.2% |
| 18::CGRNA + P15L | 56% | 1.4% |
| wt | 0.1% | 0.009% |
| no cre (empty vector) | 0.001% | 0.002% |

### Example 3

### Recombination In Vitro

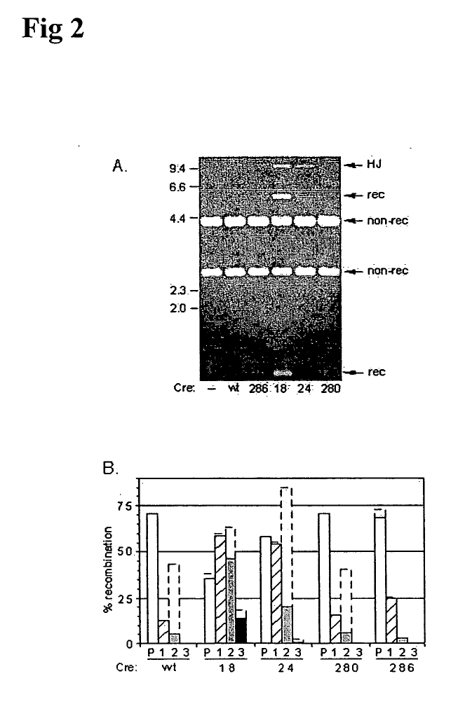

Cre protein was purified to near homogeneity from each of the four mutants present after seven enrichment cycles: 18::CGRNA+P15L (SEQ ID NO. 2), 24::CGRIR (SEQ ID NO. 3), 280::CGRTG (SEQ ID NO. 4) and 286::LRPHW (SEQ ID NO. 5). Incubation with a lox⁺³ excision substrate showed that the two N-terminal insertion mutants gave both the predicted recombination products and also a Holliday junction product (FIG. 2). No recombinant products were obtained with the two J-K loop insertion mutants or with wt Cre. A trace of Holliday junction could be observed with 286::LRPHW (SEQ ID NO. 5).

The wt Cre protein does not recombine lox⁺³ sites, but does display a low amount of recombination activity on both lox⁺¹ and lox⁺² recombination sites (data not shown). Therefore, the activity of the four mutant proteins was compared with wt Cre not only on lox⁺³ but also on lox⁺², lox⁺¹ and wt loxP (FIG. 2). All mutants were active on the wt loxP substrate. Of the two J-K loop mutants, the insertion at position 280 was indistinguishable from wt Cre; whereas the insertion at position 286 showed a two-fold increase of activity with lox⁺¹. The 286 insertion also did not accumulate the Holliday junction products with the lox⁺² substrate observed with wt Cre. In contrast, both N-terminal mutants showed a distinct shift in recombination proficiency to lox sites having an extended spacer, with the A-helix insertion at position 24 showing equivalent recombination efficiency with wt loxP and lox⁺¹ substrates and the 18:: CGRNA+P15L (SEQ ID NO. 2) double mutant distinctly preferring both the lox⁺¹ and lox⁺² sites over wt loxP.

### Example 4

### DNA Binding, Cleavage and Synapsis

It has previously been shown that wt Cre's interaction with the lox⁺³ site is abnormal in three ways: 1) Cre's cooperativity of DNA binding to lox⁺³ is lost and, in fact, becomes negative; 2) Cre-mediated cleavage is strongly reduced on an intact lox⁺³ substrate although cleavage on a nicked or suicide substrate is unaffected; and 3) DNA synapsis with a catalytically inactive Cre protein is abolished (Petyuk and Sauer, (2004) Cre-Mediated Recombination with Spacer Length Mutants of loxP, JBC, submitted for publication). The ability of the isolated Cre mutants to compensate for one of these abnormalities was examined.

Binding of Cre to the lox⁺³ site was evaluated using a gel shift assay. All mutant Cre proteins bound to the lox⁺³ site forming both a c1 complex (one subunit per site) and a c2 complex (two Cre subunits per site). Calculation of the equilibrium constants for each mutant protein showed that all of the mutants bound to the lox⁺³ site with an affinity indistinguishable from that of wt Cre (Table 3) In particular, none of the mutants showed any improvement in the cooperativity of binding to the mutant lox site and all exhibited the same negative cooperativity of binding displayed by wt Cre.

TABLE 3
Equilibrium binding constants of mutant Cre proteins at the lox⁺³ site.

| Cre | K_{A1} (M⁻¹) × 10⁻⁹ | K_{A2} (M⁻¹) × 10⁻⁹ | Cooperatively^{a} |
|---|---|---|---|
| wt | 0.60 ± 0.11 | 0.21 ± 0.04 | 0.35 (0.24-0.51) |
| 993 | 0.74 ± 0.24 | 0.15 ± 0.03 | 0.20 (0.12-0.36) |
| 1010 | 0.92 ± 0.34 | 0.21 ± 0.05 | 0.23 (0.13-0.45) |
| 1011 | 0.93 ± 0.38 | 0.23 ± 0.06 | 0.25 (0.13-0.53) |
| 1012 | 0.60 ± 0.20 | 0.14 ± 0.03 | 0.23 (0.14-0.43) |

^{a}Cooperatively is calculated as the ratio K_{A2}/K_{A1}. Shown in parentheses is the range of this value based on the error in measurement for K_{A1} and K_{A2}.

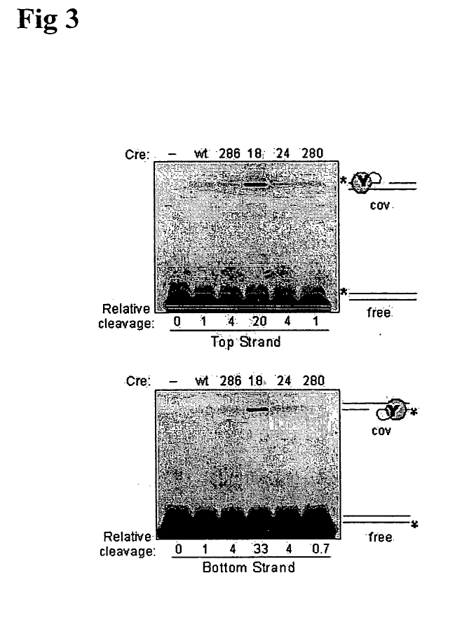

The ability of each mutant to catalyze cleavage at the lox⁺³ site on an unnicked 54 bp substrate was examined. Cleavage by Cre is easily detected as a slowly moving band on a polyacrylamide gel resulting from the formation of a protein-DNA complex in which the catalytic tyrosine of Cre is covalently linked to a 3′ phosphate of the labeled DNA substrate at the site of cleavage (FIG. 3). Using a labled top strand lox⁺³ substrate two mutants, the insertions at positions (286 SEQ ID NO. 5) and 24 (SEQ ID NO. 3), showed a four-fold stimulation of covalent complex formation and one, 18::CGRNA+P15L (SEQ ID NO. 2), showed a 20-fold stimulation. No enhanced cleavage was observed with the insertion mutant 280::CTRTG (SEQ ID NO. 4). Although cleavage of the bottom strand was less efficient than for the top strand, consistent with Cre's strand preference for cleavage, the results clearly showed that the mutant Cre proteins gave the same pattern of enhanced cleavage as was seen with the top strand substrate.

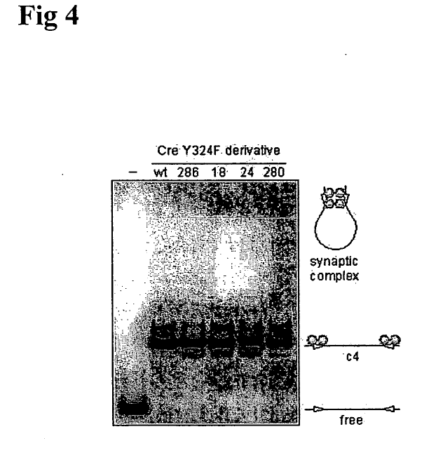

To determine whether any of mutant Cre proteins had regained the ability to promote synapsis at the lox⁺³ site, the catalytic tyrosine at position 324 was mutated to phenylalanine in each of the four insertion mutants and then purified each catalytically inactive compound mutant protein to homogeneity. Use of the catalytically inactive mutant derivatives allowed direct determination of synaptic complex formation by eliminating formation of Holliday junction intermediates and Cre-bound recombination products that migrate at the same position as the synaptic complex. To facilitate synaptic complex formation, a DNA substrate was used having two lox sites on the same molecule. Intramolecular association of the two lox sites by Cre loops the substrate DNA to give a large shift in electrophoretic mobility compared to the “linear” form of the complex having two Cre-occupied lox⁺³ sites do not interact with each other. FIG. 4 shows that no synaptic complex was formed with the wt Cre Y324F derivative. Similarly, no synaptic complex formation was detected with either of the two J-K loop insertion mutants (at positions 280 and 286). In contrast, both N-terminal insertional mutants, 18::CGRNA+P15L and 24::CGRIR, clearly promoted synaptic complex formation and to similar extents. Thus, Cre can gain the ability to promote synaptic complex formation with a lox⁺³ site by mutational alteration either within the A-helix of Cre or just N-terminal of the A-helix.

Discussion

The results obtained show two classes of mutants: one contains insertions at the N-terminus of helix A, and the second has insertions into the J-K loop. Of two types, mutants containing insertions at the N-terminus of the helix A (18::CGRNA,P15L (SEQ ID NO. 2); 24::CGRIR (SEQ ID NO. 3)) confirmed their activity on lox⁺³ sites by recombination in vitro. Due to the lack of selective pressure against activity on wt loxP site, all of the selected mutants can recombine at the loxP site, implying relaxed specificity.

The most active mutant, 18::CGRNA,P15L (SEQ ID NO. 2), has a preference for a longer spacer substrates i.e. it is more active on lox⁺¹ and lox⁺² than on wt loxP, implying that recognition specificity shifted towards longer spacers. Assuming that the size of overlap region in the recognition site is the optimal size for the corresponding integrase/recombinase with such a preferable recognition of the lox⁺¹ site with 7 bp in overlap region, the 18::CGRNA,P15L (SEQ ID NO. 2) mutant most resembles other well studied tyrosine integrases/recombinases from phages. The N-terminal region is the least conserved and, moreover, is structurally and functionally very different within the members of this family. Despite the vast number of well studied examples of tyrosine integrase/recombinase family members, there has been no obvious structural motif detected that may control the spacer length.

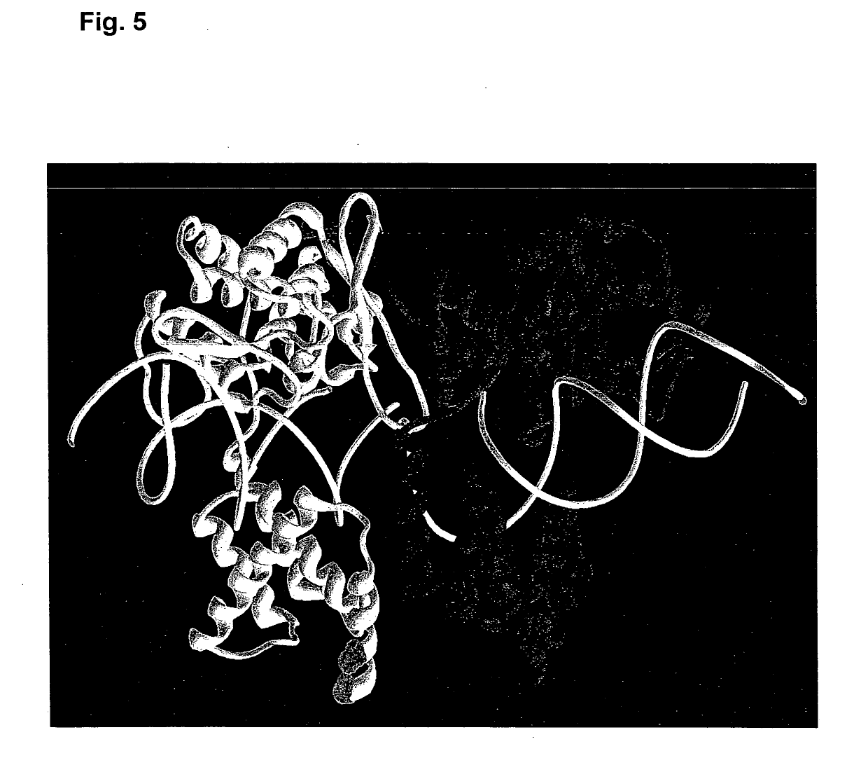

It has been shown that the mutants with insertions at the N-terminus of helix A restore the activity on lox⁺³ site through restoration of the synapse complex. It has been shown that the N-terminal of helix A is involved in protein-protein interactions. Taken together, although the crystal structure of the very N-terminus of Cre is not solved, it is reasonable to assume that this region is in proximity to the neighboring subunit (FIG. 5) and thus, may facilitate intersubunit interactions.

The other phenotype observed, which distinguishes these insertion mutants from wt Cre, is an enhanced cleavage of DNA substrate. Due to an accumulation of covalently attached Cre-DNA intermediate, it is evident that cleavage is especially prominent for bottom strand (FIG. 3). Enhancement of accumulation of covalently attached product may be achieved by several means: enhancement of cleavage by synapse complex formation, enhancement of cleavage catalysis in synapse-independent manner and stabilization of cleaved products. Thus, the enhanced cleavage may be additional evidence of enhanced synapse complex formation.

Moreover, despite the fact that this mutation restored the synapse complex it did not restore the cooperativity of binding and did not facilitate formation of c2 complex. Hence, the cooperativity of binding is not necessary for restoration of synapse and recombination. This data provides evidence that the protein-protein interactions governing synapse are not equivalent to those governing cooperativity.

Also, the mere existence of another type of mutant, i.e. insertional mutants into the J-K loop, suggests that there is another way to overcome problems with recombination of lox⁺³ site other than remodeling the N-terminus of helix A. Unlike the results obtained in vivo, recombination of the lox⁺³ substrate by this type of mutant under standard conditions in vitro was not detected. Although the 280::CGRTG (SEQ ID NO. 4) mutant recombined all the substrates tested at the same efficiency as wt Cre, the 286::LRPHW (SEQ ID NO. 5) mutant is slightly more active on a lox⁺¹ substrate and a on lox⁺² substrate, the mutant does not efficiently resolve the HJ intermediate. For in vitro activity of mutants containing insertions at the J-K loop on lox⁺³ substrate, it is possible that some environment or additional factors present in vivo are required.

All references cited in the preceding text of the patent application or in the following reference list, to the extent that they provide exemplary, procedural, or other details supplementary to those set forth herein, are specifically incorporated by reference to the same extent as if each individual publication or patent application was specifically and individually indicated to be incorporated by reference

### REFERENCES

1. Azaro, M. A., and Landy, A. (2002) in Mobile DNA II (Craig, N. L., Craigie, R., Gellert, M., and Lambowitz, A. M., eds), pp. 118-148, ASM Press, Washington, D.C.
2. Van Duyne, G. D. (2002) in Mobile DNA II (Craig, N. L., Craigie, R., Gellert, M., and Lambowitz, A. M., eds), pp. 93-117, ASM Press, Washington, D.C.
3. Barre, F.-X., and Sherratt, D. J. (2002) in Mobile DNA II (Craig, N. L., Craigie, R., Gellert, M., and Lambowitz, A. M., eds), ASM Press, Washington, D.C.
4. Hoess, R. H., Wierzbicki, A., and Abremski, K. (1986) Nucleic Acids Res. 14, 2287-2300
5. Lee, G., and Saito, I. (1998) Gene 216, 55-65
6. Senecoff, J. F., Bruckner, R. C., and Cox, M. M. (1985) Proc. Natl. Acad. Sci. USA 82, 7270-7274
7. de Massy, B., Studier, F. W., Dorgai, L., Appelbaum, E., and Weisberg, R. A. (1984) Cold Spring Harb. Symp. Quant. Biol. 49, 715-726
8. Shulman, M., and Gottesman, M. (1973) J. Mol. Biol. 81, 461-482
9. Sternberg, N., Hamilton, D., and Hoess, R. (1981) J. Mol. Biol. 150, 487-507
10. Hoess, R. H., Ziese, M., and Sternberg, N. (1982) Proc. Natl. Acad. Sci. USA 79, 3398-3402
11. Sauer, B. (1992) J. Mol. Biol. 223, 911-928
12. Rüfer, A. W., and Sauer, B. (2002) Nucleic Acids Res. 30, 2764-2771
13. Sauer, B., Whealy, M., Robbins, A., and Enquist, L. (1987) Proc. Natl. Acad. Sci. USA 84, 9108-9112
14. Guzman, L.-M., Belin, D., Carson, M. J., and Beckwith, J. (1995) J. Bacteriol. 177, 4121-4130
15. Cao, Y., Hallet, B., Sherratt, D. J., and Hayes, F. (1997) J Mol Biol 274, 39-53
16. Poussu, E., Vihinen, M., Paulin, L., and Savilahti, H. (2004) Proteins 54, 681-692
17. Biery, M. C., Stewart, F. J., Stellwagen, A. E., Raleigh, E. A., and Craig, N. L. (2000) Nucleic Acids Res 28, 1067-1077
18. Haapa, S., Taira, S., Heikkinen, E., and Savilahti, H. (1999) Nucleic Acids Res 27, 2777-2784
19. Ringrose, L., Lounnas, V., Ehrlich, L., Buchholz, F., Wade, R., and Stewart, A. F. (1998) J. Mol. Biol. 284, 363-384
20. Guo, F., Gopaul, D. N., and Van Duyne, G. D. (1999) Proc. Natl. Acad. Sci. USA 96, 7143-7148
21. Hartung, M. & Kisters-Woike, B. (1998) J. Biol. Chem. 273, 22884-22891.
22. Buchholz, F. & Stewart, A. F. (2001) Nat. Biotechnol. 19, 1047-1052.
23. Santoro, S. W. & Schultz, P. G. (2002) Proc. Natl. Acad. Sci. USA 99, 4185-4190.
24. Rüfer, A. W. & Sauer, B. (2002) Nucleic Acids Res. 30, 2764-2771.
25. Petyuk, V., McDermott, J., Cook, M. & Sauer, B. (2004) J Biol Chem in press.
26. Guo, F., Gopaul, D. N. & Van Duyne, G. D. (1997) Nature 389, 40-46.

What is claimed:

1. A purified Cre mutant polypeptide, the mutant polypeptide having an amino acid sequence such that it specifically binds to an antibody that binds specifically to a Cre wild-type polypeptide having SEQ ID NO. 1, wherein the Cre mutant polypeptide can catalyze site specific recombination at a lox site having at least one additional nucleotide in the spacer region.

2. The purified Cre mutant polypeptide of claim 1, the amino acid sequence of which comprises a sequence at least 50% identical to SEQ ID NO. 1.

3. The purified Cre mutant polypeptide of claim 1, the amino acid sequence of which comprises a sequence at least 75% identical to SEQ ID NO. 1.

4. The purified Cre mutant polypeptide of claim 1, the amino acid sequence of which comprises a sequence at least 90% identical to SEQ ID NO. 1.

5. The purified Cre mutant polypeptide of claim 1, the amino acid sequence of which comprises a sequence at least 95% identical to SEQ ID NO. 1.

6. The purified Cre mutant polypeptide of claim 1, the amino acid sequence of which comprises a sequence at least 99% identical to SEQ ID NO. 1.

7. The purified Cre mutant polypeptide of claim 1, the amino acid sequence of which comprises SEQ ID NO. 1 with 1 to 50 conservative amino acid substitutions.

8. The purified Cre mutant polypeptide of claim 1, the amino acid sequence of which comprises SEQ ID NO. 1 with 1 to 15 conservative amino acid substitutions.

9. The purified Cre mutant polypeptide of claim 1, the amino acid sequence of which comprises SEQ ID NO. 1 with 1 to 10 conservative amino acid substitutions.

10. The purified Cre mutant polypeptide of claim 1, the amino acid sequence of which comprises SEQ ID NO. 1 with 5 additional amino acids inserted consecutively within the N-terminus of helix A.

11. The purified Cre mutant polypeptide of claim 10, wherein the 5 additional amino acids are inserted after either residue 18 or residue 24 of SEQ ID NO. 1.

12. The purified Cre mutant polypeptide of claim 1, the amino acid sequence of which comprises SEQ ID NO. 1 with 5 additional amino acids inserted consecutively within the J-K loop.

13. The purified Cre mutant polypeptide of claim 12, wherein the 5 additional amino acids are inserted after either residue 280 or 286 of SEQ ID NO. 1.

14. A purified Cre mutant polypeptide, the amino acid sequence of which comprises SEQ ID NO. 1 with 5 additional amino acids inserted consecutively within the N-terminus of helix A, wherein the Cre mutant polypeptide can catalyze site specific recombination at a lox site having at least one additional nucleotide in the spacer region.

15. The purified Cre mutant polypeptide of claim 14, wherein the 5 additional amino acids are inserted after either residue 18 or residue 24 of SEQ ID NO. 1.

16. The purified Cre mutant polypeptide of claim 14, the amino acid sequence of which is selected from the group consisting of SEQ ID. nos. 2 and 3.

17. The purified Cre mutant polypeptide of claim 16, the amino acid sequence of which comprises a sequence at least 50% identical to either SEQ ID. NO. 2 or 3.

18. The purified Cre mutant polypeptide of claim 16, the amino acid sequence of which comprises a sequence at least 75% identical to either SEQ ID. NO. 2 or 3.

19. The purified Cre mutant polypeptide of claim 16, the amino acid sequence of which comprises a sequence at least 90% identical to either SEQ ID. NO. 2 or 3.

20. The purified Cre mutant polypeptide of claim 16, the amino acid sequence of which comprises a sequence at least 95% identical to either SEQ ID. NO. 2 or 3.

21. The purified Cre mutant polypeptide of claim 16, the amino acid sequence of which comprises a sequence at least 99% identical to either SEQ ID. NO. 2 or 3.

22. The purified Cre mutant polypeptide of claim 16, the amino acid sequence of which comprises either SEQ ID NO. 2 or 3 with 1 to 50 conservative amino acid substitutions.

23. The purified Cre mutant polypeptide of claim 16, the amino acid sequence of which comprises SEQ ID NO. 2 or 3 with 1 to 15 conservative amino acid substitutions.

24. The purified Cre mutant polypeptide of claim 16, the amino acid sequence of which comprises SEQ ID NO. 2 or 3 with 1 to 10 conservative amino acid substitutions.

25. The purified Cre mutant polypeptide of claim 14, the mutant polypeptide having an amino acid sequence such that it specifically binds to an antibody that binds specifically to a polypeptide having either SEQ ID NO. 2 or 3.

26. A purified Cre mutant polypeptide, the amino acid sequence of which comprises SEQ ID NO. 1 with 5 additional amino acids inserted consecutively within the J-K loop, wherein the Cre mutant polypeptide can catalyze site specific recombination at a lox site having at least one additional nucleotide in the spacer region.

27. The purified Cre mutant polypeptide of claim 26, wherein the 5 additional amino acids are inserted after either residue 280 or residue 286 of SEQ ID NO. 1.

28. The purified Cre mutant polypeptide of claim 26, the amino acid sequence of which is selected from the group consisting of SEQ ID. nos. 4 and 5.

29. The purified Cre mutant polypeptide of claim 28, the amino acid sequence of which comprises a sequence at least 50% identical to either SEQ ID. NO. 4 or 5.

30. The purified Cre mutant polypeptide of claim 28, the amino acid sequence of which comprises a sequence at least 75% identical to either SEQ ID. NO. 4 or 5.

31. The purified Cre mutant polypeptide of claim 28, the amino acid sequence of which comprises a sequence at least 90% identical to either SEQ ID. NO. 4 or 5.

32. The purified Cre mutant polypeptide of claim 28, the amino acid sequence of which comprises a sequence at least 95% identical to either SEQ ID. NO. 4 or 5.

33. The purified Cre mutant polypeptide of claim 28, the amino acid sequence of which comprises a sequence at least 99% identical to either SEQ ID. NO. 4 or 5.

34. The purified Cre mutant polypeptide of claim 28, the amino acid sequence of which comprises either SEQ ID NO. 4 or 5 with 1 to 50 conservative amino acid substitutions.

35. The purified Cre mutant polypeptide of claim 28, the amino acid sequence of which comprises SEQ ID NO. 4 or 5 with 1 to 15 conservative amino acid substitutions.

36. The purified Cre mutant polypeptide of claim 28, the amino acid sequence of which comprises SEQ ID NO. 4 or 5 with 1 to 10 conservative amino acid substitutions.

37. The purified Cre mutant polypeptide of claim 26, the mutant polypeptide having an amino acid sequence such that it specifically binds to an antibody that binds specifically to a polypeptide having either SEQ ID NO. 4 or 5.

38. A purified Cre mutant polypeptide, the mutant polypeptide having an amino acid sequence such that it specifically binds to an antibody that binds specifically to a polypeptide having a sequence selected from the group consisting of SEQ ID nos. 6-17, wherein the Cre mutant polypeptide can catalyze site specific recombination at a lox site having at least one additional nucleotide in the spacer region.

39. A purified antibody that binds specifically to a polypeptide having an amino acid sequence selected from the group consisting of SEQ ID nos. 2-5.

40. The purified antibody of claim 39, wherein the antibody is a monoclonal or polyclonal antibody.

41. The purified antibody of claim 39, wherein the antibody is a variant selected from the group consisting of a single chain recombinant antibody, a humanized chimeric antibody, a Fab fragment antibody, and a Fab′ fragment antibody.

42. A method of making an antibody, comprising immunizing a non-human animal with an immunogenic fragment of a polypeptide having an amino acid sequence selected from the group consisting of SEQ ID nos. 2-5.

43. A method of purifying a polypeptide having an amino acid sequence selected from the group consisting of SEQ ID nos. 2-5 from a biological sample containing the polypeptide, the method comprising:
(a) providing an affinity matrix comprising the antibody of claim 39 bound to a solid support;
(b) contacting the biological sample with the affinity matrix, to produce an affinity matrix-polypeptide complex;
(c) separating the affinity matrix-polypeptide complex from the remainder of the biological sample; and
(d) releasing the polypeptide from the affinity matrix.

44. An isolated nucleotide sequence comprising a sequence that encodes a polypeptide having an amino acid sequence selected from the group consisting of SEQ ID nos. 2-5, or of a fragment of any of SEQ ID nos. 2-5 that is at least 15 amino acid residues in length.

45. The isolated nucleotide sequence of claim 44, wherein the nucleotide sequence encodes a Cre mutant polypeptide that can catalyze site specific recombination at a lox site having at least one additional nucleotide in the spacer region.

46. The isolated nucleotide sequence of claim 44, wherein the nucleotide sequence encodes a polypeptide having at least one conservative amino acid substitution.

47. The isolated nucleotide sequence of claim 46, wherein the nucleotide sequence encodes a Cre mutant polypeptide that can catalyze site specific recombination at a lox site having at least one additional nucleotide in the spacer region.

48. The isolated nucleotide sequence of claim 44, wherein the nucleotide sequence comprises a sequence that encodes a polypeptide having an amino acid sequence that is at least 50% identical to an amino acid sequence selected from the group consisting of SEQ ID nos. 2-5.

49. The isolated nucleotide sequence of claim 48, wherein the nucleotide sequence encodes a Cre mutant polypeptide that can catalyze site specific recombination at a lox site having at least one additional nucleotide in the spacer region.

50. The isolated nucleotide sequence of claim 44, wherein the nucleotide sequence comprises a sequence that encodes a polypeptide having an amino acid sequence that is at least 75% identical to an amino acid sequence selected from the group consisting of SEQ ID nos. 2-5.

51. The isolated nucleotide sequence of claim 50, wherein the nucleotide sequence encodes a Cre mutant polypeptide that can catalyze site specific recombination at a lox site having at least one additional nucleotide in the spacer region.

52. The isolated nucleotide sequence of claim 44, wherein the nucleotide sequence comprises a sequence that encodes a polypeptide having an amino acid sequence that is at least 95% identical to an amino acid sequence selected from the group consisting of SEQ ID nos. 2-5.

53. The isolated nucleotide sequence of claim 52, wherein the nucleotide sequence encodes a Cre mutant polypeptide that can catalyze site specific recombination at a lox site having at least one additional nucleotide in the spacer region.

54. The isolated nucleotide sequence of claim 44, wherein the nucleotide sequence comprises a sequence that encodes a polypeptide having an amino acid sequence that is at least 99% identical to an amino acid sequence selected from the group consisting of SEQ ID nos. 2-5.

55. The isolated nucleotide sequence of claim 54, wherein the nucleotide sequence encodes a Cre mutant polypeptide that can catalyze site specific recombination at a lox site having at least one additional nucleotide in the spacer region.

56. The isolated nucleotide sequence of claim 44, wherein the nucleotide sequence hybridizes under stringent conditions to a hybridization probe the nucleotide sequence of which encodes a encodes a polypeptide having an amino acid sequence selected from the group consisting of SEQ ID nos. 2-5.

57. The isolated nucleotide sequence of claim 56, wherein the nucleotide sequence encodes a Cre mutant polypeptide that can catalyze site specific recombination at a lox site having at least one additional nucleotides in the spacer region.

58. An expression vector comprising the nucleotide sequence of claim 44 operably linked to a regulatory sequence.

59. A cultured cell comprising the expression vector of claim 58.

60. A cultured cell comprising the nucleotide sequence of claim 44 operably linked to an expression control sequence.

61. A cultured cell transfected with the vector of claim 58, or a progeny of the cell, wherein the cell expresses the polypeptide.

62. A method of producing a polypeptide, the method comprising culturing the cell of claim 59 under conditions permitting the expression of the polypeptide.

63. A method of producing a polypeptide, the method comprising culturing the cell of claim 59 under conditions permitting expression under the control of the regulatory sequence, and purifying the protein from the cell or the medium of the cell.

64. An isolated lox nucleotide sequence comprising a sequence at least 50% identical to SEQ ID. nos. 18 or 139 with three additional nucleotides in the spacer region.

65. The isolated lox nucleotide sequence of claim 64, wherein the sequence is at least 75% identical to SEQ ID nos. 18 or 139.

66. The isolated lox nucleotide sequence of claim 64, wherein the sequence is at least 90% identical to SEQ ID nos. 18 or 139.

67. The isolated lox nucleotide sequence of claim 64, wherein the sequence is at least 95% identical to SEQ ID nos. 18 or 139.

68. The isolated lox nucleotide sequence of claim 64, wherein the sequence is at least 99% identical to SEQ ID nos. 18 or 139.

69. The isolated lox nucleotide sequence of claim 64, wherein the three additional nucleotides in the spacer region are selected from the group consisting of adenosine 5′-monophosphate and thymidine 5′-monophosphate.

70. The isolated lox nucleotide sequence of claim 69, wherein the three additional nucleotides in the spacer region are all adenosine 5′-monophosphate.

71. The isolated lox nucleotide sequence of claim 69, wherein the three additional nucleotides in the spacer region are all thymidine 5′-monophosphate.

72. The isolated lox nucleotide sequence of claim 69, wherein the three additional nucleotides in the spacer region consist of two adenosine 5′-monophosphates and one thymidine 5′-monophosphate.

73. The isolated lox nucleotide sequence of claim 69, wherein the three additional nucleotides in the spacer region consist of one adenosine 5′-monophosphate and two thymidine 5′-monophosphates.

74. The isolated lox nucleotide sequence of claim 64, wherein the three additional nucleotides in the spacer region are selected from the group consisting of guanosine 5′-monophosphate and cytidine 5′-monophosphate.

75. The isolated lox nucleotide sequence of claim 74, wherein the three additional nucleotides in the spacer region are all guanosine 5′-monophosphate.

76. The isolated lox nucleotide sequence of claim 74, wherein the three additional nucleotides in the spacer region are all cytidine 5′-monophosphate.

77. The isolated lox nucleotide sequence of claim 74, wherein the three additional nucleotides in the spacer region consist of two guanosine 5′-monophosphates and one cytidine 5′-monophosphate.

78. The isolated lox nucleotide sequence of claim 74, wherein the three additional nucleotides in the spacer region consist of one guanosine 5′-monophosphate and two cytidine 5′-monophosphates.

79. The isolated lox nucleotide sequence of claim 64, wherein the three additional nucleotides in the spacer region are selected from the group consisting of adenosine 5′-monophosphate, thymidine 5′-monophosphate, guanosine 5′-monophosphate and cytidine 5′-monophosphate.

80. The isolated lox nucleotide sequence of claim 79, wherein the three additional nucleotides in the spacer region consist of two adenosine 5′-monophosphates and one guanosine 5′-monophosphate.

81. The isolated lox nucleotide sequence of claim 79, wherein the three additional nucleotides in the spacer region consist of two adenosine 5′-monophosphates and one cytidine 5′-monophosphate.

82. The isolated lox nucleotide sequence of claim 79, wherein the three additional nucleotides in the spacer region consist of two thymidine 5′-monophosphates and one guanosine 5′-monophosphate.

83. The isolated lox nucleotide sequence of claim 79, wherein the three additional nucleotides in the spacer region consist of two thymidine 5′-monophosphates and one cytidine 5′-monophosphate.

84. The isolated lox nucleotide sequence of claim 79, wherein the three additional nucleotides in the spacer region consist of one thymidine 5′-monophosphate, one adenosine 5′-monophosphate and one cytidine 5′-monophosphate.

85. The isolated lox nucleotide sequence of claim 79, wherein the three additional nucleotides in the spacer region consist of one thymidine 5′-monophosphate, one adenosine 5′-monophosphate and one guanosine 5′-monophosphate.

86. The isolated lox nucleotide sequence of claim 79, wherein the three additional nucleotides in the spacer region consist of one adenosine 5′-monophosphate and two guanosine 5′-monophosphates.

87. The isolated lox nucleotide sequence of claim 79, wherein the three additional nucleotides in the spacer region consist of one thymidine 5′-monophosphate and two guanosine 5′-monophosphates.

88. The isolated lox nucleotide sequence of claim 79, wherein the three additional nucleotides in the spacer region consist of one adenosine 5′-monophosphate and two cytidine 5′-monophosphates.

89. The isolated lox nucleotide sequence of claim 79, wherein the three additional nucleotides in the spacer region consist of one thymidine 5′-monophosphate and two cytidine 5′-monophosphates.

90. The isolated lox nucleotide sequence of claim 79, wherein the three additional nucleotides in the spacer region consist of one thymidine 5′-monophosphate, one guanosine 5′-monophosphate and one cytidine 5′-monophosphate.

91. The isolated lox nucleotide sequence of claim 79, wherein the three additional nucleotides in the spacer region consist of one adenosine 5′-monophosphate, one guanosine 5′-monophosphate and one cytidine 5′-monophosphate.

92. An isolated lox nucleotide sequence having formula (I)
R₁—X—R₁ (I)
wherein:
R₁ is a wild-type inverted repeat region; and
X is a wild-type spacer region with from one to three additional nucleotide base pairs.

93. The isolated lox nucleotide sequence of claim 92, wherein R₁ and X are from loxP

94. An isolated lox nucleotide sequence having formula (II) 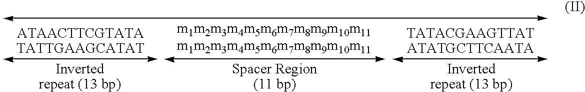
wherein:
m₁, m₂, m₃, m₄, m₅, m₆, m₇, m₈, m₉, m₁₀, and m₁₁ together comprise the spacer region and are independently a complementary nucleotide base pair wherein the nitrogenous base is a purine or a pyrimidine.

95. An isolated lox nucleotide sequence having formula (III) 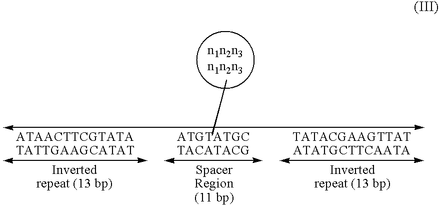
wherein:
n₁, n₂, and n₃ are independently a complementary base pair wherein the nitrogenous base is a purine or pyrimidine.

96. A vector comprising at least two lox nucleotide sequences of claim 64.

97. A vector comprising a first lox nucleotide sequence of claim 64, a second lox nucleotide sequence of claim 64 and a transcriptional terminator, wherein the terminator is located between the first lox nucleotide sequence and the second lox nucleotide sequence.

98. The vector of claim 96, further comprising a marker gene.

99. The vector of claim 98, further comprising a neo gene.

100. A cultured cell comprising the expression vector of claim 96.

101. A cultured cell comprising the expression vector of claim 97.

102. An isolated lox nucleotide sequence comprising a sequence at least 50% identical to SEQ ID. nos. 18 or 139 with two additional nucleotides in the spacer region.

103. The isolated lox nucleotide sequence of claim 102, wherein the sequence is at least 75% identical to SEQ ID nos. 18 or 139.

104. The isolated lox nucleotide sequence of claim 102, wherein the sequence is at least 90% identical to SEQ ID nos. 18 or 139.

105. The isolated lox nucleotide sequence of claim 102, wherein the sequence is at least 95% identical to SEQ ID nos. 18 or 139.

106. The isolated lox nucleotide sequence of claim 102, wherein the sequence is at least 99% identical to SEQ ID nos. 18 or 139.

107. The isolated lox nucleotide sequence of claim 102, wherein the two additional nucleotides in the spacer region are selected from the group consisting of adenosine 5′-monophosphate and thymidine 5′-monophosphate.

108. An isolated lox nucleotide sequence comprising a sequence at least 50% identical to SEQ ID. nos. 18 or 139 with one additional nucleotide in the spacer region.

109. The isolated lox nucleotide sequence of claim 108, wherein the sequence is at least 75% identical to SEQ ID nos. 18 or 139.

110. The isolated lox nucleotide sequence of claim 108, wherein the sequence is at least 90% identical to SEQ ID nos. 18 or 139.

111. The isolated lox nucleotide sequence of claim 108, wherein the sequence is at least 95% identical to SEQ ID nos. 18 or 139.

112. The isolated lox nucleotide sequence of claim 108, wherein the sequence is at least 99% identical to SEQ ID nos. 18 or 139.

113. The isolated lox nucleotide sequence of claim 108, wherein the one additional nucleotide in the spacer region is selected from the group consisting of adenosine 5′-monophosphate and thymidine 5′-monophosphate.

114. A method for producing site-specific recombination in a nucleotide sequence having a target DNA segment, the method comprising:
(a) introducing a first lox site and a second lox site into the nucleotide sequence such that the target DNA segment is flanked by the first and second lox sites, each lox site having from one to three additional nucleotides in the spacer region;
(b) contacting the nucleotide sequence with a Cre mutant polypeptide that can catalyze site specific recombination at a lox site having from one to three additional nucleotides in the spacer region, thereby producing site specific recombination.

115. The method of claim 114, further comprising introducing a nucleotide sequence encoding a mutant Cre polypeptide operably linked to an inducible promoter.

116. The method of claim 114, wherein the first and second lox sites have the same orientation and the site-specific recombination of the nucleotide sequence is a deletion of the target DNA segment.

117. The method of claim 116, wherein the target DNA segment is selected from the group consisting of a gene, a coding region, and a nucleotide sequence that regulates gene expression in a cell.

118. The method of claim 114, wherein the first and second lox sites are loxP.

119. The method of claim 114, wherein the Cre mutant polypeptide is the polypeptide of claim 1.

120. The method of claim 116, wherein the first and second lox sites have opposite orientations and the site specific recombination is an inversion of the nucleotide sequence of the target DNA segment.

121. The method of claim 120, wherein the target DNA segment is selected from the group consisting of a gene, a coding region, and a nucleotide sequence that regulates gene expression in a cell.

122. The method of claim 121, wherein the first and second lox sites are loxP.

123. The method of claim 122, wherein the Cre mutant polypeptide is the polypeptide of claim 1.

124. The method of claim 114, wherein the first and second lox sites are introduced into two different nucleotide sequences and the site-specific recombination is a reciprocal exchange of nucleotide sequence segments proximate to the lox sites.

125. The method of claim 124, wherein the first and second lox sites are loxP.

126. The method of claim 125, wherein the Cre mutant polypeptide is the polypeptide of claim 1.

127. The method of claim 114, wherein the site-specific recombination occurs in a cell that is prokaryotic or eukaryotic.

128. The method of claim 127, wherein the cell is selected from the group consisting of bacterial, mammalian and plant.

129. The method of claim 114, wherein the site-specific recombination occurs in vitro or in vivo.

130. A method of excising a target DNA segment from a nucleic acid sequence in a trangenic non human organism, the method comprising:
(a) introducing into a cell of the organism a first lox site and a second lox site, the second lox site being in the same orientation as the first lox site, each lox site having from one to three additional nucleotides in the spacer region, wherein the lox sites flank the target DNA segment;
(b) contacting the nucleotide sequence comprising the lox sites flanked by the target DNA segment with a Cre mutant polypeptide that can catalyze site specific recombination at a lox site having from one to three additional nucleotides in the spacer region, thereby excising the target DNA segment.

131. The method of claim 130, wherein the first and second lox sites are loxP.

132. The method of claim 131, wherein the Cre mutant polypeptide is the polypeptide of claim 1.

133. The method of claim 130, wherein the organism is a prokaryotic or eukaryotic.

134. The method of claim 130, wherein the organism is selected from the group consisting of a bacteria, a mammal and a plant.

135. A method for producing selective site-specific recombination of a first nucleotide sequence having a first target DNA segment and a second nucleotide sequence having a second target DNA segment, the method comprising:
(a) introducing into the first nucleotide sequence a first lox site and a second lox site such that the lox sites flank the first target DNA segment, each of the first and second lox sites having from one to three additional nucleotides in the spacer region;
(b) introducing into the second nucleotide sequence a third lox site and a fourth lox site such that the lox sites flank the second target DNA segment;
(c) contacting the first nucleic acid sequence with a Cre mutant polypeptide that can catalyze site specific recombination at a lox site having from one to three additional, thereby producing site specific recombination; and
(d) contacting the second nucleic acid sequence with a Cre polypeptide that can catalyze site specific recombination at wild-type lox sites but not at lox sites having from one to three additional nucleotides in the spacer region, thereby producing site specific recombination.

136. The method of claim 135, wherein the site specific recombination occurs within a cell of an organism that is prokaryotic or eukaryotic.

137. The method of claim 136, wherein the cell is selected from the group consisting of bacterial, mammalian and plant.

138. A Cre/lox system comprising:
(a) a purified mutant Cre polypeptide that can catalyze site specific recombination at a lox site having from one to three additional nucleotides in the spacer region; and
(b) an isolated lox nucleotide sequence with from one to three additional nucleotides in the spacer region.

139. A Cre/lox system comprising
(a) a purified mutant Cre polypeptide that can catalyze site specific recombination at a lox site having from one to three additional nucleotides in the spacer region;
(b) an isolated lox nucleotide sequence with from one to three additional nucleotides in the spacer region;
(c) a purified Cre polypeptide that can catalyze site specific recombination at wild-type lox sites but not at lox sites having from one to three additional nucleotides in the spacer region; and
(d) an isolated wild-type lox nucleotide sequence.

140. A kit for producing site-specific recombination of a nucleotide sequence, the kit comprising:
(a) a purified mutant Cre polypeptide that can catalyze site specific recombination at a lox site having from one to three additional nucleotides in the spacer region;
(b) an isolated lox nucleotide sequence with from one to three additional nucleotides in the spacer region; and
(c) instructions for producing site specific recombination of the nucleotide sequence.

141. A kit for producing selective site-specific recombination of a nucleotide sequence, the kit comprising:
(a) a purified mutant Cre polypeptide that can catalyze site specific recombination at a lox site having from one to three additional nucleotides in the spacer region;
(b) an isolated lox nucleotide sequence with from one to three additional nucleotides in the spacer region;
(c) a purified Cre polypeptide that can catalyze site specific recombination at wild-type lox sites but not at lox sites having from one to three additional nucleotides in the spacer region;
(d) an isolated wild-type lox nucleotide sequence; and
(e) instructions for producing selective site specific recombination of the nucleotide sequence.

142. A cell comprising at least two mutant lox sites of claim 92 and the Cre mutant polypeptide of claim 1.

143. The cell of claim 142, wherein the cell is a prokaryotic or eukaryotic cell.

144. The cell of claim 142, wherein the cell is a bacterial cell.

145. The cell of claim 142, wherein the cell is a mammalian cell.

146. The cell of claim 142, wherein the cell is a plant cell.

147. A nucleic acid sequence comprising a lox site of claim 92.


SEQUENCE LISTING (390 sequences)

SEQ ID NO: 1 - protein, 343 aa, Enterobacteria phage P1
MSNLLTVHQNLPALPVDATSDEVRKNLMDMFRDRQAFSEHTWKMLLSVCRSWAAWCKLNNRKWFPAEPEDVRDYLLYLQARGLAVKTIQQHLGQLNMLHRRSGLPRPSDSNAVSLVMRRIRKENVDAGERAKQALAFERTDFDQVRSLMENSDRCQDIRNLAFLGIAYNTLLRIAEIARIRVKDISRTDGGRMLIHIGRTKTLVSTAGVEKALSLGVTKLVERWISVSGVADDPNNYLFCRVRKNGVAAPSATSQLSTRALEGIFEATHRLIYGAKDDSGQRYLAWSGHSARVGAARDMARAGVSIPEIMQAGGWTNVNIVMNYIRNLDSETGAMVRLLEDGD

SEQ ID NO: 2 - protein, 348 aa, Enterobacteria phage P1
MSNLLTVHQNLPALLVDACGRNATSDEVRKNLMDMFRDRQAFSEHTWKMLLSVCRSWAAWCKLNNRKWFPAEPEDVRDYLLYLQARGLAVKTIQQHLGQLNMLHRRSGLPRPSDSNAVSLVMRRIRKENVDAGERAKQALAFERTDFDQVRSLMENSDRCQDIRNLAFLGIAYNTLLRIAEIARIRVKDISRTDGGRMLIHIGRTKTLVSTAGVEKALSLGVTKLVERWISVSGVADDPNNYLFCRVRKNGVAAPSATSQLSTRALEGIFEATHRLIYGAKDDSGQRYLAWSGHSARVGAARDMARAGVSIPEIMQAGGWTNVNIVMNYIRNLDSETGAMVRLLEDGD

SEQ ID NO: 3 - protein, 348 aa, Enterobacteria phage P1
MSNLLTVHQNLPALPVDATSDEVRCGRIRKNLMDMFRDRQAFSEHTWKMLLSVCRSWAAWCKLNNRKWFPAEPEDVRDYLLYLQARGLAVKTIQQHLGQLNMLHRRSGLPRPSDSNAVSLVMRRIRKENVDAGERAKQALAFERTDFDQVRSLMENSDRCQDIRNLAFLGIAYNTLLRIAEIARIRVKDISRTDGGRMLIHIGRTKTLVSTAGVEKALSLGVTKLVERWISVSGVADDPNNYLFCRVRKNGVAAPSATSQLSTRALEGIFEATHRLIYGAKDDSGQRYLAWSGHSARVGAARDMARAGVSIPEIMQAGGWTNVNIVMNYIRNLDSETGAMVRLLEDGD

SEQ ID NO: 4 - protein, 348 aa, Enterobacteria phage P1
MSNLLTVHQNLPALPVDATSDEVRKNLMDMFRDRQAFSEHTWKMLLSVCRSWAAWCKLNNRKWFPAEPEDVRDYLLYLQARGLAVKTIQQHLGQLNMLHRRSGLPRPSDSNAVSLVMRRIRKENVDAGERAKQALAFERTDFDQVRSLMENSDRCQDIRNLAFLGIAYNTLLRIAEIARIRVKDISRTDGGRMLIHIGRTKTLVSTAGVEKALSLGVTKLVERWISVSGVADDPNNYLFCRVRKNGVAAPSATSQLSTRALEGIFEATHRLIYGAKDDSGCGRTGQRYLAWSGHSARVGAARDMARAGVSIPEIMQAGGWTNVNIVMNYIRNLDSETGAMVRLLEDGD

SEQ ID NO: 5 - protein, 348 aa, Enterobacteria phage P1
MSNLLTVHQNLPALPVDATSDEVRKNLMDMFRDRQAFSEHTWKMLLSVCRSWAAWCKLNNRKWFPAEPEDVRDYLLYLQARGLAVKTIQQHLGQLNMLHRRSGLPRPSDSNAVSLVMRRIRKENVDAGERAKQALAFERTDFDQVRSLMENSDRCQDIRNLAFLGIAYNTLLRIAEIARIRVKDISRTDGGRMLIHIGRTKTLVSTAGVEKALSLGVTKLVERWISVSGVADDPNNYLFCRVRKNGVAAPSATSQLSTRALEGIFEATHRLIYGAKDDSGQRYLAWLRPHWSGHSARVGAARDMARAGVSIPEIMQAGGWTNVNIVMNYIRNLDSETGAMVRLLEDGD

SEQ ID NO: 6 - protein, 355 aa, Pseudomonas sp. ADP
MLVVIPLRAPVFGDVAMSTTPADPDSVALLETRWASTLRNIVTPKEHLELAERHRAFLAAATSKNTRATYRSAIKHFLDWGGVLPAEEADVIRYLVRFADQHTSRTLALRLTALSQWHAYQCFPDPAGGATVRKTLAGIARTHGRPKRKAKALPVEDLERIAAALVGAGTLKSARDNALLQVGFFGGFRRGELAGIEVDHLDWDARGLVITLPRSKTDQEGEGIVKAIPYGDGPCCPTRALRTWLDAAGIAGGPVFRSITKWGVVGADALNPASVNAILADAARLAGLGYVPELSSHSLRRGMATSAHRAGADFRDIKKQGGWRHDGTVQGYIEEAEIFESNAAGSLLRSRVKPG

SEQ ID NO: 7 - protein, 379 aa, Pseudomonas pavonaceae
MLVEAPTVGNSRQSEPVVSADVRARIARSVAESKSPSTVRAYASDWRRFDTWCALHGHQELPADPLVVAAYLTDAADTLTDTGHRAYAPATLSRWVAAIGHRHQVAGYPPPTTDPIVTATLSGIRRSYAAAGDRPRRQMAPLLTSDIVTIVTAAREAVTGWAGEVLERRDTALLLMGFAGAFRRSELVGLDCGDIAVHRLDGLHVRLRRSKTDQDGLGVVKALPFTASHVSCPPCAVLRWLQVVAEYERGGRAGVIRLLRTAPGFDGHLCRGAVPTASPNTPLFRSIAKNGNLSTTALSGAAVHAAVRRRAAAAGYDETLVARLGGHSLRAGFVTQAFRNGADAHAIMRQTGHKTPGMLEVYAREHAPLIGNAVTDIGL

SEQ ID NO: 8 - protein, 371 aa, Streptomyces coelicolor
MGETGRQLAVVTADADVVEAELVDDETAGASVVVHTDRDRHLSPETVAAIAASVADSTRRAYGTDRAAFAAWCAEEDRTAVPASAETMAEWVRHLTVTPRPRTQRPAGPSTIERAMSAVTTWHEEQGRPKPNMRGARAVLNAYKDRLAVEKAEAAQARQATAALPPQIRAMLAGVDRTTLAGKRNAALVLLGFATAARVSELVALDVDTVTEAEHGYDVTLYRKKVRKHTPNPILYGTDPATCPVRALRAYLAALAAAGRTDGPLFVRVDRWDRLAPPMTRRGRVIGDPAGRMTAEAAAEVIERLAVAAGLSGDWSGHSLRRGFATAARAAGHDPLEIARAGGWVDGSRVLARYMDDVDRVKNSPLVGIGL

SEQ ID NO: 9 - protein, 389 aa, Mesorhizobium loti MAFF303099
MMDRKAEGEHTAGEDKASDDLPDIVDVVMEMGQAPTDPPSPPPQPAYRSQPASSSEPTLGLPAHLERLADHARKYVQAASSANTRRAYAADWKHFAAWCRRQHLDPLPPDPQIVGLYITACASGKGTGDKKPNSVSTIERRLSSLTWNFSQRGQPLDRKDRHIATVLAGIRNSHASPPRQKEAILPEDLIAMLETLDRGALRGLRDRGMLLLGFAGGLRRSEIVGLDCGRDQTEDGRGWIEILDKGILVTLRGKTGWREVEIGRGSSDTTCPVVALQTWLKLARIAHGPLFRRVTGQGKAIGSERLNDQEVARLVKRAALAAGVRGDLSEGERATKFSGHSLRAGLASSAEVDERYVQKQLGHTTAEMTRRYQRRRDRFRVNLTKASGL

SEQ ID NO: 10 - protein, 369 aa, Shewanella oneidensis MR-1
MSKSIQIYTADDSHSHQAVGISANLTKPFTQGDKTFFEESSLPQSVHADFYNAASETEYEISNNTRRVYRISFSFFEQYCLEHNLQSLPADPRSIISFIGHQKELLQASTGMQLSKQTLTTRIAAIRFYHIQAGFPTPTEHPQVIRVMRGLSRNHHRLVQDYDQQPIMYDEVELLIQAVDQQPHPLLRLRDKAIIQLGLQGGFRRSELANLKVHYLSFMRDKLKVRLPFSKSNQQGLREWKSLPDSEPFAAYHAVKSWLNESQITDGHLFRSISRDGKTLRPYHVNDNSKPKSTFSRNSGFLNGDDIYRIIKQYCLKAGLPAQYYGAHSLRSGCVTQLHENNKDILYIMARTGHTDPRSLRHYLKPKED

SEQ ID NO: 11 - protein, 318 aa, Leptospira interrogans serovar
MILWRISLGDYPFQFPEFSSESLNETAKKFINYLKIEKNYSQNTINAYSIDLKFFFEFCEKEQLDIFQIEPVDIRSYFAYLAKKHEIDRRSQSRKLSSLRTFYKVLLREDLVKSNPATQLSFPKVRKEVPKNFRINETEEILEFESENASEVSEIRDRAMIEVLYSSGLRVFELVNAKLNSLSKDLTVLKVLGKGRKERFVYFGKEAVSSLQKYLEYRNVSFPDAEEIFLNQRGKKLTTRGVRYILNERRKKMGWEKTITPHKFRHTFATDLLDAGAEIRAVQELLGHSSLSTTQIYLSVSKEKIKEVYRKAHPHARK

SEQ ID NO: 12 - protein, 341 aa, Selenomonas ruminantium
MLLYILLIESRFIMKIKDNFMLIKNARIENNERLSLKAKRRLEKSKADNTLKAYACDWSDFSDWCQYHGVTDLPASPETIVNYINDLADDAKANTVSRRVTAISENHIAAGFSGRHNPAKDGMVRAAMSAIRREKGTFQRGKSPILMETLYLLADLFDEEKLSGLRDKALIYLGFAGAFRRSELVHIQYEDLTFTPQGVIIFMAHSKGDQLGHGEQIAIPYAPQAEICAVRALKKWLDTAQIHRGPIFRPITRVQSLRNTQLSDKSVALIVKKYVGLAGLDEHLFAGHSLRRGFATSAAQHDIDALTIMRQTRHKSEKMVHRYIEQGNIFKDNALNRMYNK

SEQ ID NO: 13 - protein, 346 aa, Agrobacterium tumefaciens
MTDQDVETLRHLVNQGMGDNTLRALTSDLAYLEAWGLATTGSSLPWPAPEALLLKFVAHHLWDPEKRATDPDHGMPAAVDENLRRQGFLRSVGPHAPSTVRRRLANWSTLTRWRGLHGAFASPALKSAIRLAVRAVPRTRARKSAKAVTGDVLAKLLATCESDSLRDLRDKAILMVAFASGGRRRSEIAGLRREQLTIEAPIETEGGPPLPSLAIHLGRTKTTSGEEDDTVFLTGRPVEALNAWLAAAKIDKGSVFRGIGRWGTVSRRALDPQSVNAILKQRAEMAGLEAGQFSAHGLRSGYLTEAANRGIPLPEAMEQSRHRSVQQASSYYNSATRRSGRAARLL

SEQ ID NO: 14 - protein, 393 aa, Salmonella typhi
MNSKPVTRQFEDSDLHQELVTFEVPNNDLKELIFYFSHMKYNTAKTYLQWLRSWNEWYQANAGKEGNEAWPASSLPVTEPPLLAYLDYLQGSLSHSSIKGCLHALNSIHRKALDRPGIITSKVKSILASLEQAEAREQKVTRQATPFLVSDLKALIKAHGTTQSVRKLRDLCIIWTGFETLLRSAELRRIRMQDLVLNEQTGSFTLTVYRTKSTVSTLLTYHLTPHLTATLIRLMDMVKRDQQSHPKDYLFQAVNYQDSGYMPPGWGLRSKGNEINTLLKNHNMPYRPTRPPIGKNGKPIIVDDEGMLSKNTLLRAFEAFWDELHPQEAGTRCWTGHSVRVGGAIELANAGYTHLQIMEMGNWSNPEMVSRYIRNIDAGKKAMTKFMREALDE

SEQ ID NO: 15 - protein, 302 aa, Mesorhizobium loti
MPYPVLDAPISPLRQRLIDDMNMRRFSQETQRNYLRDIGRLATFLGRSPHTATTDDLRRFQIEQQDDGVPVPTMNSIVSALRFFFTHTVDRPDLARKLVRLAHPRKLPVVLSRDEVARLLNATTCLKHQAALSVAYGAGLRVAEVSALKVADIDSERMLIRVERGKGGRYRNAMLSQDLLLLLRQWWKVGRQQGVMHRDGWLFPGQHAMKPISTRQLYRVVVEAAQAADIAKRVGPHTLRHSFATHLLEDGTDIRIIQVLLGHAKLNSTAFYTKVATRTVRTVTSPLDKLGLFKPEELSPDG

SEQ ID NO: 16 - protein, 319 aa, Nostoc sp.
MREDTTRQIDLVLSTPLPLTLHPAAVYLSSLSPTSRRTMEKALNVIARLLTSNQCDAMSLDWSKLRYQHTAAIRAIFIEQYSPATTNRMLCAMRRVLKESLRLGFMSAQDYQYAIDLKSVRGDSGLPGRLIKPEEITSLLRNCLQDNVIGIRDAALIGILSSCGLRRSEAVALEMNDFNREDNLLTVRQGKGGKSRRVYLPPGVVGILNDWLKIRGKSSGALICPVKRGGHIHIQHLTDQAVMAICQKRADSTGIKPFSPHDFRRTFVTRLLESGIDVLTVSQLAGHVNLATTQKYDLRGEAAKRKAVECLNFLYENFF

SEQ ID NO: 17 - protein, 335 aa, Magnetococus sp.
MRIQAMHKMENPFGDGHCLIANDLNKIDQLLRQDGVAACPADRTHKARSSDAKRFVQWCQQQGVKALPASPETVTGYIEAMIQDKALATVRRYVSSISTLHSAVEMCNPAHSPEVRESLRKAADQCERPSKGTRPITREMVQRMVQATLGSTRDLRDVALLMVAYDTMLRRTEMVALDVANFHFGRDGFATVTCHSEDDLALPTTRCIAPDTVRAVEAWMRASNTSTGPMFRSIDRAGVIGDRLSDRGLVRAFKRLARQAGLDPEGISGLSCRVGAAGDMMKEGFRLKEVMQAGGWRSPVMVSRYHQQKRALADNEDLAESPLTRVMLHKPGKRA

SEQ ID NO: 18 - DNA, 34 bp, Enterobacteria phage P1
ataacttcgtataatgtatgctatacgaagttat

SEQ ID NO: 19 - DNA, 11 bp, Artificial Sequence
  Mutant lox sites
aagaacaagaa

SEQ ID NO: 20 - DNA, 11 bp, Artificial Sequence
  Mutant lox sites
aaacaacaaga

SEQ ID NO: 21 - DNA, 11 bp, Artificial Sequence
  Mutant lox sites
agaaagaaaga

SEQ ID NO: 22 - DNA, 11 bp, Artificial Sequence
  Mutant lox sites
aaaaaaacgca

SEQ ID NO: 23 - DNA, 11 bp, Artificial Sequence
  Mutant lox sites
aggcaaaaaaa

SEQ ID NO: 24 - DNA, 11 bp, Artificial Sequence
  Mutant lox sites
caaaaaaaagc

SEQ ID NO: 25 - DNA, 11 bp, Artificial Sequence
  Mutant lox sites
aagaaaaaacc

SEQ ID NO: 26 - DNA, 11 bp, Artificial Sequence
  Mutant lox sites
caaaaaacgaa

SEQ ID NO: 27 - DNA, 11 bp, Artificial Sequence
  Mutant lox sites
gaaaaaaaacg

SEQ ID NO: 28 - DNA, 11 bp, Artificial Sequence
  Mutant lox sites
cggaaaaaaaa

SEQ ID NO: 29 - DNA, 11 bp, Artificial Sequence
  Mutant lox sites
attatgatcat

SEQ ID NO: 30 - DNA, 11 bp, Artificial Sequence
  Mutant lox sites
aaatttggaaa

SEQ ID NO: 31 - DNA, 11 bp, Artificial Sequence
  Mutant lox sites
tatatatatgc

SEQ ID NO: 32 - DNA, 11 bp, Artificial Sequence
  Mutant lox sites
tttcaaacttt

SEQ ID NO: 33 - DNA, 11 bp, Artificial Sequence
  Mutant lox sites
cgattattatt

SEQ ID NO: 34 - DNA, 11 bp, Artificial Sequence
  Mutant lox sites
aaagacaaaaa

SEQ ID NO: 35 - DNA, 11 bp, Artificial Sequence
  Mutant lox sites
tttctgttttt

SEQ ID NO: 36 - DNA, 11 bp, Artificial Sequence
  Mutant lox sites
gcatatatata

SEQ ID NO: 37 - DNA, 11 bp, Artificial Sequence
  Mutant lox sites
tttttaaaacc

SEQ ID NO: 38 - DNA, 11 bp, Artificial Sequence
  Mutant lox sites
aaaagcttttt

SEQ ID NO: 39 - DNA, 11 bp, Artificial Sequence
  Mutant lox sites
aaaaaaaaaag

SEQ ID NO: 40 - DNA, 11 bp, Artificial Sequence
  Mutant lox sites
catatatatat

SEQ ID NO: 41 - DNA, 11 bp, Artificial Sequence
  Mutant lox sites
ttttttttttg

SEQ ID NO: 42 - DNA, 11 bp, Artificial Sequence
  Mutant lox sites
attattattac

SEQ ID NO: 43 - DNA, 11 bp, Artificial Sequence
  Mutant lox sites
taataatattg

SEQ ID NO: 44 - DNA, 11 bp, Artificial Sequence
  Mutant lox sites
gaaaaattttt

SEQ ID NO: 45 - DNA, 11 bp, Artificial Sequence
  Mutant lox sites
attttcaaaaa

SEQ ID NO: 46 - DNA, 11 bp, Artificial Sequence
  Mutant lox sites
tgaaaatttaa

SEQ ID NO: 47 - DNA, 11 bp, Artificial Sequence
  Mutant lox sites
aattaatctaa

SEQ ID NO: 48 - DNA, 11 bp, Artificial Sequence
  Mutant lox sites
ttagattaata

SEQ ID NO: 49 - DNA, 11 bp, Artificial Sequence
  Mutant lox sites
aaaaaaaaaaa

SEQ ID NO: 50 - DNA, 11 bp, Artificial Sequence
  Mutant lox sites
ttttttttttt

SEQ ID NO: 51 - DNA, 11 bp, Artificial Sequence
  Mutant lox sites
tatatatatat

SEQ ID NO: 52 - DNA, 11 bp, Artificial Sequence
  Mutant lox sites
atatatatata

SEQ ID NO: 53 - DNA, 11 bp, Artificial Sequence
  Mutant lox sites
atttatttatt

SEQ ID NO: 54 - DNA, 11 bp, Artificial Sequence
  Mutant lox sites
taataataata

SEQ ID NO: 55 - DNA, 11 bp, Artificial Sequence
  Mutant lox sites
attaattaatt

SEQ ID NO: 56 - DNA, 11 bp, Artificial Sequence
  Mutant lox sites
taattaattaa

SEQ ID NO: 57 - DNA, 11 bp, Artificial Sequence
  Mutant lox sites
ataaaatttta

SEQ ID NO: 58 - DNA, 11 bp, Artificial Sequence
  Mutant lox sites
tattttaaaat

SEQ ID NO: 59 - DNA, 11 bp, Artificial Sequence
  Mutant lox sites
atgtattttta

SEQ ID NO: 60 - DNA, 11 bp, Artificial Sequence
  Mutant lox sites
tgtataaaaat

SEQ ID NO: 61 - DNA, 11 bp, Artificial Sequence
  Mutant lox sites
attgtatgtta

SEQ ID NO: 62 - DNA, 11 bp, Artificial Sequence
  Mutant lox sites
tatgcatatat

SEQ ID NO: 63 - DNA, 11 bp, Artificial Sequence
  Mutant lox sites
aataattatgc

SEQ ID NO: 64 - DNA, 11 bp, Artificial Sequence
  Mutant lox sites
tttatgtaaaa

SEQ ID NO: 65 - DNA, 11 bp, Artificial Sequence
  Mutant lox sites
atatgtatata

SEQ ID NO: 66 - DNA, 11 bp, Artificial Sequence
  Mutant lox sites
attaatgtatg

SEQ ID NO: 67 - DNA, 11 bp, Artificial Sequence
  Mutant lox sites
gtatgaaatta

SEQ ID NO: 68 - DNA, 11 bp, Artificial Sequence
  Mutant lox sites
aataatgtatt

SEQ ID NO: 69 - DNA, 11 bp, Artificial Sequence
  Mutant lox sites
aaaaatgtatt

SEQ ID NO: 70 - DNA, 11 bp, Artificial Sequence
  Mutant lox sites
tatgtatgtaa

SEQ ID NO: 71 - DNA, 11 bp, Artificial Sequence
  Mutant lox sites
tgtatgctaat

SEQ ID NO: 72 - DNA, 11 bp, Artificial Sequence
  Mutant lox sites
tttatgtataa

SEQ ID NO: 73 - DNA, 11 bp, Artificial Sequence
  Mutant lox sites
atatgtatata

SEQ ID NO: 74 - DNA, 11 bp, Artificial Sequence
  Mutant lox sites
tatgtatgcta

SEQ ID NO: 75 - DNA, 11 bp, Artificial Sequence
  Mutant lox sites
aattgtatgct

SEQ ID NO: 76 - DNA, 11 bp, Artificial Sequence
  Mutant lox sites
tttatgtatga

SEQ ID NO: 77 - DNA, 11 bp, Artificial Sequence
  Mutant lox sites
aaaaaatgtat

SEQ ID NO: 78 - DNA, 11 bp, Artificial Sequence
  Mutant lox sites
atgtatattat

SEQ ID NO: 79 - DNA, 11 bp, Artificial Sequence
  Mutant lox sites
tttgtatgctt

SEQ ID NO: 80 - DNA, 11 bp, Artificial Sequence
  Mutant lox sites
aaatgtatgca

SEQ ID NO: 81 - DNA, 11 bp, Artificial Sequence
  Mutant lox sites
atattgtatgc

SEQ ID NO: 82 - DNA, 11 bp, Artificial Sequence
  Mutant lox sites
tgtatgcaatt

SEQ ID NO: 83 - DNA, 11 bp, Artificial Sequence
  Mutant lox sites
atgtatgttaa

SEQ ID NO: 84 - DNA, 11 bp, Artificial Sequence
  Mutant lox sites
tatgtatgtaa

SEQ ID NO: 85 - DNA, 11 bp, Artificial Sequence
  Mutant lox sites
aaatgtatgat

SEQ ID NO: 86 - DNA, 11 bp, Artificial Sequence
  Mutant lox sites
ttaatgtatgt

SEQ ID NO: 87 - DNA, 11 bp, Artificial Sequence
  Mutant lox sites
atatatgtatg

SEQ ID NO: 88 - DNA, 11 bp, Artificial Sequence
  Mutant lox sites
tatgtatgcat

SEQ ID NO: 89 - DNA, 11 bp, Artificial Sequence
  Mutant lox sites
atgcatgtatt

SEQ ID NO: 90 - DNA, 11 bp, Artificial Sequence
  Mutant lox sites
gtatgcataaa

SEQ ID NO: 91 - DNA, 11 bp, Artificial Sequence
  Mutant lox sites
ttacgtatgta

SEQ ID NO: 92 - DNA, 11 bp, Artificial Sequence
  Mutant lox sites
atatgcatgat

SEQ ID NO: 93 - DNA, 11 bp, Artificial Sequence
  Mutant lox sites
tttgtatgcat

SEQ ID NO: 94 - DNA, 11 bp, Artificial Sequence
  Mutant lox sites
aaatgtatgca

SEQ ID NO: 95 - DNA, 11 bp, Artificial Sequence
  Mutant lox sites
ttgtatgcaaa

SEQ ID NO: 96 - DNA, 11 bp, Artificial Sequence
  Mutant lox sites
tatatgtatgc

SEQ ID NO: 97 - DNA, 11 bp, Artificial Sequence
  Mutant lox sites
acgtatgtata

SEQ ID NO: 98 - DNA, 11 bp, Artificial Sequence
  Mutant lox sites
cgtatgtaata

SEQ ID NO: 99 - DNA, 11 bp, Artificial Sequence
  Mutant lox sites
agtatgtatgc

SEQ ID NO: 100 - DNA, 11 bp, Artificial Sequence
  Mutant lox sites
atgtatgcgat

SEQ ID NO: 101 - DNA, 11 bp, Artificial Sequence
  Mutant lox sites
aatgttatggc

SEQ ID NO: 102 - DNA, 11 bp, Artificial Sequence
  Mutant lox sites
atgtgatatgc

SEQ ID NO: 103 - DNA, 11 bp, Artificial Sequence
  Mutant lox sites
gcgtatgtata

SEQ ID NO: 104 - DNA, 11 bp, Artificial Sequence
  Mutant lox sites
cgtatgtagta

SEQ ID NO: 105 - DNA, 11 bp, Artificial Sequence
  Mutant lox sites
gtattggcagt

SEQ ID NO: 106 - DNA, 11 bp, Artificial Sequence
  Mutant lox sites
tatgcatgtag

SEQ ID NO: 107 - DNA, 11 bp, Artificial Sequence
  Mutant lox sites
tgtaagttagc

SEQ ID NO: 108 - DNA, 11 bp, Artificial Sequence
  Mutant lox sites
ttagcatgtag

SEQ ID NO: 109 - DNA, 11 bp, Artificial Sequence
  Mutant lox sites
tcaatgtatgc

SEQ ID NO: 110 - DNA, 11 bp, Artificial Sequence
  Mutant lox sites
cgtatgtatca

SEQ ID NO: 111 - DNA, 11 bp, Artificial Sequence
  Mutant lox sites
atgctactgta

SEQ ID NO: 112 - DNA, 11 bp, Artificial Sequence
  Mutant lox sites
tgtaacttgct

SEQ ID NO: 113 - DNA, 11 bp, Artificial Sequence
  Mutant lox sites
gtaatgccatt

SEQ ID NO: 114 - DNA, 11 bp, Artificial Sequence
  Mutant lox sites
attagcatgtc

SEQ ID NO: 115 - DNA, 11 bp, Artificial Sequence
  Mutant lox sites
catcgtatgta

SEQ ID NO: 116 - DNA, 11 bp, Artificial Sequence
  Mutant lox sites
atgttaactgc

SEQ ID NO: 117 - DNA, 11 bp, Artificial Sequence
  Mutant lox sites
attgtattgcc

SEQ ID NO: 118 - DNA, 11 bp, Artificial Sequence
  Mutant lox sites
gcatctagtat

SEQ ID NO: 119 - DNA, 11 bp, Artificial Sequence
  Mutant lox sites
tatatgtatgc

SEQ ID NO: 120 - DNA, 11 bp, Artificial Sequence
  Mutant lox sites
cgtatgtaatt

SEQ ID NO: 121 - DNA, 11 bp, Artificial Sequence
  Mutant lox sites
atagttattgc

SEQ ID NO: 122 - DNA, 11 bp, Artificial Sequence
  Mutant lox sites
ttgtaatgcat

SEQ ID NO: 123 - DNA, 11 bp, Artificial Sequence
  Mutant lox sites
atgttatatgc

SEQ ID NO: 124 - DNA, 11 bp, Artificial Sequence
  Mutant lox sites
attatgcatgt

SEQ ID NO: 125 - DNA, 11 bp, Artificial Sequence
  Mutant lox sites
gtatgcattat

SEQ ID NO: 126 - DNA, 11 bp, Artificial Sequence
  Mutant lox sites
atgtcaattgt

SEQ ID NO: 127 - DNA, 11 bp, Artificial Sequence
  Mutant lox sites
aatgttattgc

SEQ ID NO: 128 - DNA, 11 bp, Artificial Sequence
  Mutant lox sites
catgttatatg

SEQ ID NO: 129 - DNA, 11 bp, Artificial Sequence
  Mutant lox sites
taaatgtatgc

SEQ ID NO: 130 - DNA, 11 bp, Artificial Sequence
  Mutant lox sites
atgtatgctaa

SEQ ID NO: 131 - DNA, 11 bp, Artificial Sequence
  Mutant lox sites
cgtaaattgta

SEQ ID NO: 132 - DNA, 11 bp, Artificial Sequence
  Mutant lox sites
tgcatagtata

SEQ ID NO: 133 - DNA, 11 bp, Artificial Sequence
  Mutant lox sites
aatgttatagc

SEQ ID NO: 134 - DNA, 11 bp, Artificial Sequence
  Mutant lox sites
tatagctatag

SEQ ID NO: 135 - DNA, 11 bp, Artificial Sequence
  Mutant lox sites
aatcgtatgta

SEQ ID NO: 136 - DNA, 11 bp, Artificial Sequence
  Mutant lox sites
aatcgtatgta

SEQ ID NO: 137 - DNA, 11 bp, Artificial Sequence
  Mutant lox sites
attgcaatgat

SEQ ID NO: 138 - DNA, 11 bp, Artificial Sequence
  Mutant lox sites
tgtaatatgca

SEQ ID NO: 139 - DNA, 34 bp, Enterobacteria phage P1
tattgaagcatattacatacgatatgcttcaata

SEQ ID NO: 140 - protein, 298 aa, Escherichia coli
MKQDLARIEQFLDALWLEKNLAENTLNAYRRDLSMMVEWLHHRGLTLATAQSDDLQALLAERLEGGYKATSSARLLSAVRRLFQYLYREKFREDDPSAHLASPKLPQRLPKDLSEAQVERLLQAPLIDQPLELRDKAMLEVLYATGLRVSELVGLTMSDISLRQGVVRVIGKGNKERLVPLGEEAVYWLETYLEHGRPWLLNGVSIDVLFPSQRAQQMTRQTFWHRIKHYAVLAGIDSEKLSPHVLRHAFATHLLNHGADLRVVQMLLGHSDLSTTQIYTHVATERLRQLHQQHHPRA

SEQ ID NO: 141 - DNA, 52 bp, Enterobacteria phage P1
tcgagtgcacaacttcgtataatgtatgctatacgaagttatcattcgctag

SEQ ID NO: 142 - DNA, 55 bp, Enterobacteria phage P1
tcgagtgcacaacttcgtataatgatttatgctatacgaagttatcattcgctag

SEQ ID NO: 143 - DNA, 19 bp, Enterobacteria phage P1
gtgcacaacttcgtataat

SEQ ID NO: 144 - DNA, 29 bp, Enterobacteria phage P1
gtatgctatacgaagttatcattcgctag

SEQ ID NO: 145 - DNA, 32 bp, Enterobacteria phage P1
gatttatgctatacgaagttatcattcgctag

SEQ ID NO: 146 - protein, 348 aa, Enterobacteria phage P1
MSNLLTVHQNLPALPVDATSDEVRKNLMDMFRDRQAFSEHTWKMLLSVCRSWAAWCKLNNRKWFPAEPEDVRDYLLYLQARGLAVKTIQQHLGQLNMLHRRSGLPRPSDSNAVSLVMRRIRKENVDAGERAKQALAFERTDFDQVRSLMENSDRCQDIRNLAFLGIAYNTLLRIAEIARIRVKDISRTDGGRMLIHIGRTKTLVSTAGVEKALSLGVTKLVERWISVSGVADDPNNYLFCRVRKNGVAAPSATSQLSTRALEGIFEATHRLIYGAKDDCGRNDSGQRYLAWSGHSARVGAARDMARAGVSIPEIMQAGGWTNVNIVMNYIRNLDSETGAMVRLLEDGD

SEQ ID NO: 147 - protein, 348 aa, Enterobacteria phage P1
MSNLLTVHQNLPALPVDATSDEVRKNLMDMFRDRQAFSEHTWKMLLSVCRSWAAWCKLNNRKWFPAEPEDVRDYLLYLQARGLAVKTIQQHLGQLNMLHRRSGLPRPSDSNAVSLVMRRIRKENVDAGERAKQALAFERTDFDQVRSLMENSDRCQDIRNLAFLGIAYNTLLRIAEIARIRVKDISRTDGGRMLIHIGRTKTLVSTAGVEKALSLGVTKLVERWISVSGVADDPNNYLFCRVRKNGVAAPSATSQLSTRALEGIFEATHRLIYGAKDDSVRPHSGQRYLAWSGHSARVGAARDMARAGVSIPEIMQAGGWTNVNIVMNYIRNLDSETGAMVRLLEDGD

SEQ ID NO: 148 - protein, 348 aa, Enterobacteria phage P1
MSNLLTVHQNLPALPVDATSDEVRKNLMDMFRDRQAFSEHTWKMLLSVCRSWAAWCKLNNRKWFPAEPEDVRDYLLYLQARGLAVKTIQQHLGQLNMLHRRSGLPRPSDSNAVSLVMRRIRKENVDAGERAKQALAFERTDFDQVRSLMENSDRCQDIRNLAFLGIAYNTLLRIAEIARIRVKDISRTDGGRMLIHIGRTKTLVSTAGVEKALSLGVTKLVERWISVSGVADDPNNYLFCRVRKNGVAAPSATSQLSTRALEGIFEATHRLIYGAKDDSGAAASGQRYLAWSGHSARVGAARDMARAGVSIPEIMQAGGWTNVNIVMNYIRNLDSETGAMVRLLEDGD

SEQ ID NO: 149 - protein, 348 aa, Enterobacteria phage P1
MSNLLTVHQNLPALPVDACGRNATSDEVRKNLMDMFRDRQAFSEHTWKMLLSVCRSWAAWCKLNNRKWFPAEPEDVRDYLLYLQARGLAVKTIQQHLGQLNMLHRRSGLPRPSDSNAVSLVMRRIRKENVDAGERAKQALAFERTDFDQVRSLMENSDRCQDIRNLAFLGIAYNTLLRIAEIARIRVKDISRTDGGRMLIHIGRTKTLVSTAGVEKALSLGVTKLVERWISVSGVADDPNNYLFCRVRKNGVAAPSATSQLSTRALEGIFEATHRLIYGAKDDSGQRYLAWSGHSARVGAARDMARAGVSIPEIMQAGGWTNVNIVMNYIRNLDSETGAMVRLLEDGD

SEQ ID NO: 150 - protein, 343 aa, Enterobacteria phage P1
MSNLLTVHQNLPALLVDATSDEVRKNLMDMFRDRQAFSEHTWKMLLSVCRSWAAWCKLNNRKWFPAEPEDVRDYLLYLQARGLAVKTIQQHLGQLNMLHRRSGLPRPSDSNAVSLVMRRIRKENVDAGERAKQALAFERTDFDQVRSLMENSDRCQDIRNLAFLGIAYNTLLRIAEIARIRVKDISRTDGGRMLIHIGRTKTLVSTAGVEKALSLGVTKLVERWISVSGVADDPNNYLFCRVRKNGVAAPSATSQLSTRALEGIFEATHRLIYGAKDDSGQRYLAWSGHSARVGAARDMARAGVSIPEIMQAGGWTNVNIVMNYIRNLDSETGAMVRLLEDGD

SEQ ID NO: 151 - DNA, 10 bp, Artificial Sequence
  Mutant lox sites
taactatgac

SEQ ID NO: 152 - DNA, 10 bp, Artificial Sequence
  Mutant lox sites
aatgatactg

SEQ ID NO: 153 - DNA, 10 bp, Artificial Sequence
  Mutant lox sites
ggattataac

SEQ ID NO: 154 - DNA, 10 bp, Artificial Sequence
  Mutant lox sites
tacactgtta

SEQ ID NO: 155 - DNA, 10 bp, Artificial Sequence
  Mutant lox sites
aaagcgtttt

SEQ ID NO: 156 - DNA, 10 bp, Artificial Sequence
  Mutant lox sites
tttgggaaaa

SEQ ID NO: 157 - DNA, 10 bp, Artificial Sequence
  Mutant lox sites
catatatacc

SEQ ID NO: 158 - DNA, 10 bp, Artificial Sequence
  Mutant lox sites
gctaattaac

SEQ ID NO: 159 - DNA, 10 bp, Artificial Sequence
  Mutant lox sites
tcgaattatc

SEQ ID NO: 160 - DNA, 10 bp, Artificial Sequence
  Mutant lox sites
ataggactta

SEQ ID NO: 161 - DNA, 10 bp, Artificial Sequence
  Mutant lox sites
aagtattgat

SEQ ID NO: 162 - DNA, 10 bp, Artificial Sequence
  Mutant lox sites
tcaatgatat

SEQ ID NO: 163 - DNA, 10 bp, Artificial Sequence
  Mutant lox sites
gtttataaag

SEQ ID NO: 164 - DNA, 10 bp, Artificial Sequence
  Mutant lox sites
gtttataaag

SEQ ID NO: 165 - DNA, 10 bp, Artificial Sequence
  Mutant lox sites
atatacctat

SEQ ID NO: 166 - DNA, 10 bp, Artificial Sequence
  Mutant lox sites
tatttggaat

SEQ ID NO: 167 - DNA, 10 bp, Artificial Sequence
  Mutant lox sites
aagaaattca

SEQ ID NO: 168 - DNA, 10 bp, Artificial Sequence
  Mutant lox sites
ttaacttctt

SEQ ID NO: 169 - DNA, 10 bp, Artificial Sequence
  Mutant lox sites
aatgaagata

SEQ ID NO: 170 - DNA, 10 bp, Artificial Sequence
  Mutant lox sites
tcttttatga

SEQ ID NO: 171 - DNA, 10 bp, Artificial Sequence
  Mutant lox sites
gatatatata

SEQ ID NO: 172 - DNA, 10 bp, Artificial Sequence
  Mutant lox sites
ctatatatat

SEQ ID NO: 173 - DNA, 10 bp, Artificial Sequence
  Mutant lox sites
tttttttttg

SEQ ID NO: 174 - DNA, 10 bp, Artificial Sequence
  Mutant lox sites
aaaaaaaaac

SEQ ID NO: 175 - DNA, 10 bp, Artificial Sequence
  Mutant lox sites
ttaatgtaat

SEQ ID NO: 176 - DNA, 10 bp, Artificial Sequence
  Mutant lox sites
aattacatta

SEQ ID NO: 177 - DNA, 10 bp, Artificial Sequence
  Mutant lox sites
ttgatttata

SEQ ID NO: 178 - DNA, 10 bp, Artificial Sequence
  Muant lox sites
aataatacat

SEQ ID NO: 179 - DNA, 10 bp, Artificial Sequence
  Mutant lox sites
tttagaatat

SEQ ID NO: 180 - DNA, 10 bp, Artificial Sequence
  Mutant lox sites
aaattttact

SEQ ID NO: 181 - DNA, 10 bp, Artificial Sequence
  Mutant lox sites
aaaaaaaaaa

SEQ ID NO: 182 - DNA, 10 bp, Artificial Sequence
  Mutant lox sites
tatatatata

SEQ ID NO: 183 - DNA, 10 bp, Artificial Sequence
  Mutant lox sites
atatatatat

SEQ ID NO: 184 - DNA, 10 bp, Artificial Sequence
  Mutant lox sites
aattaattaa

SEQ ID NO: 185 - DNA, 10 bp, Artificial Sequence
  Mutant lox sites
ttaattaatt

SEQ ID NO: 186 - DNA, 10 bp, Artificial Sequence
  Mutant lox sites
tttaaaaatt

SEQ ID NO: 187 - DNA, 10 bp, Artificial Sequence
  Mutant lox sites
tttttttttt

SEQ ID NO: 188 - DNA, 10 bp, Artificial Sequence
  Mutant lox sites
ttaattaaaa

SEQ ID NO: 189 - DNA, 10 bp, Artificial Sequence
  Mutant lox sites
aattttaaaa

SEQ ID NO: 190 - DNA, 10 bp, Artificial Sequence
  Mutant lox sites
aaataattta

SEQ ID NO: 191 - DNA, 10 bp, Artificial Sequence
  Mutant lox sites
atttgattaa

SEQ ID NO: 192 - DNA, 10 bp, Artificial Sequence
  Mutant lox sites
aagatatatg

SEQ ID NO: 193 - DNA, 10 bp, Artificial Sequence
  Mutant lox sites
cgttaattgt

SEQ ID NO: 194 - DNA, 10 bp, Artificial Sequence
  Mutant lox sites
tgtaagatct

SEQ ID NO: 195 - DNA, 10 bp, Artificial Sequence
  Mutant lox sites
acagtttaaa

SEQ ID NO: 196 - DNA, 10 bp, Artificial Sequence
  Mutant lox sites
ctgattaatg

SEQ ID NO: 197 - DNA, 10 bp, Artificial Sequence
  Mutant lox sites
ttaatatggc

SEQ ID NO: 198 - DNA, 10 bp, Artificial Sequence
  Mutant lox sites
tgcgtaattt

SEQ ID NO: 199 - DNA, 10 bp, Artificial Sequence
  Mutant lox sites
acaaaaatgg

SEQ ID NO: 200 - DNA, 10 bp, Artificial Sequence
  Mutant lox sites
caggtttttt

SEQ ID NO: 201 - DNA, 10 bp, Artificial Sequence
  Mutant lox sites
tagtatgcat

SEQ ID NO: 202 - DNA, 10 bp, Artificial Sequence
  Mutant lox sites
caagtatttg

SEQ ID NO: 203 - DNA, 10 bp, Artificial Sequence
  Mutant lox sites
atgttttacg

SEQ ID NO: 204 - DNA, 10 bp, Artificial Sequence
  Mutant lox sites
tatacgtagt

SEQ ID NO: 205 - DNA, 10 bp, Artificial Sequence
  Mutant lox sites
gtatgcaatt

SEQ ID NO: 206 - DNA, 10 bp, Artificial Sequence
  Mutant lox sites
tgttcatttg

SEQ ID NO: 207 - DNA, 10 bp, Artificial Sequence
  Mutant lox sites
cgaagaatta

SEQ ID NO: 208 - DNA, 10 bp, Artificial Sequence
  Mutant lox sites
aaagtagcat

SEQ ID NO: 209 - DNA, 10 bp, Artificial Sequence
  Mutant lox sites
tttatgtgca

SEQ ID NO: 210 - DNA, 10 bp, Artificial Sequence
  Mutant lox sites
atatatgcga

SEQ ID NO: 211 - DNA, 10 bp, Artificial Sequence
  Mutant lox sites
gtattatgca

SEQ ID NO: 212 - DNA, 10 bp, Artificial Sequence
  Mutant lox sites
atgcataatg

SEQ ID NO: 213 - DNA, 10 bp, Artificial Sequence
  Mutant lox sites
aaatgcgtaa

SEQ ID NO: 214 - DNA, 10 bp, Artificial Sequence
  Mutant lox sites
ttcgtatgtt

SEQ ID NO: 215 - DNA, 10 bp, Artificial Sequence
  Mutant lox sites
gatacatgat

SEQ ID NO: 216 - DNA, 10 bp, Artificial Sequence
  Mutant lox sites
cggatatatt

SEQ ID NO: 217 - DNA, 10 bp, Artificial Sequence
  Mutant lox sites
ttaaaagtgc

SEQ ID NO: 218 - DNA, 10 bp, Artificial Sequence
  Mutant lox sites
atgcgtttta

SEQ ID NO: 219 - DNA, 10 bp, Artificial Sequence
  Mutant lox sites
tattggatac

SEQ ID NO: 220 - DNA, 10 bp, Artificial Sequence
  Mutant lox sites
tgttattcga

SEQ ID NO: 221 - DNA, 10 bp, Artificial Sequence
  Mutant lox sites
aatgtatgct

SEQ ID NO: 222 - DNA, 10 bp, Artificial Sequence
  Mutant lox sites
atgctaatgt

SEQ ID NO: 223 - DNA, 10 bp, Artificial Sequence
  Mutant lox sites
gcatatttag

SEQ ID NO: 224 - DNA, 10 bp, Artificial Sequence
  Mutant lox sites
gaatgtatac

SEQ ID NO: 225 - DNA, 10 bp, Artificial Sequence
  Mutant lox sites
aattcgtatg

SEQ ID NO: 226 - DNA, 10 bp, Artificial Sequence
  Mutant lox sites
cttttagatg

SEQ ID NO: 227 - DNA, 10 bp, Artificial Sequence
  Mutant lox sites
ataacgagtt

SEQ ID NO: 228 - DNA, 10 bp, Artificial Sequence
  Mutant lox sites
tcgtatgtaa

SEQ ID NO: 229 - DNA, 10 bp, Artificial Sequence
  Mutant lox sites
atgagtttac

SEQ ID NO: 230 - DNA, 10 bp, Artificial Sequence
  Mutant lox sites
tgcattgtaa

SEQ ID NO: 231 - DNA, 10 bp, Artificial Sequence
  Mutant lox sites
aatgatatgc

SEQ ID NO: 232 - DNA, 10 bp, Artificial Sequence
  Mutant lox sites
cgtataagta

SEQ ID NO: 233 - DNA, 10 bp, Artificial Sequence
  Mutant lox sites
tatgcatgaa

SEQ ID NO: 234 - DNA, 10 bp, Artificial Sequence
  Mutant lox sites
gtaatagcat

SEQ ID NO: 235 - DNA, 10 bp, Artificial Sequence
  Mutant lox sites
acgtaatagt

SEQ ID NO: 236 - DNA, 10 bp, Artificial Sequence
  Mutant lox sites
taaatgtacg

SEQ ID NO: 237 - DNA, 10 bp, Artificial Sequence
  Mutant lox sites
tatgcaaatg

SEQ ID NO: 238 - DNA, 10 bp, Artificial Sequence
  Mutant lox sites
agtatagcta

SEQ ID NO: 239 - DNA, 10 bp, Artificial Sequence
  Mutant lox sites
gtaaatgcat

SEQ ID NO: 240 - DNA, 10 bp, Artificial Sequence
  Mutant lox sites
atgcataagt

SEQ ID NO: 241 - DNA, 10 bp, Artificial Sequence
  Mutant lox sites
atgtatgctt

SEQ ID NO: 242 - DNA, 10 bp, Artificial Sequence
  Mutant lox sites
ttcgtatgta

SEQ ID NO: 243 - DNA, 10 bp, Artificial Sequence
  Mutant lox sites
tgtatgcatt

SEQ ID NO: 244 - DNA, 10 bp, Artificial Sequence
  Mutant lox sites
gttattgcat

SEQ ID NO: 245 - DNA, 10 bp, Artificial Sequence
  Mutant lox sites
atgctattgt

SEQ ID NO: 246 - DNA, 10 bp, Artificial Sequence
  Mutant lox sites
cgttatgtta

SEQ ID NO: 247 - DNA, 10 bp, Artificial Sequence
  Mutant lox sites
tattgtatgc

SEQ ID NO: 248 - DNA, 10 bp, Artificial Sequence
  Mutant lox sites
atgcattttg

SEQ ID NO: 249 - DNA, 10 bp, Artificial Sequence
  Mutant lox sites
aacttgttcg

SEQ ID NO: 250 - DNA, 10 bp, Artificial Sequence
  Mutant lox sites
ggcaattttt

SEQ ID NO: 251 - DNA, 10 bp, Artificial Sequence
  Mutant lox sites
gcttataatg

SEQ ID NO: 252 - DNA, 10 bp, Artificial Sequence
  Mutant lox sites
agtgcttaat

SEQ ID NO: 253 - DNA, 10 bp, Artificial Sequence
  Mutant lox sites
atattatggc

SEQ ID NO: 254 - DNA, 10 bp, Artificial Sequence
  Mutant lox sites
ttatgtgaca

SEQ ID NO: 255 - DNA, 10 bp, Artificial Sequence
  Mutant lox sites
catgtgattt

SEQ ID NO: 256 - DNA, 10 bp, Artificial Sequence
  Mutant lox sites
tagtacttag

SEQ ID NO: 257 - DNA, 10 bp, Artificial Sequence
  Mutant lox sites
ggatctttaa

SEQ ID NO: 258 - DNA, 10 bp, Artificial Sequence
  Mutant lox sites
attgtgtatc

SEQ ID NO: 259 - DNA, 10 bp, Artificial Sequence
  Mutant lox sites
ttctaatagg

SEQ ID NO: 260 - DNA, 10 bp, Artificial Sequence
  Mutant lox sites
catgatgtta

SEQ ID NO: 261 - DNA, 10 bp, Artificial Sequence
  Mutant lox sites
taggcatgta

SEQ ID NO: 262 - DNA, 10 bp, Artificial Sequence
  Mutant lox sites
acttgtctag

SEQ ID NO: 263 - DNA, 10 bp, Artificial Sequence
  Mutant lox sites
cagtttgacg

SEQ ID NO: 264 - DNA, 10 bp, Artificial Sequence
  Mutant lox sites
cgtaggactt

SEQ ID NO: 265 - DNA, 10 bp, Artificial Sequence
  Mutant lox sites
aatgtctgag

SEQ ID NO: 266 - DNA, 10 bp, Artificial Sequence
  Mutant lox sites
tcaactgtgt

SEQ ID NO: 267 - DNA, 10 bp, Artificial Sequence
  Mutant lox sites
ggctcgttaa

SEQ ID NO: 268 - DNA, 10 bp, Artificial Sequence
  Mutant lox sites
catttaaggg

SEQ ID NO: 269 - DNA, 10 bp, Artificial Sequence
  Mutant lox sites
atcgggtatc

SEQ ID NO: 270 - DNA, 10 bp, Artificial Sequence
  Mutant lox sites
tggttaatcc

SEQ ID NO: 271 - DNA, 9 bp, Artificial Sequence
  Mutant lox sites
agagattct

SEQ ID NO: 272 - DNA, 9 bp, Artificial Sequence
  Mutant lox sites
tatatacgc

SEQ ID NO: 273 - DNA, 9 bp, Artificial Sequence
  Mutant lox sites
gaaattacg

SEQ ID NO: 274 - DNA, 9 bp, Artificial Sequence
  Mutant lox sites
atttccgaa

SEQ ID NO: 275 - DNA, 9 bp, Artificial Sequence
  Mutant lox sites
ccaattata

SEQ ID NO: 276 - DNA, 9 bp, Artificial Sequence
  Mutant lox sites
ttagggatt

SEQ ID NO: 277 - DNA, 9 bp, Artificial Sequence
  Mutant lox sites
attaaacgg

SEQ ID NO: 278 - DNA, 9 bp, Artificial Sequence
  Mutant lox sites
gcgtttatt

SEQ ID NO: 279 - DNA, 9 bp, Artificial Sequence
  Mutant lox sites
ttagcgaat

SEQ ID NO: 280 - DNA, 9 bp, Artificial Sequence
  Mutant lox sites
ctctttatc

SEQ ID NO: 281 - DNA, 9 bp, Artificial Sequence
  Mutant lox sites
agtgatata

SEQ ID NO: 282 - DNA, 9 bp, Artificial Sequence
  Mutant lox sites
tactcatat

SEQ ID NO: 283 - DNA, 9 bp, Artificial Sequence
  Mutant lox sites
caaattttg

SEQ ID NO: 284 - DNA, 9 bp, Artificial Sequence
  Mutant lox sites
gtttaaaac

SEQ ID NO: 285 - DNA, 9 bp, Artificial Sequence
  Mutant lox sites
tattgcatt

SEQ ID NO: 286 - DNA, 9 bp, Artificial Sequence
  Mutant lox sites
aaaccttaa

SEQ ID NO: 287 - DNA, 9 bp, Artificial Sequence
  Mutant lox sites
attatggta

SEQ ID NO: 288 - DNA, 9 bp, Artificial Sequence
  Mutant lox sites
ttgattact

SEQ ID NO: 289 - DNA, 9 bp, Artificial Sequence
  Mutant lox sites
acattatag

SEQ ID NO: 290 - DNA, 9 bp, Artificial Sequence
  Mutant lox sites
ttagcaata

SEQ ID NO: 291 - DNA, 9 bp, Artificial Sequence
  Mutant lox sites
aaatcttat

SEQ ID NO: 292 - DNA, 9 bp, Artificial Sequence
  Mutant lox sites
ttttttgtt

SEQ ID NO: 293 - DNA, 9 bp, Artificial Sequence
  Mutant lox sites
acaaaaaaa

SEQ ID NO: 294 - DNA, 9 bp, Artificial Sequence
  Mutant lox sites
ttattatga

SEQ ID NO: 295 - DNA, 9 bp, Artificial Sequence
  Mutant lox sites
aaacatttt

SEQ ID NO: 296 - DNA, 9 bp, Artificial Sequence
  Mutant lox sites
gtatatata

SEQ ID NO: 297 - DNA, 9 bp, Artificial Sequence
  Mutant lox sites
atatttaac

SEQ ID NO: 298 - DNA, 9 bp, Artificial Sequence
  Mutant lox sites
taattgaat

SEQ ID NO: 299 - DNA, 9 bp, Artificial Sequence
  Mutant lox sites
atcatatat

SEQ ID NO: 300 - DNA, 9 bp, Artificial Sequence
  Mutant lox sites
aaatataca

SEQ ID NO: 301 - DNA, 9 bp, Artificial Sequence
  Mutant lox sites
aaaattttt

SEQ ID NO: 302 - DNA, 9 bp, Artificial Sequence
  Mutant lox sites
ttttaaaaa

SEQ ID NO: 303 - DNA, 9 bp, Artificial Sequence
  Mutant lox sites
atatatata

SEQ ID NO: 304 - DNA, 9 bp, Artificial Sequence
  Mutant lox sites
tatatatat

SEQ ID NO: 305 - DNA, 9 bp, Artificial Sequence
  Mutant lox sites
attttaaat

SEQ ID NO: 306 - DNA, 9 bp, Artificial Sequence
  Mutant lox sites
aatttaaat

SEQ ID NO: 307 - DNA, 9 bp, Artificial Sequence
  Mutant lox sites
tttaattta

SEQ ID NO: 308 - DNA, 9 bp, Artificial Sequence
  Mutant lox sites
attatataa

SEQ ID NO: 309 - DNA, 9 bp, Artificial Sequence
  Mutant lox sites
tattattat

SEQ ID NO: 310 - DNA, 9 bp, Artificial Sequence
  Mutant lox sites
atttttaaa

SEQ ID NO: 311 - DNA, 9 bp, Artificial Sequence
  Mutant lox sites
aagtagctt

SEQ ID NO: 312 - DNA, 9 bp, Artificial Sequence
  Mutant lox sites
cgatatatg

SEQ ID NO: 313 - DNA, 9 bp, Artificial Sequence
  Mutant lox sites
ttcgttgaa

SEQ ID NO: 314 - DNA, 9 bp, Artificial Sequence
  Mutant lox sites
atatgaatc

SEQ ID NO: 315 - DNA, 9 bp, Artificial Sequence
  Mutant lox sites
ggatctata

SEQ ID NO: 316 - DNA, 9 bp, Artificial Sequence
  Mutant lox sites
cttaattag

SEQ ID NO: 317 - DNA, 9 bp, Artificial Sequence
  Mutant lox sites
ttgtcgaat

SEQ ID NO: 318 - DNA, 9 bp, Artificial Sequence
  Mutant lox sites
taaagcgat

SEQ ID NO: 319 - DNA, 9 bp, Artificial Sequence
  Mutant lox sites
aattggaac

SEQ ID NO: 320 - DNA, 9 bp, Artificial Sequence
  Mutant lox sites
tcagtaata

SEQ ID NO: 321 - DNA, 9 bp, Artificial Sequence
  Mutant lox sites
gaagcttat

SEQ ID NO: 322 - DNA, 9 bp, Artificial Sequence
  Mutant lox sites
tagctatga

SEQ ID NO: 323 - DNA, 9 bp, Artificial Sequence
  Mutant lox sites
cttaagtag

SEQ ID NO: 324 - DNA, 9 bp, Artificial Sequence
  Mutant lox sites
taagtgaca

SEQ ID NO: 325 - DNA, 9 bp, Artificial Sequence
  Mutant lox sites
aattaatac

SEQ ID NO: 326 - DNA, 9 bp, Artificial Sequence
  Mutant lox sites
gtgtcaatt

SEQ ID NO: 327 - DNA, 9 bp, Artificial Sequence
  Mutant lox sites
ttctatgga

SEQ ID NO: 328 - DNA, 9 bp, Artificial Sequence
  Mutant lox sites
aatatcgag

SEQ ID NO: 329 - DNA, 9 bp, Artificial Sequence
  Mutant lox sites
catatttag

SEQ ID NO: 330 - DNA, 9 bp, Artificial Sequence
  Mutant lox sites
ttgatacaa

SEQ ID NO: 331 - DNA, 9 bp, Artificial Sequence
  Mutant lox sites
acgttagta

SEQ ID NO: 332 - DNA, 9 bp, Artificial Sequence
  Mutant lox sites
taacgttgt

SEQ ID NO: 333 - DNA, 9 bp, Artificial Sequence
  Mutant lox sites
cattatgag

SEQ ID NO: 334 - DNA, 9 bp, Artificial Sequence
  Mutant lox sites
tttgtaaac

SEQ ID NO: 335 - DNA, 9 bp, Artificial Sequence
  Mutant lox sites
ggatcaatt

SEQ ID NO: 336 - DNA, 9 bp, Artificial Sequence
  Mutant lox sites
agatttatg

SEQ ID NO: 337 - DNA, 9 bp, Artificial Sequence
  Mutant lox sites
atttttagc

SEQ ID NO: 338 - DNA, 9 bp, Artificial Sequence
  Mutant lox sites
ttaaaggat

SEQ ID NO: 339 - DNA, 9 bp, Artificial Sequence
  Mutant lox sites
caaaattgt

SEQ ID NO: 340 - DNA, 9 bp, Artificial Sequence
  Mutant lox sites
tcttggtaa

SEQ ID NO: 341 - DNA, 9 bp, Artificial Sequence
  Mutant lox sites
cgatttgaa

SEQ ID NO: 342 - DNA, 9 bp, Artificial Sequence
  Mutant lox sites
aatcgtttg

SEQ ID NO: 343 - DNA, 9 bp, Artificial Sequence
  Mutant lox sites
tctatgtgt

SEQ ID NO: 344 - DNA, 9 bp, Artificial Sequence
  Mutant lox sites
ggttaaatc

SEQ ID NO: 345 - DNA, 9 bp, Artificial Sequence
  Mutant lox sites
aactgtgta

SEQ ID NO: 346 - DNA, 9 bp, Artificial Sequence
  Mutant lox sites
tttgtacag

SEQ ID NO: 347 - DNA, 9 bp, Artificial Sequence
  Mutant lox sites
cggaaattt

SEQ ID NO: 348 - DNA, 9 bp, Artificial Sequence
  Mutant lox sites
atcttggat

SEQ ID NO: 349 - DNA, 9 bp, Artificial Sequence
  Mutant lox sites
tattcggaa

SEQ ID NO: 350 - DNA, 9 bp, Artificial Sequence
  Mutant lox sites
aagtgactt

SEQ ID NO: 351 - DNA, 9 bp, Artificial Sequence
  Mutant lox sites
catgattag

SEQ ID NO: 352 - DNA, 9 bp, Artificial Sequence
  Mutant lox sites
gcgtttaaa

SEQ ID NO: 353 - DNA, 9 bp, Artificial Sequence
  Mutant lox sites
aaatcggtt

SEQ ID NO: 354 - DNA, 9 bp, Artificial Sequence
  Mutant lox sites
taagtatgc

SEQ ID NO: 355 - DNA, 9 bp, Artificial Sequence
  Mutant lox sites
tttcagaga

SEQ ID NO: 356 - DNA, 9 bp, Artificial Sequence
  Mutant lox sites
agctgaatt

SEQ ID NO: 357 - DNA, 9 bp, Artificial Sequence
  Mutant lox sites
cttaatgga

SEQ ID NO: 358 - DNA, 9 bp, Artificial Sequence
  Mutant lox sites
ggtaaatct

SEQ ID NO: 359 - DNA, 9 bp, Artificial Sequence
  Mutant lox sites
acgtattag

SEQ ID NO: 360 - DNA, 9 bp, Artificial Sequence
  Mutant lox sites
agatttagc

SEQ ID NO: 361 - DNA, 9 bp, Artificial Sequence
  Mutant lox sites
tatctgtag

SEQ ID NO: 362 - DNA, 9 bp, Artificial Sequence
  Mutant lox sites
ctggatatt

SEQ ID NO: 363 - DNA, 9 bp, Artificial Sequence
  Mutant lox sites
tgtattcga

SEQ ID NO: 364 - DNA, 9 bp, Artificial Sequence
  Mutant lox sites
atatgcttg

SEQ ID NO: 365 - DNA, 9 bp, Artificial Sequence
  Mutant lox sites
gattttgac

SEQ ID NO: 366 - DNA, 9 bp, Artificial Sequence
  Mutant lox sites
aagtcgttt

SEQ ID NO: 367 - DNA, 9 bp, Artificial Sequence
  Mutant lox sites
gtactttga

SEQ ID NO: 368 - DNA, 9 bp, Artificial Sequence
  Mutant lox sites
tcattgtga

SEQ ID NO: 369 - DNA, 9 bp, Artificial Sequence
  Mutant lox sites
attagcgtt

SEQ ID NO: 370 - DNA, 9 bp, Artificial Sequence
  Mutant lox sites
tgtgttcaa

SEQ ID NO: 371 - DNA, 9 bp, Artificial Sequence
  Mutant lox sites
ttggacagt

SEQ ID NO: 372 - DNA, 9 bp, Artificial Sequence
  Mutant lox sites
gatttggac

SEQ ID NO: 373 - DNA, 9 bp, Artificial Sequence
  Mutant lox sites
agcatgttg

SEQ ID NO: 374 - DNA, 9 bp, Artificial Sequence
  Mutant lox sites
ctgggtata

SEQ ID NO: 375 - DNA, 9 bp, Artificial Sequence
  Mutant lox sites
aattgtcgg

SEQ ID NO: 376 - DNA, 9 bp, Artificial Sequence
  Mutant lox sites
gacatgttg

SEQ ID NO: 377 - DNA, 9 bp, Artificial Sequence
  Mutant lox sites
ggtttcgaa

SEQ ID NO: 378 - DNA, 9 bp, Artificial Sequence
  Mutant lox sites
aaggtttgc

SEQ ID NO: 379 - DNA, 9 bp, Artificial Sequence
  Mutant lox sites
ctgtaagtg

SEQ ID NO: 380 - DNA, 9 bp, Artificial Sequence
  Mutant lox sites
tgtagcgat

SEQ ID NO: 381 - DNA, 9 bp, Artificial Sequence
  Mutant lox sites
ctgattagc

SEQ ID NO: 382 - DNA, 9 bp, Artificial Sequence
  Mutant lox sites
tcatggtca

SEQ ID NO: 383 - DNA, 9 bp, Artificial Sequence
  Mutant lox sites
ggcatactt

SEQ ID NO: 384 - DNA, 9 bp, Artificial Sequence
  Mutant lox sites
attcactgg

SEQ ID NO: 385 - DNA, 9 bp, Artificial Sequence
  Mutant lox sites
tgcgcatta

SEQ ID NO: 386 - DNA, 9 bp, Artificial Sequence
  Mutant lox sites
aggctctat

SEQ ID NO: 387 - DNA, 9 bp, Artificial Sequence
  Mutant lox sites
gtcttacag

SEQ ID NO: 388 - DNA, 9 bp, Artificial Sequence
  Mutant lox sites
acttggtca

SEQ ID NO: 389 - DNA, 9 bp, Artificial Sequence
  Mutant lox sites
cggatttac

SEQ ID NO: 390 - DNA, 9 bp, Artificial Sequence
  Mutant lox sites
gtcatcgta